SEQUENCE LISTING (37 sequences)

SEQ ID NO: 1 - protein, 201 aa, Homo sapiens
MALSWVLTVLSLLPLLEAQIPLCANLVPVPITNATLDQITGKWFYIASAFRNEEYNKSVQEIQATFFYFTPNKTEDTIFLREYQTRQDQCIYNTTYLNVQRENGTISRYVGGQEHFAHLLILRDTKTYMLAFDVNDEKNWGLSVYADKPETTKEQLGEFYEALDCLRIPKSDVVYTDWKKDKCEPLEKQHEKERKQEEGES

SEQ ID NO: 2 - protein, 418 aa, Homo sapiens
MPSSVSWGILLLAGLCCLVPVSLAEDPQGDAAQKTDTSHHDQDHPTFNKITPNLAEFAFSLYRQLAHQSNSTNIFFSPVSIATAFAMLSLGTKADTHDEILEGLNFNLTEIPEAQIHEGFQELLRTLNQPDSQLQLTTGNGLFLSEGLKLVDKFLEDVKKLYHSEAFTVNFGDTEEAKKQINDYVEKGTQGKIVDLVKELDRDTVFALVNYIFFKGKWERPFEVKDTEEEDFHVDQVTTVKVPMMKRLGMFNIQHCKKLSSWVLLMKYLGNATAIFFLPDEGKLQHLENELTHDIITKFLENEDRRSASLHLPKLSITGTYDLKSVLGQLGITKVFSNGADLSGVTEEAPLKLSKAVHKAVLTIDEKGTEAAGAMFLEAIPMSIPPEVKFNKPFVFLMIEQNTKSPLFMGKVVNPTQK

SEQ ID NO: 3 - protein, 423 aa, Homo sapiens
MERMLPLLALGLLAAGFCPAVLCHPNSPLDEENLTQENQDRGTHVDLGLASANVDFAFSLYKQLVLKAPDKNVIFSPLSISTALAFLSLGAHNTTLTEILKGLKFNLTETSEAEIHQSFQHLLRTLNQSSDELQLSMGNAMFVKEQLSLLDRFTEDAKRLYGSEAFATDFQDSAAAKKLINDYVKNGTRGKITDLIKDLDSQTMMVLVNYIFFKAKWEMPFDPQDTHQSRFYLSKKKWVMVPMMSLHHLTIPYFRDEELSCTVVELKYTGNASALFILPDQDKMEEVEAMLLPETLKRWRDSLEFREIGELYLPKFSISRDYNLNDILLQLGIEEAFTSKADLSGITGARNLAVSQVVHKAVLDVFEEGTEASAATAVKITLLSALVETRTIVRFNRPFLMIIVPTDTQNIFFMSKVTNPKQA

SEQ ID NO: 4 - protein, 511 aa, Homo sapiens
MKFFLLLFTIGFCWAQYSPNTQQGRTSIVHLFEWRWVDIALECERYLAPKGFGGVQVSPPNENVAIHNPFRPWWERYQPVSYKLCTRSGNEDEFRNMVTRCNNVGVRIYVDAVINHMSGNAVSAGTSSTCGSYFNPGSRDFPAVPYSGWDFNDGKCKTGSGDIENYNDATQVRDCRLVGLLDLALEKDYVRSKIAEYMNHLIDIGVAGFRLDASKHMWPGDIKAILDKLHNLNSNWFPAGSKPFIYQEVIDLGGEPIKSSDYFGNGRVTEFKYGAKLGTVIRKWNGEKMSYLKNWGEGWGFMPSDRALVFVDNHDNQRGHGAGGASILTFWDARLYKMAVGFMLAHPYGFTRVMSSYRWPRQFQNGNDVNDWVGPPNNNGVIKEVTINPDTTCGNDWVCEHRWRQIRNMVNFRNVVDGQPFTNWYDNGSNQVAFGRGNRGFIVFNNDDWTFSLTLQTGLPAGTYCDVISGDKINGNCTGIKIYVSDDGKAHFSISNSAEDPFIAIHAESKL

SEQ ID NO: 5 - protein, 346 aa, Homo sapiens
MAMVSEFLKQAWFIENEEQEYVQTVKSSKGGPGSAVSPYPTFNPSSDVAALHKAIMVKGVDEATIIDILTKRNNAQRQQIKAAYLQETGKPLDETLKKALTGHLEEVVLALLKTPAQFDADELRAAMKGLGTDEDTLIEILASRTNKEIRDINRVYREELKRDLAKDITSDTSGDFRNALLSLAKGDRSEDFGVNEDLADSDARALYEAGERRKGTDVNVFNTILTTRSYPQLRRVFQKYTKYSKHDMNKVLDLELKGDIEKCLTAIVKCATSKPAFFAEKLHQAMKGVGTRHKALIRIMVSRSEIDMNDIKAFYQKMYGISLCQAILDETKGDYEKILVALCGGN

SEQ ID NO: 6 - protein, 267 aa, Homo sapiens
MKAAVLTLAVLFLTGSQARHFWQQDEPPQSPWDRVKDLATVYVDVLKDSGRDYVSQFEGSALGKQLNLKLLDNWDSVTSTFSKLREQLGPVTQEFWDNLEKETEGLRQEMSKDLEEVKAKVQPYLDDFQKKWQEEMELYRQKVEPLRAELQEGARQKLHELQEKLSPLGEEMRDRARAHVDALRTHLAPYSDELRQRLAARLEALKENGGARLAEYHAKATEHLSTLSEKAKPALEDLRQGLLPVLESFKVSFLSALEEYTKKLNTQ

SEQ ID NO: 7 - protein, 261 aa, Homo sapiens
MASPDWGYDDKNGPEQWSKLYPIANGNNQSPVDIKTSETKHDTSLKPISVSYNPATAKEIINVGHSFHVNFEDNDNRSVLKGGPFSDSYRLFQFHFHWGSTNEHGSEHTVDGVKYSAELHVAHWNSAKYSSLAEAASKADGLAVIGVLMKVGEANPKLQKVLDALQAIKTKGKRAPFTNFDPSTLLPSSLDFWTYPGSLTHPPLYESVTWIICKESISVSSEQLAQFRSLLSNVEGDNAVPMQHNNRPTQPLKGRTVRASF

SEQ ID NO: 8 - protein, 412 aa, Homo sapiens
MQPSSLLPLALCLLAAPASALVRIPLHKFTSIRRTMSEVGGSVEDLIAKGPVSKYSQAVPAVTEGPIPEVLKNYMDAQYYGEIGIGTPPQCFTVVFDTGSSNLWVPSIHCKLLDIACWIHHKYNSDKSSTYVKNGTSFDIHYGSGSLSGYLSQDTVSVPCQSASSASALGGVKVERQVFGEATKQPGITFIAAKFDGILGMAYPRISVNNVLPVFDNLMQQKLVDQNIFSFYLSRDPDAQPGGELMLGGTDSKYYKGSLSYLNVTRKAYWQVHLDQVEVASGLTLCKEGCEAIVDTGTSLMVGPVDEVRELQKAIGAVPLIQGEYMIPCEKVSTLPAITLKLGGKGYKLSPEDYTLKVSQAGKTLCLSGFMGMDIPPPSGPLWILGDVFIGRYYTVFDRDNNRVGFAEAARL

SEQ ID NO: 9 - protein, 331 aa, Homo sapiens
MKRLVCVLLVCSSAVAQLHKDPTLDHHWHLWKKTYGKQYKEKNEEAVRRLIWEKNLKFVMLHNLEHSMGMHSYDLGMNHLGDMTSEEVMSLMSSLRVPSQWQRNITYKSNPNRILPDSVDWREKGCVTEVKYQGSCGACWAFSAVGALEAQLKLKTGKLVSLSAQNLVDCSTEKYGNKGCNGGFMTTAFQYIIDNKGIDSDASYPYKAMDQKCQYDSKYRAATCSKYTELPYGREDVLKEAVANKGPVSVGVDARHPSFFLYRSGVYYEPSCTQNVNHGVLVVGYGDLNGKEYWLVKNSWGHNFGEEGYIRMARNKGNHCGIASFPSYPEI

SEQ ID NO: 10 - protein, 252 aa, Homo sapiens
MGPPSASPHRECIPWQGLLLTASLLNFWNPPTTAKLTIESMPLSVAEGKEVLLLVHNLPQHLFGYSWYKGERVDGNSLIVGYVIGTQQATPGAAYSGRETIYTNASLLIQNVTQNDIGFYTLQVIKSDLVNEEATGQFHVYQENAPGLPVGAVAGIVTGVLVGVALVAALVCFLLLAKTGRTSIQRDLKEQQPQALAPGRGPSHSSAFSMSPLSTAQAPLPNPRTAASIYEELLKHDTNIYCRMDHKAEVAS

SEQ ID NO: 11 - protein, 449 aa, Homo sapiens
MMKTLLLFVGLLLTWESGQVLGDQTVSDNELQEMSNQGSKYVNKEIQNAVNGVKQIKTLIEKTNEERKTLLSNLEEAKKKKEDALNETRESETKLKELPGVCNETMMALWEECKPCLKQTCMKFYARVCRSGSGLVGRQLEEFLNQSSPFYFWMNGDRIDSLLENDRQQTHMLDVMQDHFSRASSIIDELFQDRFFTREPQDTYHYLPFSLPHRRPHFFFPKSRIVRSLMPFSPYEPLNFHAMFQPFLEMIHEAQQAMDIHFHSPAFQHPPTEFIREGDDDRTVCREIRHNSTGCLRMKDQCDKCREILSVDCSTNNPSQAKLRRELDESLQVAERLTRKYNELLKSYQWKMLNTSSLLEQLNEQFNWVSRLANLTQGEDQYYLRVTTVASHTSDSDVPSGVTEVVVKLFDSDPITVTVPVEVSRKNPKFMETVAEKALQEYRKKHREE

SEQ ID NO: 12 - protein, 781 aa, Homo sapiens
MATQADLMELDMAMEPDRKAAVSHWQQQSYLDSGIHSGATTTAPSLSGKGNPEEEDVDTSQVLYEWEQGFSQSFTQEQVADIDGQYAMTRAQRVRAAMFPETLDEGMQIPSTQFDAAHPTNVQRLAEPSQMLKHAVVNLINYQDDAELATRAIPELTKLLNDEDQVVVNKAAVMVHQLSKKEASRHAIMRSPQMVSAIVRTMQNTNDVETARCTAGTLHNLSHHREGLLAIFKSGGIPALVKMLGSPVDSVLFYAITTLHNLLLHQEGAKMAVRLAGGLQKMVALLNKTNVKFLAITTDCLQILAYGNQESKLIILASGGPQALVNIMRTYTYEKLLWTTSRVLKVLSVCSSNKPAIVEAGGMQALGLHLTDPSQRLVQNCLWTLRNLSDAATKQEGMEGLLGTLVQLLGSDDINVVTCAAGILSNLTCNNYKNKMMVCQVGGIEALVRTVLRAGDREDITEPAICALRHLTSRHQEAEMAQNAVRLHYGLPVVVKLLHPPSHWPLIKATVGLIRNLALCPANHAPLREQGAIPRLVQLLVRAHQDTQRRTSMGGTQQQFVEGVRMEEIVEGCTGALHILARDVHNRIVIRGLNTIPLFVQLLYSPIENIQRVAAGVLCELAQDKEAAEAIEAEGATAPLTELLHSRNEGVATYAAAVLFRMSEDKPQDYKKRLSVELTSSLFRTEPMAWNETADLGLDIGAQGEPLGYRQDDPSYRSFHSGGYGQDALGMDPMMEHEMGGHHPGADYPVDGLPDLGHAQDLMDGLPPGDSNQLAWFDTDL

SEQ ID NO: 13 - protein, 1663 aa, Homo sapiens
MGPTSGPSLLLLLLTHLPLALGSPMYSIITPNILRLESEETMVLEAHDAQGDVPVTVTVHDFPGKKLVLSSEKTVLTPATNHMGNVTFTIPANREFKSEKGRNKFVTVQATFGTQVVEKVVLVSLQSGYLFIQTDKTIYTPGSTVLYRIFTVNHKLLPVGRTVMVNIENPEGIPVKQDSLSSQNQLGVLPLSWDIPELVNMGQWKIRAYYENSPQQVFSTEFEVKEYVLPSFEVIVEPTEKFYYIYNEKGLEVTITARFLYGKKVEGTAFVIFGIQDGEQRISLPESLKRIPIEDGSGEVVLSRKVLLDGVQNPRAEDLVGKSLYVSATVILHSGSDMVQAERSGIPIVTSPYQIHFTKTPKYFKPGMPFDLMVFVTNPDGSPAYRVPVAVQGEDTVQSLTQGDGVAKLSINTHPSQKPLSITVRTKKQELSEAEQATRTMQALPYSTVGNSNNYLHLSVLRTELRPGETLNVNFLLRMDRAHEAKIRYYTYLIMNKGRLLKAGRQVREPGQDLVVLPLSITTDFIPSFRLVAYYTLIGASGQREVVADSVWVDVKDSCVGSLVVKSGQSEDRQPVPGQQMTLKIEGDHGARVVLVAVDKGVFVLNKKNKLTQSKIWDVVEKADIGCTPGSGKDYAGVFSDAGLTFTSSSGQQTAQRAELQCPQPAARRRRSVQLTEKRMDKVGKYPKELRKCCEDGMRENPMRFSCQRRTRFISLGEACKKVFLDCCNYITELRRQHARASHLGLARSNLDEDIIAEENIVSRSEFPESWLWNVEDLKEPPKNGISTKLMNIFLKDSITTWEILAVSMSDKKGICVADPFEVTVMQDFFIDLRLPYSVVRNEQVEIRAVLYNYRQNQELKVRVELLHNPAFCSLATTKRRHQQTVTIPPKSSLSVPYVIVPLKTGLQEVEVKAAVYHHFISDGVRKSLKVVPEGIRMNKTVAVRTLDPERLGREGVQKEDIPPADLSDQVPDTESETRILLQGTPVAQMTEDAVDAERLKHLIVTPSGCGEQNMIGMTPTVIAVHYLDETEQWEKFGLEKRQGALELIKKGYTQQLAFRQPSSAFAAFVKRAPSTWLTAYVVKVFSLAVNLIAIDSQVLCGAVKWLILEKQKPDGVFQEDAPVIHQEMIGGLRNNNEKDMALTAFVLISLQEAKDICEEQVNSLPGSITKAGDFLEANYMNLQRSYTVAIAGYALAQMGRLKGPLLNKFLTTAKDKNRWEDPGKQLYNVEATSYALLALLQLKDFDFVPPVVRWLNEQRYYGGGYGSTQATFMVFQALAQYQKDAPDHQELNLDVSLQLPSRSSKITHRIHWESASLLRSEETKENEGFTVTAEGKGQGTLSVVTMYHAKAKDQLTCNKFDLKVTIKPAPETEKRPQDAKNTMILEICTRYRGDQDATMSILDISMMTGFAPDTDDLKQLANGVDRYISKYELDKAFSDRNTLIIYLDKVSHSEDDCLAFKVHQYFNVELIQPGAVKVYAYYNLEESCTRFYHPEKEDGKLNKLCRDELCRCAEENCFIQKSDDKVTLEERLDKACEPGVDYVYKTRLVKVQLSNDFDEYIMAIEQTIKSGSDEVQVGQQRTFISPIKCREALKLEEKKHYLMWGLSSDFWGEKPNLSYIIGKDTWVEHWPEEDECQDEENQKQCQDLGAFTESMVVFGCPN

SEQ ID NO: 14 - protein, 559 aa, Homo sapiens
MSACRSFAVAICILEISILTAQYTTSYDPELTESSGSASHIDCRMSPWSEWSQCDPCLRQMFRSRSIEVFGQFNGKRCTDAVGDRRQCVPTEPCEDAEDDCGNDFQCSTGRCIKMRLRCNGDNDCGDFSDEDDCESEPRPPCRDRVVEESELARTAGYGINILGMDPLSTPFDNEFYNGLCNRDRDGNTLTYYRRPWNVASLIYETKGEKNFRTEHYEEQIEAFKSIIQEKTSNFNAAISLKFTPTETNKAEQCCEETASSISLHGKGSFRFSYSKNETYQLFLSYSSKKEKMFLHVKGEIHLGRFVMRNRDVVLTTTFVDDIKALPTTYEKGEYFAFLETYGTHYSSSGSLGGLYELIYVLDKASMKRKGVELKDIKRCLGYHLDVSLAFSEISVGAEFNKDDCVKRGEGRAVNITSENLIDDVVSLIRGGTRKYAFELKEKLLRGTVIDVTDFVNWASSINDAPVLISQKLSPIYNLVPVKMKNAHLKKQNLERAIEDYINEFSVRKCHTCQNGGTVILMDGKCLCACPFKFEGIACEISKQKISEGLPALEFPNEK

SEQ ID NO: 15 - protein, 224 aa, Homo sapiens
MEKLLCFLVLTSLSHAFGQTDMSRKAFVFPKESDTSYVSLKAPLTKPLKAFTVCLHFYTELSSTRGYSIFSYATKRQDNEILIFWSKDIGYSFTVGGSEILFEVPEVTVAPVHICTSWESASGIVEFWVDGKPRVRKSLKKGYTVGAEASIILGQEQDSFGGNFEGSQSLVGDIGNVNMWDFVLSPDEINTIYLGGPFSPNVLNWRALKYEVQGEVFTKPQLWP

SEQ ID NO: 16 - protein, 554 aa, Homo sapiens
MTAPGAAGRCPPTTWLGSLLLLVCLLASRSITEEVSEYCSHMIGSGHLQSLQRLIDSQMETSCQITFEFVDQEQLKDPVCYLKKAFLLVQDIMEDTMRFRDNTPNAIAIVQLQELSLRLKSCFTKDYEEHDKACVRTFYETPLQLLEKVKNVFNETKNLLDKDWNIFSKNCNNSFAECSSQDVVTKPDCNCLYPKAIPSSDPASVSPHQPLAPSMAPVAGLTWEDSEGTEGSSLLPGEQPLHTVDPGSAKQRPPRSTCQSFEPPETPVVKDSTIGGSPQPRPSVGAFNPGMEDILDSAMGTNWVPEEASGEASEIPVPQGTELSPSRPGGGSMQTEPARPSNFLSASSPLPASAKGQQPADVTGTALPRVGPVRPTGQDWNHTPQKTDHPSALLRDPPEPGSPRISSLRPQGLSNPSTLSAQPQLSRSHSSGSVLPLGELEGRRSTRDRRSPAEPEGGPASEGAARPLPRFNSVPLTDTGHERQSEGSFSPQLQESVFHLLVPSVILVLLAVGGLLFYRWRRRSHQEPQRADSPLEQPEGSPLTQDDRQVELPV

SEQ ID NO: 17 - protein, 766 aa, Homo sapiens
MKTPWKVLLGLLGAAALVTIITVPVVLLNKGTDDATADSRKTYTLTDYLKNTYRLKLYSLRWISDHEYLYKQENNILVFNAEYGNSSVFLENSTFDEFGHSINDYSISPDGQFILLEYNYVKQWRHSYTASYDIYDLNKRQLITEERIPNNTQWVTWSPVGHKLAYVWNNDIYVKIEPNLPSYRITWTGKEDIIYNGITDWVYEEEVFSAYSALWWSPNGTFLAYAQFNDTEVPLIEYSFYSDESLQYPKTVRVPYPKAGAVNPTVKFFVVNTDSLSSVTNATSIQITAPASMLIGDHYLCDVTWATQERISLQWLRRIQNYSVMDICDYDESSGRWNCLVARQHIEMSTTGWVGRFRPSEPHFTLDGNSFYKIISNEEGYRHICYFQIDKKDCTFITKGTWEVIGIEALTSDYLYYISNEYKGMPGGRNLYKIQLSDYTKVTCLSCELNPERCQYYSVSFSKEAKYYQLRCSGPGLPLYTLHSSVNDKGLRVLEDNSALDKMLQNVQMPSKKLDFIILNETKFWYQMILPPHFDKSKKYPLLLDVYAGPCSQKADTVFRLNWATYLASTENIIVASFDGRGSGYQGDKIMHAINRRLGTFEVEDQIEAARQFSKMGFVDNKRIAIWGWSYGGYVTSMVLGSGSGVFKCGIAVAPVSRWEYYDSVYTERYMGLPTPEDNLDHYRNSTVMSRAENFKQVEYLLIHGTADDNVHFQQSAQISKALVDVGVDFQAMWYTDEDHGIASSTAHQHIYTHMSHFIKQCFSLP

SEQ ID NO: 18 - protein, 328 aa, Homo sapiens
MAAGIVASRRLRDLLTRRLTGSNYPGLSISLRLTGSSAQEEASGVALGEAPDHSYESLRVTSAQKHVLHVQLNRPNKRNAMNKVFWREMVECFNKISRDADCRAVVISGAGKMFTAGIDLMDMASDILQPKGDDVARISWYLRDIITRYQETFNVIERCPKPVIAAVHGGCIGGGVDLVTACDIRYCAQDAFFQVKEVDVGLAADVGTLQRLPKVIGNQSLVNELAFTARKMMADEALGSGLVSRVFPDKEVMLDAALALAAEISSKSPVAVQSTKVNLLYSRDHSVAESLNYVASWNMSMLQTQDLVKSVQATTENKELKTVTFSKL

SEQ ID NO: 19 - protein, 323 aa, Homo sapiens
MAEKFDCHYCRDPLQGKKYVQKDGHHCCLKCFDKFCANTCVECRKPIGADSKEVHYKNRFWHDTCFRCAKCLHPLANETFVAKDNKILCNKCTTREDSPKCKGCFKAIVAGDQNVEYKGTVWHKDCFTCSNCKQVIGTGSFFPKGEDFYCVTCHETKFAKHCVKCNKAITSGGITYQDQPWHADCFVCVTCSKKLAGQRFTAVEDQYYCVDCYKNFVAKKCAGCKNPITGKRTVSRVSHPVSKARKPPVCHGKRLPLTLFPSANLRGRHPGGERTCPSWVVVLYRKNRSLAAPRGPGLVKAPVWWPMKDNPGTTTASTAKNAP

SEQ ID NO: 20 - protein, 491 aa, Homo sapiens
MKRMVSWSFHKLKTMKHLLLLLLCVFLVKSQGVNDNEEGFFSARGHRPLDKKREEAPSLRPAPPPISGGGYRARPAKAAATQKKVERKAPDAGGCLHADPDLGVLCPTGCQLQEALLQQERPIRNSVDELNNNVEAVSQTSSSSFQYMYLLKDLWQKRQKQVKDNENVVNEYSSELEKHQLYIDETVNSNIPTNLRVLRSILENLRSKIQKLESDVSAQMEYCRTPCTVSCNIPVVSGKECEEIIRKGGETSEMYLIQPDSSVKPYRVYCDMNTENGGWTVIQNRQDGSVDFGRKWDPYKQGFGNVATNTDGKNYCGLPGEYWLGNDKISQLTRMGPTELLIEMEDWKGDKVKAHYGGFTVQNEANKYQISVNKYRGTAGNALMDGASQLMGENRTMTIHNGMFFSTYDRDNDGWLTSDPRKQCSKEDGGGWWYNRCHAANPNGRYYWGGQYTWDMAKHGTDDGVVWMNWKGSWYSMRKMSMKIRPFFPQQ

SEQ ID NO: 21 - protein, 453 aa, Homo sapiens
MSWSLHPRNLILYFYALLFLSSTCVAYVATRDNCCILDERFGSYCPTTCGIADFLSTYQTKVDKDLQSLEDILHQVENKTSEVKQLIKAIQLTYNPDESSKPNMIDAATLKSRKMLEEIMKYEASILTHDSSIRYLQEIYNSNNQKIVNLKEKVAQLEAQCQEPCKDTVQIHDITGKDCQDIANKGAKQSGLYFIKPLKANQQFLVYCEIDGSGNGWTVFQKRLDGSVDFKKNWIQYKEGFGHLSPTGTTEFWLGNEKIHLISTQSAIPYALRVELEDWNGRTSTADYAMFKVGPEADKYRLTYAYFAGGDAGDAFDGFDFGDDPSDKFFTSHNGMQFSTWDNDNDKFEGNCAEQDGSGWWMNKCHAGHLNGVYYQGGTYSKASTPNGYDNGIIWATWKTRWYSMKKTTMKIIPFNRLTIGEGQQHHLGGAKQVRPEHPAETEYDSLYPEDDL

SEQ ID NO: 22 - protein, 175 aa, Homo sapiens
MSSQIRQNYSTDVEAAVNSLVNLYLQASYTYLSLGFYFDRDDVALEGVSHFFRELAEEKREGYERLLKMQNQRGGRALFQDIKKPAEDEWGKTPDAMKAAMALEKKLNQALLDLHALGSARTDPHLCDFLETHFLDEEVKLIKKMGDHLTNLHRLGGPEAGLGEYLFERLTLKHD

SEQ ID NO: 23 - protein, 466 aa, Homo sapiens
MRAPGMRSRPAGPALLLLLLFLGAAESVRRAQPPRRYTPDWPSLDSRPLPAWFDEAKFGVFIHWGVFSVPAWGSEWFWWHWQGEGRPQYQRFMRDNYPPGFSYADFGPQFTARFFHPEEWADLFQAAGAKYVVLTTKHHEGFTNWPSPVSWNWNSKDVGPHRDLVGELGTALRKRNIRYGLYHSLLEWFHPLYLLDKKNGFKTQHFVSAKTMPELYDLVNSYKPDLIWSDGEWECPDTYWNSTNFLSWLYNDSPVKDEVVVNDRWGQNCSCHHGGYYNCEDKFKPQSLPDHKWEMCTSIDKFSWGYRRDMALSDVTEESEIISELVQTVSLGGNYLLNIGPTKDGLIVPIFQERLLAVGKWLSINGEAIYASKPWRVQWEKNTTSVWYTSKGSAVYAIFLHWPENGVLNLESPITTSTTKITMLGIQGDLKWSTDPDKGLFISLPQLPPSAVPAEFAWTIKLTGVK

SEQ ID NO: 24 - protein, 782 aa, Homo sapiens
MAPHRPAPALLCALSLALCALSLPVRAATASRGASQAGAPQGRVPEARPNSMVVEHPEFLKAGKEPGLQIWRVEKFDLVPVPTNLYGDFFTGDAYVILKTVQLRNGNLQYDLHYWLGNECSQDESGAAAIFTVQLDDYLNGRAVQHREVQGFESATFLGYFKSGLKYKKGGVASGFKHVVPNEVVVQRLFQVKGRRVVRATEVPVSWESFNNGDCFILDLGNNIHQWCGSNSNRYERLKATQVSKGIRDNERSGRARVHVSEEGTEPEAMLQVLGPKPALPAGTEDTAKEDAANRKLAKLYKVSNGAGTMSVSLVADENPFAQGALKSEDCFILDHGKDGKIFVWKGKQANTEERKAALKTASDFITKMDYPKQTQVSVLPEGGETPLFKQFFKNWRDPDQTDGLGLSYLSSHIANVERVPFDAATLHTSTAMAAQHGMDDDGTGQKQIWRIEGSNKVPVDPATYGQFYGGDSYIILYNYRHGGRQGQIIYNWQGAQSTQDEVAASAILTAQLDEELGGTPVQSRVVQGKEPAHLMSLFGGKPMIIYKGGTSREGGQTAPASTRLFQVRANSAGATRAVEVLPKAGALNSNDAFVLKTPSAAYLWVGTGASEAEKTGAQELLRVLRAQPVQVAEGSEPDGFWEALGGKAAYRTSPRLKDKKMDAHPPRLFACSNKIGRFVIEEVPGELMQEDLATDDVMLLDTWDQVFVWVGKDSQEEEKTEALTSAKRYIETDPANRDRRTPITVVKQGFEPPSFVGWFLGWDDDYWSVDPLDRAMAELAA

SEQ ID NO: 25 - protein, 406 aa, Homo sapiens
MSALGAVIALLLWGQLFAVDSGNDVTDIADDGCPKPPEIAHGYVEHSVRYQCKNYYKLRTEGDGVYTLNDKKQWINKAVGDKLPECEADDGCPKPPEIAHGYVEHSVRYQCKNYYKLRTEGDGVYTLNNEKQWINKAVGDKLPECEAVCGKPKNPANPVQRILGGHLDAKGSFPWQAKMVSHHNLTTGATLINEQWLLTTAKNLFLNHSENATAKDIAPTLTLYVGKKQLVEIEKVVLHPNYSQVDIGLIKLKQKVSVNERVMPICLPSKDYAEVGRVGYVSGWGRNANFKFTDHLKYVMLPVADQDQCIRHYEGSTVPEKKTPKSPVGVQPILNEHTFCAGMSKYQEDTCYGDAGSAFAVHDLEEDTWYATGILSFDKSCAVAEYGVYVKVTSIQDWVQKTIAEN

SEQ ID NO: 26 - protein, 314 aa, Homo sapiens
MRIAVICFCLLGITCAIPVKQADSGSSEEKQLYNKYPDAVATWLNPDPSQKQNLLAPQNAVSSEETNDFKQETLPSKSNESHDHMDDMDDEDDDDHVDSQDSIDSNDSDDVDDTDDSHQSDESHHSDESDELVTDFPTDLPATEVFTPVVPTVDTYDGRGDSVVYGLRSKSKKFRRPDIQYPDATDEDITSHMESEELNGAYKAIPVAQDLNAPSDWDSRGKDSYETSQLDDQSAETHSHKQSRLYKRKANDESNEHSDVIDSQELSKVSREFHSHEFHSHEDMLVVDPKSKEEDKHLKFRISHELDSASSEVN

SEQ ID NO: 27 - protein, 199 aa, Homo sapiens
MSSGNAKIGHPAPNFKATAVMPDGQFKDISLSDYKGKYVVFFFYPLDFTFVCPTEIIAFSDRAEEFKKLNCQVIGASVDSHFCHLAWVNTPKKQGGLGPMNIPLVSDPKRTIAQDYGVLKADEGISFRGLFIIDDKGILRQITVNDLPVGRSVDETLRLVQAFQFTDKHGEVCPAGWKPGSDTIKPDVQKSKEYFSKQK

SEQ ID NO: 28 - protein, 122 aa, Homo sapiens
MKLLTGLVFCSLVLGVSSRSFFSFLGEAFDGARDMWRAYSDMREANYIGSDKYFHARGNYDAAKRGPGGVWAAEAISDARENIQRFFGHGAEDSLADQAANEWGRSGKDPNHFRPAGLPEKY

SEQ ID NO: 29 - protein, 432 aa, Homo sapiens
MSDKLPYKVADIGLAAWGRKALDIAENEMPGLMRMRERYSASKPLKGARIAGCLHMTVETAVLIETLVTLGAEVQWSSCNIFSTQDHAAAAIAKAGIPVYAWKGETDEEYLWCIEQTLYFKDGPLNMILDDGGDLTNLIHTKYPQLLPGIRGISEETTTGVHNLYKMMANGILKVPAINVNDSVTKSKFDNLYGCRESLIDGIKRATDVMIAGKVAVVAGYGDVGKGCAQALRGFGARVIITEIDPINALQAAMEGYEVTTMDEACQEGNIFVTTTGCIDIILGRHFEQMKDDAIVCNIGHFDVEIDVKWLNENAVEKVNIKPQVDRYRLKNGRRIILLAEGRLVNLGCAMGHPSFVMSNSFTNQVMAQIELWTHPDKYPVGVHFLPKKLDEAVAEAHLGKLNVKLTKLTEKQAQYLGMSCDGPFKPDHYRY

SEQ ID NO: 30 - protein, 472 aa, Homo sapiens
MATKCGNCGPGYSTPLEAMKGPREEIVYLPCIYRNTGTEAPDYLATVDVDPKSPQYCQVIHRLPMPNLKDELHHSGWNTCSSCFGDSTKSRTKLVLPSLISSRIYVVDVGSEPRAPKLHKVIEPKDIHAKCELAFLHTSHCLASGEVMISSLGDVKGNGKGGFVLLDGETFEVKGTWERPGGAAPLGYDFWYQPRHNVMISTEWAAPNVLRDGFNPADVEAGLYGSHLYVWDWQRHEIVQTLSLKDGLIPLEIRFLHNPDAAQGFVGCALSSTIQRFYKNEGGTWSVEKVIQVPPKKVKGWLLPEMPGLITDILLSLDDRFLYFSNWLHGDLRQYDISDPQRPRLTGQLFLGGSIVKGGPVQVLEDEELKSQPEPLVVKGKRVAGGPQMIQLSLDGKRLYITTSLYSAWDKQFYPDLIREGSVMLQVDVDTVKGGLKLNPNFLVDFGKEPLGPALAHELRYPGGDCSSDIWI

SEQ ID NO: 31 - protein, 760 aa, Homo sapiens
MKTWVKIVFGVATSAVLALLVMCIVLRPSRVHNSEENTMRALTLKDILNGTFSYKTFFPNWISGQEYLHQSADNNIVLYNIETGQSYTILSNRTMKSVNASNYGLSPDRQFVYLESDYSKLWRYSYTATYYIYDLSNGEFVRGNELPRPIQYLCWSPVGSKLAYVYQNNIYLKQRPGDPPFQITFNGRENKIFNGIPDWVYEEEMLATKYALWWSPNGKFLAYAEFNDTDIPVIAYSYYGDEQYPRTINIPYPKAGAKNPVVRIFIIDTTYPAYVGPQEVPVPAMIASSDYYFSWLTWVTDERVCLQWLKRVQNVSVLSICDFREDWQTWDCPKTQEHIEESRTGWAGGFFVSTPVFSYDAISYYKIFSDKDGYKHIHYIKDTVENAIQITSGKWEAINIFRVTQDSLFYSSNEFEEYPGRRNIYRISIGSYPPSKKCVTCHLRKERCQYYTASFSDYAKYYALVCYGPGIPISTLHDGRTDQEIKILEENKELENALKNIQLPKEEIKKLEVDEITLWYKMILPPQFDRSKKYPLLIQVYGGPCSQSVRSVFAVNWISYLASKEGMVIALVDGRGTAFQGDKLLYAVYRKLGVYEVEDQITAVRKFIEMGFIDEKRIAIWGWSYGGYVSSLALASGTGLFKCGIAVAPVSSWEYYASVYTERFMGLPTKDDNLEHYKNSTVMARAEYFRNVDYLLIHGTADDNVHFQNSAQIAKALVNAQVDFQAMWYSDQNHGLSGLSTNHLYTHMTHFLKQCFSLSD

SEQ ID NO: 32 - protein, 376 aa, Homo sapiens
MDVLAEANGTFALNLLKTLGKDNSKNVFFSPMSMSCALAMVYMGAKGNTAAQMAQILSFNKSGGGGDIHQGFQSLLTEVNKTGTQYLLRMANRLFGEKSCDFLSSFRDSCQKFYQAEMEELDFISAVEKSRKHINTWVAEKTEGKIAELLSPGSVDPLTRLVLVNAVYFRGNWDEQFDKENTEERLFKVSKNEEKPVQMMFKQSTFKKTYIGEIFTQILVLPYVGKELNMIIMLPDETTDLRTVEKELTYEKFVEWTRLDMMDEEEVEVSLPRFKLEESYDMESVLRNLGMTDAFELGKADFSGMSQTDLSLSKVVHKSFVEVNEEGTEAAAATAAIMMMRCARFVPRFCADHPFLFFIQHSKTNGILFCGRFSSP

SEQ ID NO: 33 - protein, 331 aa, Homo sapiens
MENPSPAAALGKALCALLLATLGAAGQPLGGESICSARALAKYSITFTGKWSQTAFPKQYPLFRPPAQWSSLLGAAHSSDYSMWRKNQYVSNGLRDFAERGEAWALMKEIEAAGEALQSVHEVFSAPAVPSGTGQTSAELEVQRRHSLVSFVVRIVPSPDWFVGVDSLDLCDGDRWREQAALDLYPYDAGTDSGFTFSSPNFATIPQDTVTEITSSSPSHPANSFYYPRLKALPPIARVTLVRLRQSPRAFIPPAPVLPSRDNEIVDSASVPETPLDCEVSLWSSWGLCGGHCGRLGTKSRTRYVRVQPANNGSPCPELEEEAECVPDNCV

SEQ ID NO: 34 - protein, 536 aa, Homo sapiens
MGSNKSKPKDASQRRRSLEPAENVHGAGGGAFPASQTPSKPASADGHRGPSAAFAPAAAEPKLFGGFNSSDTVTSPQRAGPLAGGVTTFVALYDYESRTETDLSFKKGERLQIVNNTEGDWWLAHSLSTGQTGYIPSNYVAPSDSIQAEEWYFGKITRRESERLLLNAENPRGTFLVRESETTKGAYCLSVSDFDNAKGLNVKHYKIRKLDSGGFYITSRTQFNSLQQLVAYYSKHADGLCHRLTTVCPTSKPQTQGLAKDAWEIPRESLRLEVKLGQGCFGEVWMGTWNGTTRVAIKTLKPGTMSPEAFLQEAQVMKKLRHEKLVQLYAVVSEEPIYIVTEYMSKGSLLDFLKGETGKYLRLPQLVDMAAQIASGMAYVERMNYVHRDLRAANILVGENLVCKVADFGLARLIEDNEYTARQGAKFPIKWTAPEAALYGRFTIKSDVWSFGILLTELTTKGRVPYPGMVNREVLDQVERGYRMPCPPECPESLHDLMCQCWRKEPEERPTFEYLQAFLEDYFTSTEPQYQPGENL

SEQ ID NO: 35 - protein, 739 aa, Homo sapiens
MPSPRPVLLRGARAALLLLLPPRLLARPSLLLRRSLSAASCPPISLPAAASRSSMDGAGAEEVLAPLRLAVRQQGDLVRKLKEDKAPQVDVDKAVAELKARKRVLEAKELALQPKDDIVDRAKMEDTLKRRFFYDQAFAIYGGVSGLYDFGPVGCALKNNIIQTWRQHFIQEEQILEIDCTMLTPEPVLKTSGHVDKFADFMVKDVKNGECFRADHLLKAHLQKLMSDKKCSVEKKSEMESVLAQLDNYGQQELADLFVNYNVKSPITGNDLSPPVSFNLMFKTFIGPGGNMPGYLRPETAQGIFLNFKRLLEFNQGKLPFAAAQIGNSFRNEISPRSGLIRVREFTMAEIEHFVDPSEKDHPKFQNVADLHLYLYSAKAQVSGQSARKMRLGDAVEQGVINNTVLGYFIGRIYLYLTKVGISPDKLRFRQHMENEMAHYACDCWDAESKTSYGWIEIVGCADRSCYDLSCHARATKVPLVAEKPLKEPKTVNVVQFEPSKGAIGKAYKKDAKLVMEYLAICDECYITEMEMLLNEKGEFTIETEGKTFQLTKDMINVKRFQKTLYVEEVVPNVIEPSFGLGRIMYTVFEHTFHVREGDEQRTFFSFPAVVAPFKCSVLPLSQNQEFMPFVKELSEALTRHGVSHKVDDSSGSIGRRYARTDEIGVAFGVTIDFDTVNKTPHTATLRDRDSMRQIRAEISELPSIVQDLANGNITWADVEARYPLFEGQETGKKETIEE

SEQ ID NO: 36 - protein, 207 aa, Homo sapiens
MAPFEPLASGILLLLWLIAPSRACTCVPPHPQTAFCNSDLVIRAKFVGTPEVNQTTLYQRYEIKMTKMYKGFQALGDAADIRFVYTPAMESVCGYFHRSHNRSEEFLIAGKLQDGLLHITTCSFVAPWNSLSLAQRRGFTKTYTVGCEECTVFPCLSIPCKLQSGTHCLWTDQLLQGSEKGFQSRHLACLPREPGLCTWQSLRSQIA

SEQ ID NO: 37 - protein, 698 aa, Homo sapiens
MRLAVGALLVCAVLGLCLAVPDKTVRWCAVSEHEATKCQSFRDHMKSVIPSDGPSVACVKKASYLDCIRAIAANEADAVTLDAGLVYDAYLAPNNLKPVVAEFYGSKEDPQTFYYAVAVVKKDSGFQMNQLRGKKSCHTGLGRSAGWNIPIGLLYCDLPEPRKPLEKAVANFFSGSCAPCADGTDFPQLCQLCPGCGCSTLNQYFGYSGAFKCLKDGAGDVAFVKHSTIFENLANKADRDQYELLCLDNTRKPVDEYKDCHLAQVPSHTVVARSMGGKEDLIWELLNQAQEHFGKDKSKEFQLFSSPHGKDLLFKDSAHGFLKVPPRMDAKMYLGYEYVTAIRNLREGTCPEAPTDECKPVKWCALSHHERLKCDEWSVNSVGKIECVSAETTEDCIAKIMNGEADAMSLDGGFVYIAGKCGLVPVLAENYNKSDNCEDTPEAGYFAIAVVKKSASDLTWDNLKGKKSCHTAVGRTAGWNIPMGLLYNKINHCRFDEFFSEGCAPGSKKDSSLCKLCMGSGLNLCEPNNKEGYYGYTGAFRCLVEKGDVAFVKHQTVPQNTGGKNPDPWAKNLNEKDYELLCLDGTRKPVEEYANCHLARAPNHAVVTRKDKEACVHKILRQQQHLFGSNVTDCSGNFCLFRSETKDLLFRDDTVCLAKLHDRNTYEKYLGEEYVKAVGNLRKCSTSSLLEACTFRRP

(19) United States
(12) Patent Application Publication
(10) Pub. No.: US 2017/0176441 A1
(43) Pub. Date: Jun. 22, 2017

(54) PROTEIN BIOMARKER PROFILES FOR DETECTING COLORECTAL TUMORS

(71) Applicant: Applied Proteomics, Inc., San Diego, CA (US)

(72) Inventors: John Blume, Bellingham, WA (US); Ryan Benz, Huntington Beach, CA (US); Lisa Croner, San Diego, CA (US); Roslyn Dillon, Cardiff, CA (US); Jeffrey Jones, Glendale, CA (US); Athit Kao, San Marcos, CA (US); Heather Skor, San Diego, CA (US); Bruce Wilcox, Harrisonburg, VA (US)

(21) Appl. No.: 15/300,241

(22) PCT Filed: Mar. 27, 2015
(86) PCT No.: PCT/US15/23187

§ 371 Date: Sep. 28, 2016

Related U.S. Application Data
(60) Provisional application No. 62/107,265, filed on Jan. 23, 2015.
(60) Provisional application No. 62/005,835, filed on May 30, 2014.
(60) Provisional application No. 61/972,153, filed on Mar. 28, 2014.

(51) Int. Cl. G01N 33/574

(52) U.S. Cl. CPC G01N 33/57419

(57) ABSTRACT

Disclosed herein are panels related to the diagnosis or recognition of colon and colorectal cancer in a subject. The disclosed panels and related methods are used to predict or assess colon tumor status in a patient. They can be used to determine nature of tumor, recurrence, or patient response to treatments. Some embodiments of the methods include generating a report for clinical management.

### CROSS-REFERENCE TO RELATED APPLICATIONS

The present application claims the benefit of priority to U.S. Provisional Ser. No. 61/972,153, filed Mar. 28, 2014, which is hereby incorporated by reference in its entirety; the present application claims the benefit of priority to U.S. Provisional Ser. No. 62/005,835, filed May 30, 2014, which is hereby incorporated by reference in its entirety; and the present application claims the benefit of priority to U.S. Provisional Ser. No. 62/107,265, filed Jan. 23, 2015, which is hereby incorporated by reference in its entirety.

### BACKGROUND OF THE INVENTION

Colorectal cancer (CRC) can result from uncontrolled cell growth in the colon or rectum (parts of the large intestine), or in the appendix. CRC can develop from a colon polyp. A colon polyp typically comprises a benign clump of cells that forms on the lining of the large intestine or rectum. While many colon polyps are non-malignant, a polyp can develop into an adenoma. Colorectal adenomas can then grow into advanced colorectal adenomas, which can then develop into CRC. CRC is the third most commonly diagnosed cancer in the world, with approximately 1.23 million new diagnosed cases and 608,000 deaths by CRC in 2008 alone. In the developed world, about 33% of patients with CRC eventually die from the disease. Survival can be related to stage of the cancer upon detection. For example, survival rates for early stage detection can be about 5 times that of late stage cancers. Early diagnosis of CRC can have the potential to reduce CRC deaths by 60%. Stage I patients have a survival rate of ^{˜}85%, while the 5-year survival rate drops to ^{˜}65-75% in stage II patients and to 35-50% in stage III patients.

The most common non-invasive test for colorectal cancer is the fecal occult blood test (“FOBT”). Unfortunately, in addition to its high false-positive rate, the sensitivity of the FOBT remains around 50% and may have less sensitivity for detection of early stage CRC. Numerous serum markers, such as carcinoembryonic antigen (“CEA”), carbohydrate antigen 19-9, and lipid-associated sialic acid, have been investigated in colorectal cancer. However, their low sensitivity has induced the American Society of Clinical Oncology to state that none can be recommended for screening and diagnosis, and that their use should be limited to postsurgery surveillance.

Colonoscopy and sigmoidoscopy remain the gold standard for detecting colon cancer. However, the highly invasive nature and the expense of these exams contribute to low acceptance from the population. Furthermore, such highly invasive procedures can potentially expose the subjects to risk of infection.

### SUMMARY OF THE INVENTION

Provided herein are methods, compositions, kits, computer readable media, and systems for the diagnosis and/or treatment of at least one of advanced colorectal adenoma and colorectal cancer. For example, provided herein are biomarker panels and assays useful for the diagnosis and/or treatment of at least one of advanced colorectal adenoma and colorectal cancer.

For example, provided herein are methods of treating at least one of a colorectal cancer and advanced colorectal adenoma in a subject, comprising: (a) measuring a biomarker panel in a biological sample obtained from the subject, wherein the biomarker panel comprises at least two biomarkers selected from the group consisting of A1AG1, A1AT, AACT, AMY2B, ANXA1, APOA1, CAH1, CATD, CEAM3, CLUS, CTNB1, CO3, CO9, CRP, CSF1, DPP4, ECH1, FHL1, FIBB, FIBG, FRIL, GELS, HPT, OSTP, PRDX1, SAA1, SBP1, SEPR, SPB6, SPON2, SYG, TIMP1, and TRFE; (b) detecting a presence or absence of colorectal cancer and/or advanced colorectal adenoma in the subject based upon the measuring; and (c) treating the colorectal cancer in the subject based upon the detecting.

Provided herein are methods of treating at least one of a colorectal cancer and advanced colorectal adenoma in a subject, comprising: (a) measuring a biomarker panel in a biological sample obtained from the subject, wherein the biomarker panel comprises at least two biomarkers selected from the group consisting of A1AG1, A1AT, AACT, APOA1, CATD, CEAM3, CLUS, CO3, CO9, CRP, FIBB, FIBG, GELS, OSTP, PRDX1, SAA1, SBP1, and SEPR; (b) detecting a presence or absence of colorectal cancer and/or advanced colorectal adenoma in the subject based upon the measuring; and (c) treating the colorectal cancer in the subject based upon the detecting.

Also provided herein are methods of diagnosing at least one of a colorectal cancer and advanced colorectal adenoma in a subject, comprising: (a) measuring a biomarker panel in a biological sample obtained from the subject, wherein the biomarker panel comprises at least two biomarkers selected from the group consisting of A1AG1, A1AT, AACT, AMY2B, ANXA1, APOA1, CAH1, CATD, CEAM3, CLUS, CTNB1, CO3, CO9, CRP, CSF1, DPP4, ECH1, FHL1, FIBB, FIBG, FRIL, GELS, HPT, OSTP, PRDX1, SAA1, SBP1, SEPR, SPB6, SPON2, SYG, TIMP1, and TRFE; (b) detecting a presence or absence of at least one of colorectal cancer and advanced colorectal adenoma in the subject based upon the measuring; and (c) recommending to the subject at least one of a colonoscopy, sigmoidoscopy, and tissue biopsy in the subject based upon the detecting.

In another example, provided herein are methods of diagnosing at least one of a colorectal cancer and advanced colorectal adenoma in a subject or categorizing the colorectal status of an individual comprising: (a) measuring a biomarker panel in a biological sample obtained from the subject, wherein the biomarker panel comprises at least two biomarkers selected from the group consisting of A1AG1, A1AT, AACT, APOA1, CATD, CEAM3, CLUS, CO3, CO9, CRP, FIBB, FIBG, GELS, OSTP, PRDX1, SAA1, SBP1, and SEPR; (b) detecting a presence or absence of at least one of colorectal cancer and advanced colorectal adenoma in the subject based upon the measuring; and (c) recommending to the subject at least one of a colonoscopy, sigmoidoscopy, and tissue biopsy in the subject based upon the detecting.

In another example, provided herein are methods of diagnosing at least one of a colorectal cancer and advanced colorectal adenoma in a subject or categorizing the colorectal status of an individual comprising: (a) measuring a biomarker panel in a biological sample obtained from the subject, wherein the biomarker panel comprises at least two biomarkers selected from the group consisting of A1AG1, A1AT, CATD, CEA, CO9, OSTP, and SEPR; (b) detecting a presence or absence of at least one of colorectal cancer and advanced colorectal adenoma in the subject based upon the measuring; and (c) recommending to the subject at least one of a colonoscopy, sigmoidoscopy, and tissue biopsy in the subject based upon the detecting.

In another example, provided herein are methods of diagnosing at least one of a colorectal cancer and advanced colorectal adenoma in a subject or categorizing the colorectal status of an individual comprising: (a) measuring a biomarker panel in a biological sample obtained from the subject, wherein the biomarker panel comprises at least two biomarkers selected from the group consisting of A1AG1, A1AT, APOA1, CATD, CEA, CLUS, CO3, CO9, FGB, FIBG, GELS, PRDX1, SBP1, and SEPR; (b) detecting a presence or absence of at least one of colorectal cancer and advanced colorectal adenoma in the subject based upon the measuring; and (c) recommending to the subject at least one of a colonoscopy, sigmoidoscopy, and tissue biopsy in the subject based upon the detecting.

In another example, provided herein are methods of diagnosing at least one of a colorectal cancer and advanced colorectal adenoma in a subject or categorizing the colorectal status of an individual comprising: (a) measuring a biomarker panel in a biological sample obtained from the subject, wherein the biomarker panel comprises at least two biomarkers selected from the group consisting of A1AG1, A1AT, CATD, CEA, CO9, and SEPR; (b) detecting a presence or absence of at least one of colorectal cancer and advanced colorectal adenoma in the subject based upon the measuring; and (c) recommending to the subject at least one of a colonoscopy, sigmoidoscopy, and tissue biopsy in the subject based upon the detecting.

In another example, provided herein are methods of diagnosing at least one of a colorectal cancer and advanced colorectal adenoma in a subject or categorizing the colorectal status of an individual comprising: (a) measuring a biomarker panel in a biological sample obtained from the subject, wherein the biomarker panel comprises at least two biomarkers selected from the group consisting of A1AG1, A1AT, AACT, CATD, CEA, CO9, CRP, GELS, SAA1, and SEPR; (b) detecting a presence or absence of at least one of colorectal cancer and advanced colorectal adenoma in the subject based upon the measuring; and (c) recommending to the subject at least one of a colonoscopy, sigmoidoscopy, and tissue biopsy in the subject based upon the detecting.

In another example, provided herein are methods of diagnosing at least one of a colorectal cancer and advanced colorectal adenoma in a subject or categorizing the colorectal status of an individual comprising: (a) measuring a biomarker panel in a biological sample obtained from the subject, wherein the biomarker panel comprises at least two biomarkers selected from the group consisting of CATD, CEA, CO3, CO9, GELS, and SEPR; (b) detecting a presence or absence of at least one of colorectal cancer and advanced colorectal adenoma in the subject based upon the measuring; and (c) recommending to the subject at least one of a colonoscopy, sigmoidoscopy, and tissue biopsy in the subject based upon the detecting.

In another example, provided herein are methods of diagnosing at least one of a colorectal cancer and advanced colorectal adenoma in a subject or categorizing the colorectal status of an individual comprising: (a) measuring a biomarker panel in a biological sample obtained from the subject, wherein the biomarker panel comprises at least two biomarkers selected from the group consisting of CATD, CEA, CO9, and SEPR; (b) detecting a presence or absence of at least one of colorectal cancer and advanced colorectal adenoma in the subject based upon the measuring; and (c) recommending to the subject at least one of a colonoscopy, sigmoidoscopy, and tissue biopsy in the subject based upon the detecting.

Also provide herein are methods for monitoring an individual's response to a colorectal cancer treatment regimen administered to said individual, comprising comparing accumulation levels of a panel of proteins in a blood sample obtained from said individual at a first time period to accumulation levels of a panel of proteins in a blood sample obtained from said individual at a second time period, wherein the biomarker panel comprises A1AG1, A1AT, CATD, CEA, CO9, OSTP, and SEPR.

Also provide herein are methods for monitoring an individual's response to a colorectal cancer treatment regimen administered to said individual, comprising comparing accumulation levels of a panel of proteins in a blood sample obtained from said individual at a first time period to accumulation levels of a panel of proteins in a blood sample obtained from said individual at a second time period, wherein the biomarker panel comprises A1AG1, A1AT, APOA1, CATD, CEA, CLUS, CO3, CO9, FGB, FIBG, GELS, PRDX1, SBP1, and SEPR.

Also provide herein are methods for monitoring an individual's response to a colorectal cancer treatment regimen administered to said individual, comprising comparing accumulation levels of a panel of proteins in a blood sample obtained from said individual at a first time period to accumulation levels of a panel of proteins in a blood sample obtained from said individual at a second time period, wherein the biomarker panel comprises A1AG1, A1AT, CATD, CEA, CO9, and SEPR.

Also provide herein are methods for monitoring an individual's response to a colorectal cancer treatment regimen administered to said individual, comprising comparing accumulation levels of a panel of proteins in a blood sample obtained from said individual at a first time period to accumulation levels of a panel of proteins in a blood sample obtained from said individual at a second time period, wherein the biomarker panel comprises A1AG1, A1AT, AACT, CATD, CEA, CO9, CRP, GELS, SAA1, and SEPR.

Also provide herein are methods for monitoring an individual's response to a colorectal cancer treatment regimen administered to said individual, comprising comparing accumulation levels of a panel of proteins in a blood sample obtained from said individual at a first time period to accumulation levels of a panel of proteins in a blood sample obtained from said individual at a second time period, wherein the biomarker panel comprises CATD, CEA, CO3, CO9, GELS, and SEPR.

Also provide herein are methods for monitoring an individual's response to a colorectal cancer treatment regimen administered to said individual, comprising comparing accumulation levels of a panel of proteins in a blood sample obtained from said individual at a first time period to accumulation levels of a panel of proteins in a blood sample obtained from said individual at a second time period, wherein the biomarker panel comprises CATD, CEA, CO9, and SEPR.

Also provided herein are methods, comprising: (a) obtaining data comprising a measurement of a biomarker panel in a biological sample obtained from a subject, wherein the biomarker panel comprises at least two biomarkers selected from the group consisting of A1AG1, A1AT, AACT, AMY2B, ANXA1, APOA1, CAH1, CATD, CEAM3, CLUS, CTNB1, CO3, CO9, CRP, CSF1, DPP4, ECH1, FHL1, FIBB, FIBG, FRIL, GELS, HPT, OSTP, PRDX1, SAA1, SBP1, SEPR, SPB6, SPON2, SYG, TIMP1, and TRFE; (b) generating a subject-specific profile of the biomarker panel based upon the measurement data; (c) comparing the subject-specific profile of the biomarker panel to a reference profile of the biomarker panel; and (d) determining a likelihood of at least one of advanced colorectal adenoma and colorectal cancer based upon (c).

In another example, provided herein are methods, comprising: (a) obtaining data comprising a measurement of a biomarker panel in a biological sample obtained from a subject, wherein the biomarker panel comprises at least two biomarkers selected from the group consisting of A1AG1, A1AT, AACT, APOA1, CATD, CEAM3, CLUS, CO3, CO9, CRP, FIBB, FIBG, GELS, OSTP, PRDX1, SAA1, SBP1, and SEPR; (b) generating a subject-specific profile of the biomarker panel based upon the measurement data; (c) comparing the subject-specific profile of the biomarker panel to a reference profile of the biomarker panel; and (d) determining a likelihood of at least one of advanced colorectal adenoma and colorectal cancer based upon (c).

In some embodiments, any of the foregoing methods comprise detecting a presence or absence of advanced colorectal adenoma in the subject. In some embodiments, the advanced colorectal adenoma comprises a dimension that is greater than or equal to 1 centimeter. In some embodiments, the advanced colorectal adenoma is of villous character. In some embodiments, the method comprises detecting a presence or absence of the advanced colorectal adenoma with a sensitivity that is greater than 70%. In some embodiments, the method comprises detecting a presence or absence of the advanced colorectal adenoma with a sensitivity that is greater than 75%, 80%, 85%, 90%, or 95%. In some embodiments, the method further comprises removing the advanced colorectal adenoma from the subject, thereby preventing development of colorectal cancer in the subject. In some embodiments, the biomarker panel comprises CATD and FUCO. In some embodiments, the method comprises detecting a presence of an advanced colorectal adenoma in the subject if a level of at least one of CATD and FUCO in the biological sample obtained from the subject differs from a negative control reference level of the least one of CATD and FUCO by at least 10%. In some embodiments, the method comprises detecting a presence of an advanced colorectal adenoma in the subject if a level of at least one of CATD and FUCO in the biological sample obtained from the subject differs from a positive control reference level of the least one of CATD and FUCO by less than 10%. In some embodiments, the biomarker panel comprises three biomarkers. In some embodiments, the three biomarkers are CATD, CATS, and FUCO. In some embodiments, the method comprises detecting a presence of an advanced colorectal adenoma in the subject if a level of at least one of CATD, CATS, and FUCO in the biological sample obtained from the subject differs from a negative control reference level of the least one of CATD, CATS, and FUCO by at least 10%. In some embodiments, the method comprises detecting a presence of an advanced colorectal adenoma in the subject if a level of at least one of CATD, CATS, and FUCO in the biological sample obtained from the subject differs from a positive control reference level of the least one of CATD, CATS, and FUCO by less than 10%. In some embodiments, the three biomarkers are CATD, FUCO, and FIBB. In some embodiments, the method comprises detecting a presence of an advanced colorectal adenoma in the subject if a level of at least one of CATD, FUCO, and FIBB in the biological sample obtained from the subject differs from a negative control reference level of the least one of CATD, FUCO, and FIBB by at least 10%. In some embodiments, the method comprises detecting a presence of an advanced colorectal adenoma in the subject if a level of at least one of CATD, FUCO, and FIBB in the biological sample obtained from the subject differs from a positive control reference level of the least one of CATD, FUCO, and FIBB by less than 10%. In some embodiments, the three biomarkers are CATD, FUCO, and SAHH. In some embodiments, the method comprises detecting a presence of an advanced colorectal adenoma in the subject if a level of at least one of CATD, FUCO, and SAHH in the biological sample obtained from the subject differs from a negative control reference level of the least one of CATD, FUCO, and SAHH by at least 10%. In some embodiments, the method comprises detecting a presence of an advanced colorectal adenoma in the subject if a level of at least one of CATD, FUCO, and SAHH in the biological sample obtained from the subject differs from a positive control reference level of the least one of CATD, FUCO, and SAHH by less than 10%. In some embodiments, the biomarker panel comprises no more than three biomarkers. In some embodiments, the biomarker panel comprises four biomarkers. In some embodiments, the four biomarkers are CATD, FIBB, FUCO, and SAHH. In some embodiments, the method comprises detecting a presence of an advanced colorectal adenoma in the subject if a level of at least one of CATD, FIBB, FUCO, and SAHH in the biological sample obtained from the subject differs from a negative control reference level of the least one of CATD, FIBB, FUCO, and SAHH by at least 10%. In some embodiments, the method comprises detecting a presence of an advanced colorectal adenoma in the subject if a level of at least one of CATD, FIBB, FUCO, and SAHH in the biological sample obtained from the subject differs from a positive control reference level of the least one of CATD, FIBB, FUCO, and SAHH by less than 10%.

In some embodiments, a method described herein comprises detecting a presence or absence of colorectal cancer in the subject. In some embodiments, the biomarker panel comprises CO9 and GELS. In some embodiments, the method comprises detecting a presence of colorectal cancer in the subject if a level of at least one of CO9 and GELS in the biological sample obtained from the subject differs from a negative control reference level of the least one of CO9 and GELS by at least 10%. In some embodiments, the method comprises detecting a presence of colorectal cancer in the subject if a level of at least one of CO9 and GELS in the biological sample obtained from the subject differs from a positive control reference level of the least one of CO9 and GELS by less than 10%.

In some embodiments, the biomarker panel comprises at least three biomarkers. In some embodiments, the at least three biomarkers comprise AACT, CO9, and SYG. In some embodiments, the method comprises detecting a presence of colorectal cancer in the subject if a level of at least one of AACT, CO9, and SYG in the biological sample obtained from the subject differs from a negative control reference level of the least one of AACT, CO9, and SYG by at least 10%. In some embodiments, the method comprises detecting a presence of colorectal cancer in the subject if a level of at least one of AACT, CO9, and SYG in the biological sample obtained from the subject differs from a positive control reference level of the least one of AACT, CO9, and SYG by less than 10%. In some embodiments, the biomarker panel comprises CRP and TIMP1. In some embodiments, the method comprises detecting a presence of colorectal cancer in the subject if a level of at least one of CRP and TIMP1 in the biological sample obtained from the subject differs from a negative control reference level of the least one of CRP and TIMP1 by at least 10%. In some embodiments, the method comprises detecting a presence of colorectal cancer in the subject if a level of at least one of CRP and TIMP1 in the biological sample obtained from the subject differs from a positive control reference level of the least one of CRP and TIMP1 by less than 10%.

In some embodiments, the biomarker panel comprises at least four biomarkers. In some embodiments, the at least four biomarkers comprise CO9, GELS, PRDX1, and CATD. In some embodiments, the method comprises detecting a presence of colorectal cancer in the subject if a level of at least one of CO9, GELS, PRDX1, and CATD in the biological sample obtained from the subject differs from a negative control reference level of the least one of CO9, GELS, PRDX1, and CATD by at least 10%. In some embodiments, the method comprises detecting a presence of colorectal cancer in the subject if a level of at least one of CO9, GELS, PRDX1, and CATD in the biological sample obtained from the subject differs from a positive control reference level of the least one of CO9, GELS, PRDX1, and CATD by less than 10%. In some embodiments, the at least four biomarkers comprise A1AT, APOA1, FIBB, and CEAM3. In some embodiments, the method comprises detecting a presence of colorectal cancer in the subject if a level of at least one of A1AT, APOA1, FIBB, and CEAM3 in the biological sample obtained from the subject differs from a negative control reference level of the least one of A1AT, APOA1, FIBB, and CEAM3 by at least 10%. In some embodiments, the method comprises detecting a presence of colorectal cancer in the subject if a level of at least one of A1AT, APOA1, FIBB, and CEAM3 in the biological sample obtained from the subject differs from a positive control reference level of the least one of A1AT, APOA1, FIBB, and CEAM3 by less than 10%. In some embodiments, the at least four biomarkers comprise CAH1, CRP, FIBG, and CTNB1. In some embodiments, the method comprises detecting a presence of colorectal cancer in the subject if a level of at least one of CAH1, CRP, FIBG, and CTNB1 in the biological sample obtained from the subject differs from a negative control reference level of the least one of CAH1, CRP, FIBG, and CTNB1 by at least 10%. In some embodiments, the method comprises detecting a presence of colorectal cancer in the subject if a level of at least one of CAH1, CRP, FIBG, and CTNB1 in the biological sample obtained from the subject differs from a positive control reference level of the least one of CAH1, CRP, FIBG, and CTNB1 by less than 10%. In some embodiments, the at least four biomarkers comprise A1AG1, A1AT, CO9, and GELS. In some embodiments, the method comprises detecting a presence of colorectal cancer in the subject if a level of at least one of A1AG1, A1AT, CO9, and GELS in the biological sample obtained from the subject differs from a negative control reference level of the least one of A1AG1, A1AT, CO9, and GELS by at least 10%. In some embodiments, the method comprises detecting a presence of colorectal cancer in the subject if a level of at least one of A1AG1, A1AT, CO9, and GELS in the biological sample obtained from the subject differs from a positive control reference level of the least one of A1AG1, A1AT, CO9, and GELS by less than 10%.

In some embodiments, the biomarker panel comprises 13 biomarkers. In some embodiments, the 13 biomarkers are A1AG1, A1AT, AACT, ANXA1, APOA1, CO9, CRP, CSF1, FHL1, FIBG, GELS, HPT, and SAM. In some embodiments, the method comprises detecting a presence of colorectal cancer in the subject if a level of at least one of A1AG1, A1AT, AACT, ANXA1, APOA1, CO9, CRP, CSF1, FHL1, FIBG, GELS, HPT, and SAM in the biological sample obtained from the subject differs from a negative control reference level of the least one of A1AG1, A1AT, AACT, ANXA1, APOA1, CO9, CRP, CSF1, FHL1, FIBG, GELS, HPT, and SAM by at least 10%. In some embodiments, the method comprises detecting a presence of colorectal cancer in the subject if a level of at least one of A1AG1, A1AT, AACT, ANXA1, APOA1, CO9, CRP, CSF1, FHL1, FIBG, GELS, HPT, and SAM in the biological sample obtained from the subject differs from a positive control reference level of the least one of A1AG1, A1AT, AACT, ANXA1, APOA1, CO9, CRP, CSF1, FHL1, FIBG, GELS, HPT, and SAM by less than 10%. In some embodiments, the 13 biomarkers are A1AG1, A1AT, AMY2B, CLUS, CO9, ECH1, FRIL, GELS, OSTP, SBP1, SEPR, SPON2, and TIMP1. In some embodiments, the method comprises detecting a presence of colorectal cancer in the subject if a level of at least one of A1AG1, A1AT, AMY2B, CLUS, CO9, ECH1, FRIL, GELS, OSTP, SBP1, SEPR, SPON2, and TIMP1 in the biological sample obtained from the subject differs from a negative control reference level of the least one of A1AG1, A1AT, AMY2B, CLUS, CO9, ECH1, FRIL, GELS, OSTP, SBP1, SEPR, SPON2, and TIMP1 by at least 10%. In some embodiments, the method comprises detecting a presence of colorectal cancer in the subject if a level of at least one of A1AG1, A1AT, AMY2B, CLUS, CO9, ECH1, FRIL, GELS, OSTP, SBP1, SEPR, SPON2, and TIMP1 in the biological sample obtained from the subject differs from a positive control reference level of the least one of A1AG1, A1AT, AMY2B, CLUS, CO9, ECH1, FRIL, GELS, OSTP, SBP1, SEPR, SPON2, and TIMP1 by less than 10%.

In some embodiments, the biomarker panel comprises at least five biomarkers in the biological sample of the subject. In some embodiments, the at least five biomarkers comprise AACT, CO3, CO9, CRP, and GELS. In some embodiments, the method comprises detecting a presence of colorectal cancer in the subject if a level of at least one of AACT, CO3, CO9, CRP, and GELS in the biological sample obtained from the subject differs from a negative control reference level of the least one of AACT, CO3, CO9, CRP, and GELS by at least 10%. In some embodiments, the method comprises detecting a presence of colorectal cancer in the subject if a level of at least one of AACT, CO3, CO9, CRP, and GELS in the biological sample obtained from the subject differs from a positive control reference level of the least one of AACT, CO3, CO9, CRP, and GELS by less than 10%. In some embodiments, the at least five biomarkers comprise A1AT, CO3, FIBG, GELS, and SPB6. In some embodiments, the method comprises detecting a presence of colorectal cancer in the subject if a level of at least one of A1AT, CO3, FIBG, GELS, and SPB6 in the biological sample obtained from the subject differs from a negative control reference level of the least one of A1AT, CO3, FIBG, GELS, and SPB6 by at least 10%. In some embodiments, the method comprises detecting a presence of colorectal cancer in the subject if a level of at least one of A1AT, CO3, FIBG, GELS, and SPB6 in the biological sample obtained from the subject differs from a positive control reference level of the least one of A1AT, CO3, FIBG, GELS, and SPB6 by less than 10%. In some embodiments, the at least five biomarkers comprise CRP, DPP4, SBP1, SEPR, and SRC. In some embodiments, the method comprises detecting a presence of colorectal cancer in the subject if a level of at least one of CRP, DPP4, SBP1, SEPR, and SRC in the biological sample obtained from the subject differs from a negative control reference level of the least one of CRP, DPP4, SBP1, SEPR, and SRC by at least 10%. In some embodiments, the method comprises detecting a presence of colorectal cancer in the subject if a level of at least one of CRP, DPP4, SBP1, SEPR, and SRC in the biological sample obtained from the subject differs from a positive control reference level of the least one of CRP, DPP4, SBP1, SEPR, and SRC by less than 10%. In some embodiments, the method comprises the subject is a male subject.

In some embodiments of any of the methods described herein, the biomarker panel comprises no more than five biomarkers. In some embodiments of any of the methods described herein, the biomarker panel does not comprise CO3-desARg, ORM, CO3, CO9, GELS, CRP, SAA2, or CEA.

Also described herein are methods of treating colorectal cancer in a subject, comprising (a) determining a first ratio of a level of a first biomarker which is APOA1 to a level of a second biomarker in a biological sample obtained from the subject; (b) detecting a presence or absence of colorectal cancer in the subject based upon the determining; and (c) treating the colorectal cancer in the subject based upon the detecting.

Also described herein are methods of treating colorectal cancer in a subject, comprising: (a) determining a first ratio of a level of a first biomarker which is APOA1 to a level of a second biomarker in a biological sample obtained from the subject; (b) detecting a presence or absence of colorectal cancer in the subject based upon the determining; and (c) recommending to the subject at least one of a colonoscopy, sigmoidoscopy, and tissue biopsy to confirm a diagnosis of colorectal cancer in the subject based upon the detecting.

Also described herein are methods, comprising obtaining data comprising a measurement of a first ratio of a level of a first biomarker which is APOA1 to a level of a second biomarker in a biological sample obtained from a subject, generating a subject-specific biomarker profile based on the measurement, and comparing the subject-specific biomarker profile to a reference profile of the first ratio. The method can further comprise determining a likelihood of colorectal cancer based upon the comparing.

In some embodiments, the second biomarker is selected from the group consisting of CO3, CO9, A1AT, and FIBG. In some embodiments, the first ratio is a ratio of APOA1 to CO3. In some embodiments, the method comprises detecting a presence of colorectal cancer in the subject if the ratio of APOA1 to CO3 differs from a negative control reference ratio of APOA1 to CO3 by at least 10%. In some embodiments, the method comprises detecting a presence of colorectal cancer in the subject if the ratio of APOA1 to CO3 differs from a positive control reference ratio of APOA1 to CO3 by less than 10%. In some embodiments, the first ratio is a ratio of APOA1 to CO9. In some embodiments, the method comprises detecting a presence of colorectal cancer in the subject if the ratio of APOA1 to CO9 differs from a negative control reference ratio of APOA1 to CO9 by at least 10%. In some embodiments, the method comprises detecting a presence of colorectal cancer in the subject if the ratio of APOA1 to CO9 differs from a positive control reference ratio of APOA1 to CO9 by less than 10%. In some embodiments, the first ratio is a ratio of A1AT to APOA1. In some embodiments, the method comprises detecting a presence of colorectal cancer in the subject if the ratio of A1AT to APOA1 differs from a negative control reference ratio of A1AT to APOA1 by at least 10%. In some embodiments, the method comprises detecting a presence of colorectal cancer in the subject if the ratio of A1AT to APOA1 differs from a positive control reference ratio of A1AT to APOA1 by less than 10%. In some embodiments, the first ratio is a ratio of APOA1 to FIBG. In some embodiments, the method comprises a presence of colorectal cancer in the subject if the ratio of APOA1 to FIBG differs from a negative control reference ratio of APOA1 to FIBG by at least 10%. In some embodiments, the method comprises detecting a presence of colorectal cancer in the subject if the ratio of APOA1 to FIBG differs from a positive control reference ratio of APOA1 to FIBG by less than 10%. In some embodiments, the method further comprises determining a second ratio of a level of the first biomarker which is APOA1 to a level of a third biomarker in the biological sample of the subject. In some embodiments, the third biomarker is selected from the group consisting of CO3, CO9, A1AT, and FIBG. In some embodiments, the first ratio is a ratio of APOA1 to CO3 and the second ratio is a ratio of APOA1 to CO9. In some embodiments, the first ratio is a ratio of A1AT to APOA1 and the second ratio is a ratio of APOA1 to FIBG. In some embodiments, the method comprises detecting a presence of colorectal cancer in the subject if at least one of: the first ratio differs from a negative control reference first ratio by at least 10%, the second ratio differs from a negative control reference second ratio by at least 10%, the first ratio differs from a positive control reference first ratio by less than 10%, and the second ratio differs from a positive control reference second ratio by less than 10%.

Provided herein are methods of treating colorectal cancer in a subject, comprising: (a) determining a ratio of a level of a first biomarker which is A1AT to a level of a second biomarker which is TRFE in a biological sample obtained from the subject; (b) detecting a presence or absence of colorectal cancer in the subject based upon the determining; and (c) treating the colorectal cancer in the subject based upon the detecting.

Provided herein are methods of treating colorectal cancer in a subject, comprising: (a) determining a ratio of a level of a first biomarker which is A1AT to a level of a second biomarker which is TRFE in a biological sample obtained from the subject; (b) detecting a presence or absence of colorectal cancer in the subject based upon the determining; and (c) recommending to the subject at least one of a colonoscopy, sigmoidoscopy, and tissue biopsy to confirm a diagnosis of colorectal cancer in the subject based upon the detecting.

Also provided herein are methods, comprising obtaining data from a biological sample obtained from a subject, wherein the data comprises a measurement of a ratio of a level of a first biomarker which is A1AT to a level of a second biomarker which is TRFE in the biological sample; generating a subject-specific biomarker profile based on the measurement, and comparing the subject-specific biomarker profile to a reference profile of the ratio. The method can further comprise determining a likelihood of colorectal cancer based upon the comparing.

In some embodiments, the subject is male. In some embodiments, the method comprises detecting a presence of colorectal cancer in the subject if the ratio differs from a negative control reference ratio by at least 10%. In some embodiments, the method comprises a presence of colorectal cancer in the subject if the ratio differs from a positive control reference ratio by less than 10%.

In some embodiments of any of the foregoing methods, the biological sample is selected from the group consisting of whole blood, serum, plasma, blood constituent, bone marrow, saliva, cheek swab, urine, stool, lymph fluid, CNS fluid, and lesion exudate. In some embodiments, the biological sample is a blood sample. In some embodiments, the blood sample is a whole blood sample. In some embodiments, the blood sample is a plasma sample. In some embodiments, the blood sample is a serum sample. In some embodiments, the subject is a human subject. In some embodiments, the subject is asymptomatic for colorectal cancer. In some embodiments, the subject is at least 30 years of age or older. In some embodiments, the subject is at least 40 years of age or older. In some embodiments, the subject is at least 50 years of age or older. In some embodiments, the method is performed at a frequency that is once per year or higher. In some embodiments, the subject has had a colonoscopy, sigmoidoscopy, or colon tissue biopsy. In some embodiments, the method comprising validating a result of the colonoscopy, sigmoidoscopy, or colon tissue biopsy based upon the result of the measuring. In some embodiments, the subject has not had a colonoscopy, sigmoidoscopy, or colon tissue biopsy. In some embodiments, the subject has one or more of: a symptom of colorectal cancer, a family history of colorectal cancer, and a risk factor for colorectal cancer. In some embodiments, the subject has a previous history of at least one of a colorectal polyp, an adenoma, and CRC. In some embodiments, the measuring comprises detecting or measuring a level of a fragment, antigen, or transition ion of the at least two biomarkers. In some embodiments, the determining a ratio of the first biomarker to the second biomarker and optionally determining a second ratio of the first biomarker to the third biomarker comprises detecting or measuring a level of a fragment, antigen, or transition ion of the first biomarker, detecting or measuring a level of a fragment, antigen, or transition ion of the second biomarker, and optionally detecting or measuring a level of a fragment, antigen, or transition ion of the third biomarker. In some embodiments, the measuring comprises use of at least one of: an immunoassay, flow cytometry assay, biochip assay, mass spectrometry assay, and HPLC assay.

Also provided herein is a computer system for detecting a presence or absence of at least one of an advanced colorectal adenoma and colorectal cancer in a subject, the computer system comprising: (a) a memory unit for receiving data comprising measurement of a biomarker panel from a biological sample of the subject, wherein the biomarker panel comprises at least two biomarkers selected from the group consisting of A1AG1, A1AT, AACT, AMY2B, ANXA1, APOA1, CAH1, CATD, CEAM3, CLUS, CTNB1, CO3, CO9, CRP, CSF1, DPP4, ECH1, FHL1, FIBB, FIBG, FRIL, GELS, HPT, OSTP, PRDX1, SAA1, SBP1, SEPR, SPB6, SPON2, SYG, TIMP1, and TRFE; (b) computer-executable instructions for analyzing the measurement data according to a method of any of the preceding claims; and (c) computer-executable instructions for determining a presence or absence of at least one of advanced colorectal adenoma and colorectal cancer in the subject based upon the analyzing. In some embodiments, the computer system further comprises computer-executable instructions to generate a report of the presence or absence of the at least one of an advanced colorectal adenoma and colorectal cancer in the subject. In some embodiments, the computer system further comprises a user interface configured to communicate or display said report to a user.

Also provided herein are computer systems for detecting a presence or absence of at least one of an advanced colorectal adenoma and colorectal cancer in a subject, the computer system comprising: (a) a memory unit for receiving data comprising measurement of a biomarker panel from a biological sample of the subject, wherein the biomarker panel comprises at least two biomarkers selected from the group consisting of A1AG1, A1AT, AACT, APOA1, CATD, CEAM3, CLUS, CO3, CO9, CRP, FIBB, FIBG, GELS, OSTP, PRDX1, SAA1, SBP1, and SEPR; (b) computer-executable instructions for analyzing the measurement data according to a method of any of the preceding claims; and (c) computer-executable instructions for determining a presence or absence of at least one of advanced colorectal adenoma and colorectal cancer in the subject based upon the analyzing. In some embodiments, the computer system further comprises computer-executable instructions to generate a report of the presence or absence of the at least one of an advanced colorectal adenoma and colorectal cancer in the subject. In some embodiments, the computer system further comprises a user interface configured to communicate or display said report to a user.

Also provided herein are computer readable media comprising: (a) computer-executable instructions for analyzing data comprising measurement of a biomarker panel from a biological sample obtained from a subject, wherein the biomarker panel comprises at least two biomarkers selected from the group consisting of A1AG1, A1AT, AACT, AMY2B, ANXA1, APOA1, CAH1, CATD, CEAM3, CLUS, CTNB1, CO3, CO9, CRP, CSF1, DPP4, ECH1, FHL1, FIBB, FIBG, FRIL, GELS, HPT, OSTP, PRDX1, SAA1, SBP1, SEPR, SPB6, SPON2, SYG, TIMP1, and TRFE; and (b) computer-executable instructions for determining a presence or absence of at least one of advanced colorectal adenoma and colorectal cancer in the subject based upon the analyzing. In some embodiments, the analyzing comprises generating a subject-specific biomarker profile of the biomarker panel based upon the measurement. In some embodiments, the analyzing comprises comparing the subject-specific biomarker profile to a reference biomarker profile.

Also provided herein are computer readable media comprising: (a) computer-executable instructions for analyzing data comprising measurement of a biomarker panel from a biological sample obtained from a subject, wherein the biomarker panel comprises at least two biomarkers selected from the group consisting of A1AG1, A1AT, AACT, APOA1, CATD, CEAM3, CLUS, CO3, CO9, CRP, FIBB, FIBG, GELS, OSTP, PRDX1, SAA1, SBP1, and SEPR; and (b) computer-executable instructions for determining a presence or absence of at least one of advanced colorectal adenoma and colorectal cancer in the subject based upon the analyzing. In some embodiments, the analyzing comprises generating a subject-specific biomarker profile of the biomarker panel based upon the measurement. In some embodiments, the analyzing comprises comparing the subject-specific biomarker profile to a reference biomarker profile.

Also provided herein are kits, comprising: (a) one or more compositions for use in measuring a biomarker panel in a biological sample obtained from a subject, wherein the biomarker panel comprises at least two biomarkers selected from the group consisting of A1AG1, A1AT, AACT, AMY2B, ANXA1, APOA1, CAH1, CATD, CEAM3, CLUS, CTNB1, CO3, CO9, CRP, CSF1, DPP4, ECH1, FHL1, FIBB, FIBG, FRIL, GELS, HPT, OSTP, PRDX1, SAA1, SBP1, SEPR, SPB6, SPON2, SYG, TIMP1, and TRFE; and (b) instructions for performing a method of any of the preceding claims. In some embodiments, the kit comprises a computer readable medium described herein.

Also provided herein are kits, comprising: (a) one or more compositions for use in measuring a biomarker panel in a biological sample obtained from a subject, wherein the biomarker panel comprises at least two biomarkers selected from the group consisting of A1AG1, A1AT, AACT, APOA1, CATD, CEAM3, CLUS, CO3, CO9, CRP, FIBB, FIBG, GELS, OSTP, PRDX1, SAA1, SBP1, and SEPR; and (b) instructions for performing a method of any of the preceding claims. In some embodiments, the kit comprises a computer readable medium described herein.

For example, such kits can consist of antibodies for an ELISA assay to assess colorectal cancer status of an individual from whom a sample is derived. In some cases, such kits include antibodies that are reactive against A1AG1, A1AT, CATD, CEA, CO9, OSTP, and SEPR. In some cases, such kits include antibodies that are reactive against A1AG1, A1AT, APOA1, CATD, CEA, CLUS, CO3, CO9, FGB, FIBG, GELS, PRDX1, SBP1, and SEPR. In some cases, such kits include antibodies that are reactive against A1AG1, A1AT, CATD, CEA, CO9, and SEPR. In some cases, such kits include antibodies that are reactive against A1AG1, A1AT, AACT, CATD, CEA, CO9, CRP, GELS, SAA1, and SEPR. In some cases, such kits include antibodies that are reactive against CATD, CEA, CO3, CO9, GELS, and SEPR. In some cases, such kits include antibodies that are reactive against CATD, CEA, CO9, and SEPR.

Also provided herein are kits, comprising a computer readable medium described herein, and instructions for use of the computer readable medium.

Panels as disclosed herein are used to direct the measurement of protein accumulation levels in patient samples. At least one patient sample is obtained and protein accumulation levels are determined for a plurality of proteins in a panel. In some cases the accumulation levels for proteins in the panel are compared to protein levels of an individual of known cancer status. The individual for comparison is in some cases an individual known to be free of the cancer assayed for. In some cases the individual is known to be positive for the cancer assayed for. In some cases the accumulation levels for proteins in the panel are compared to protein levels of a plurality of individuals of known cancer status, such as free of the cancer being assayed for. In some cases the accumulation levels for proteins in the panel are compared to protein levels of a plurality of individuals that are positive for the cancer being assayed for.

Panel protein accumulation levels are normalized in some cases, such as against the mass or volume of a sample, or against the accumulation of a non-panel protein accumulation level. Generally, measurement and calculation approaches for a sample protein panel and a control panel are similar, such that accumulation levels are freely comparable between standard protein accumulation levels and sample protein accumulation levels for a given panel.

In many cases, protein accumulation levels are compared from panel to panel rather than individually. That is, individual protein accumulation levels are measured and compared, but a determination as to the classification of a patient as likely free of the cancer assayed for or likely harboring the cancer assayed for is not based upon any single protein accumulation level discrepancy. Rather, the panel measurements as a whole are compared. A number of methods for comparing protein panel member accumulation levels are known to one of skill in the art, and are contemplated herein. For example, in a relatively simple case, for each protein in a panel, the difference in the accumulation level of a standard and a patient sample is calculated, and the panel is evaluated as resembling or not resembling the standard based upon the sum of the differences between the sample and the standard for each member of the panel. In alternate embodiments or in combination, sample and standard panels are tested using a chi-squared test or an ANOVA statistical test to identify differences between the sample and the standard in accumulation level patterns. That is, rather than comparing relative accumulation levels or in addition to comparing relative accumulation levels, panel accumulation patterns are assayed. In alternative embodiments or in combination, a classifier can be applied to the individual's measured biomarkers to classify the individual as likely free of the cancer assayed for or likely harboring the cancer assayed for. Such classifiers can be, for example, a (n−1)-dimensional hyperplane that divides an n-dimensional space (e.g. a space whose dimensions are the measurements of n biomarkers) so as to maximize the distance between the classifier and the nearest data point on either side of the classifier. Such classifiers can be generated by measuring the panel of biomarkers in patients who are known to have the cancer assayed and comparing said measurements to those from healthy individuals and using a supervised learning model, such as a support vector machine learning models, to generate the classifier. Assays are performed such that a difference in relative accumulation patters within a sample pattern, as compared to within a control panel, are indicative that a sample is or is not similar to a standard. Thus in some embodiments no single protein accumulation level is determinative of patient status. Rather, it is the panels as disclosed herein that are being compared to obtain information informative of patient status.

In many cases samples are obtained from patient blood. Alternately or in combination, samples are obtained from other patient bodily fluids such as urine or saliva. In some cases samples are obtained from patient stool. Protein accumulation levels are determined through any one of a number of methods. For example, in some cases protein accumulation levels are determined from subjecting sample proteins to an ELISA assay, such as an ELISA assay provided as a kit comprising reagents for the assay of each protein in a protein panel. In some cases the reagents comprise antibodies, such as monoclonal antibodies or polyclonal antibodies or both monoclonal and polyclonal antibodies. In some cases samples are assayed using a mass spectrometry analysis. Full polypeptides are assayed in some cases, while in alternate embodiment or in combination polypeptide fragments are assayed as representative of protein accumulation levels.

Depending upon the outcome of a panel comparison assay, a number of recommendations are available. In some cases, such as when a panel comparison indicates an absence of the cancer condition assayed for, a recommendation comprises continued monitoring, such as annual monitoring, monitoring again in two years, monitoring again in five years, or monitoring at a six month time interval. Other time intervals are contemplated. Similarly, a positive panel comparison to a healthy standard is followed by a recommendation to exercise, maintain a healthy diet, or to avoid carcinogens. In some cases a positive panel comparison to a healthy standard is followed by a recommendation to perform an independent assessment of CR health, such as through a stool sample test.

In some cases a positive panel comparison is not accompanied by a health recommendation or monitoring recommendation.

In some cases, such as when a panel comparison indicates the presence of the cancer condition assayed for, a recommendation comprises continued monitoring, such as annual or semiannual monitoring. In some cases, such as when a panel comparison indicates the presence of the cancer condition assayed for, a recommendation comprises a repetition of the assay, or performance of an independent assay, such as a stool assay, to verify the outcome. In some cases a recommendation comprises a colonoscopy or sigmoidoscopy to obtain ‘gold-standard’ verification of the panel comparison results.

In some cases a recommendation comprises administration or initiation of a therapeutic regimen to treat, ameliorate the symptoms of, retard the progression of, halt the progression of, trigger the remission of, or eliminate the cancer condition assayed for. Such agents include but are not limited to 5FU, capecitabine, oxaliplatin, and bevacizumab, alone or in combination. In some cases when a panel comparison indicates the presence of the cancer condition assayed for, a recommendation comprises continued monitoring in combination with a treatment regimen as discussed above, such that the efficacy of such a treatment is monitored over time such as so determine whether the treatment regimen is predicted to demonstrate efficacy towards the treatment goal.

A number of treatment regimens are contemplated herein and known to one of skill in the art, such as chemotherapy, administration of a biologic therapeutic agent, and surgical intervention such as low anterior resection or abdominoperineal resection, or ostomy.

### Incorporation by Reference

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 depicts an exemplary computer system for implementing a method described herein.

FIG. 2 depicts discovery and validation sets for discovery and validation of protein biomarker panels.

FIGS. 3A and 3B depict discovery and validation ROC curves, respectively, obtained by assaying an exemplary biomarker panel comprising the proteins A1AG1, A1AT, AMY2B, CLUS, CO9, ECH1, FRIL, GELS, OSTP, SBP1, SEPR, SPON2, and TIMP1.

FIGS. 4A and 4B depict discovery and validation ROC curves, respectively, obtained by assaying an exemplary biomarker panel comprising the proteins CO9 and GELS.

FIGS. 5A and 5B depict discovery and validation ROC curves, respectively, obtained by assaying protein ratios of A1AT/APOA1 and APOA1/FIBG

FIGS. 6A and 6B depict discovery and validation ROC curves, respectively, obtained by assaying protein ratios of APOA1/CO3 and APOA1/CO9

FIGS. 7A and 7B depict results from a misclassification analysis to test for misclassification by sample set.

FIG. 8 depicts a validation ROC curve obtained by assaying an exemplary biomarker panel for advanced colorectal adenoma, comprising FUCO, FIBB, CATD, and SAHH.

FIG. 9 depicts a validation ROC curve obtained by assaying an exemplary biomarker panel for advanced colorectal adenoma, comprising CATD, CATS, and FUCO.

### DETAILED DESCRIPTION OF THE INVENTION

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

The practice of the present invention can employ, unless otherwise indicated, conventional techniques of immunology, biochemistry, chemistry, molecular biology, microbiology, cell biology, genomics and recombinant DNA, which are within the skill of the art. See, for example, Sambrook, Fritsch and Maniatis, MOLECULAR CLONING: A LABORATORY MANUAL, 4th edition (2012); CURRENT PROTOCOLS IN MOLECULAR BIOLOGY (F. M. Ausubel, et al. eds., (1987)); the series METHODS IN ENZYMOLOGY (Academic Press, Inc.): PCR 2: A PRACTICAL APPROACH (M. J. MacPherson, B. D. Hames and G. R. Taylor eds. (1995)), CULTURE OF ANIMAL CELLS: A MANUAL OF BASIC TECHNIQUE AND SPECIALIZED APPLICATIONS, 6th Edition (R. I. Freshney, ed. (2010), and Lange, et. al., Molecular Systems Biology Vol. 4:Article 222 (2008), which are hereby incorporated by reference.

### DEFINITIONS

As used in the specification and claims, the singular forms “a”, “an” and “the” include plural references unless the context clearly dictates otherwise. For example, the term “a sample” includes a plurality of samples, including mixtures thereof.

The terms “determining”, “measuring”, “evaluating”, “assessing,” “assaying,” and “analyzing” can be used interchangeably herein to refer to any form of measurement, and include determining if an element is present or not (for example, detection). These terms can include both quantitative and/or qualitative determinations. Assessing may be relative or absolute. “Detecting the presence of” can include determining the amount of something present, as well as determining whether it is present or absent.

The terms “panel”, “biomarker panel”, “classifier model”, and “model” can be used interchangeably herein to refer to a set of biomarkers, wherein the set of biomarkers comprises at least two biomarkers. The biomarker panel can be predictive and/or informative of a subject's health status, disease, or condition.

The terms “colorectal cancer” and “CRC” are used interchangeably herein. The term “colorectal cancer status”, “CRC status” can refer to the status of the disease in subject. Examples of types of CRC statuses include, but are not limited to, the subject's risk of cancer, including colorectal carcinoma, the presence or absence of disease (for example, polyp or adenocarcinoma), the stage of disease in a patient (for example, carcinoma), and the effectiveness of treatment of disease.

The term “mass spectrometer” can refer to a gas phase ion spectrometer that measures a parameter that can be translated into mass-to-charge (m/z) ratios of gas phase ions. Mass spectrometers generally include an ion source and a mass analyzer. Examples of mass spectrometers are time-of-flight, magnetic sector, quadrupole filter, ion trap, ion cyclotron resonance, electrostatic sector analyzer and hybrids of these. “Mass spectrometry” can refer to the use of a mass spectrometer to detect gas phase ions.

The term “tandem mass spectrometer” can refer to any mass spectrometer that is capable of performing two successive stages of m/z-based discrimination or measurement of ions, including ions in an ion mixture. The phrase includes mass spectrometers having two mass analyzers that are capable of performing two successive stages of m/z-based discrimination or measurement of ions tandem-in-space. The phrase further includes mass spectrometers having a single mass analyzer that can be capable of performing two successive stages of m/z-based discrimination or measurement of ions tandem-in-time. The phrase thus explicitly includes Qq-TOF mass spectrometers, ion trap mass spectrometers, ion trap-TOF mass spectrometers, TOF-TOF mass spectrometers, Fourier transform ion cyclotron resonance mass spectrometers, electrostatic sector-magnetic sector mass spectrometers, and combinations thereof.

The term “biochip” can refer to a solid substrate having a generally planar surface to which an adsorbent is attached. In some cases, a surface of the biochip comprises a plurality of addressable locations, each of which location may have the adsorbent bound there. Biochips can be adapted to engage a probe interface, and therefore, function as probes. Protein biochips are adapted for the capture of polypeptides and can be comprise surfaces having chromatographic or biospecific adsorbents attached thereto at addressable locations. Microarray chips are generally used for DNA and RNA gene expression detection.

The term “biomarker” and “marker” are used interchangeably herein, and can refer to a polypeptide, gene, nucleic acid (for example, DNA and/or RNA) which is differentially present in a sample taken from a subject having a disease for which a diagnosis is desired (for example, CRC) as compared to a comparable sample taken from control subject that does not have the disease (for example, a person with a negative diagnosis or undetectable CRC, normal or healthy subject, or, for example, from the same individual at a different time point). A biomarker can be a gene, such DNA or RNA or a genetic variation of the DNA or RNA, their binding partners, splice-variants. A biomarker can be a protein, protein fragment, transition ion of an amino acid sequence, or one or more modifications of a protein. In addition, a protein biomarker can be a binding partner of a protein, protein fragment, or transition ion of an amino acid sequence.

The terms “polypeptide,” “peptide” and “protein” are used interchangeably herein, and can refer to a polymer of amino acid residues. A polypeptide can be a single linear polymer chain of amino acids bonded together by peptide bonds between the carboxyl and amino groups of adjacent amino acid residues. Polypeptides can be modified, for example, by the addition of carbohydrate, phosphorylation, etc. Proteins can comprise one or more polypeptides.

An “immunoassay” can be an assay that uses an antibody to specifically bind an antigen (for example, a marker). The immunoassay can be characterized by the use of specific binding properties of a particular antibody to isolate, target, and/or quantify the antigen.

The term “antibody” can refer to a polypeptide ligand substantially encoded by an immunoglobulin gene or immunoglobulin genes, or fragments thereof, which specifically binds and recognizes an epitope. Antibodies exist, for example, as intact immunoglobulins or as a number of well-characterized fragments produced by digestion with various peptidases. This includes, for example, Fab″ and F(ab)″₂ fragments. As used herein, the term “antibody” also includes antibody fragments either produced by the modification of whole antibodies or those synthesized de novo using recombinant DNA methodologies. It also includes polyclonal antibodies, monoclonal antibodies, chimeric antibodies, humanized antibodies, or single chain antibodies. “Fc” portion of an antibody can refer to that portion of an immunoglobulin heavy chain that comprises one or more heavy chain constant region domains, but does not include the heavy chain variable region.

The term “tumor” can refer to a solid or fluid-filled lesion that may be formed by cancerous or non-cancerous cells. The terms “mass” and “nodule” are often used synonymously with “tumor”. Tumors include malignant tumors or benign tumors. An example of a malignant tumor can be a carcinoma which is known to comprise transformed cells.

The term “binding partners” can refer to pairs of molecules, typically pairs of biomolecules that exhibit specific binding. Protein—protein interactions can occur between two or more proteins, when bound together they often to carry out their biological function. Interactions between proteins are important for the majority of biological functions. For example, signals from the exterior of a cell are mediated via ligand receptor proteins to the inside of that cell by protein—protein interactions of the signaling molecules. For example, molecular binding partners include, without limitation, receptor and ligand, antibody and antigen, biotin and avidin, and others.

The term “control reference” can refer to a known or determined amount of a biomarker associated with a known condition that can be used to compare to an amount of the biomarker associated with an unknown condition. A control reference can also refer to a steady-state molecule which can be used to calibrate or normalize values of a non-steady state molecule. A control reference value can be a calculated value from a combination of factors or a combination of a range of factors, such as a combination of biomarker concentrations or a combination of ranges of concentrations.

The terms “subject,” “individual” or “patient” are used interchangeably herein. A “subject” can be a biological entity containing expressed genetic materials. The biological entity can be a plant, animal, or microorganism, including, for example, bacteria, viruses, fungi, and protozoa. The subject can be tissues, cells and their progeny of a biological entity obtained in vivo or cultured in vitro. The subject can be a mammal. The mammal can be a human. The subject may be diagnosed or suspected of being at high risk for a disease. The disease can be cancer. The cancer can be CRC (CRC). In some cases, the subject is not necessarily diagnosed or suspected of being at high risk for the disease.

The term “in vivo” can refer to an event that takes place in a subject's body.

The term “in vitro” can refer to an event that takes places outside of a subject's body. In vitro assays can encompass cell-based assays in which cells alive or dead are employed. In vitro assays can also encompass a cell-free assay in which no intact cells are employed.

The term specificity, or true negative rate, can refer to a test's ability to exclude a condition correctly. For example, in a diagnostic test, the specificity of a test is the proportion of patients known not to have the disease, who will test negative for it. In some cases, this is calculated by determining the proportion of true negatives (i.e. patients who test negative who do not have the disease) to the total number of healthy individuals in the population (i.e., the sum of patients who test negative and do not have the disease and patients who test positive and do not have the disease).

The term sensitivity, or true positive rate, can refer to a test's ability to identify a condition correctly. For example, in a diagnostic test, the sensitivity of a test is the proportion of patients known to have the disease, who will test positive for it. In some cases, this is calculated by determining the proportion of true positives (i.e. patients who test positive who have the disease) to the total number of individuals in the population with the condition (i.e., the sum of patients who test positive and have the condition and patients who test negative and have the condition).

As used herein, the term ‘about’ a number refers to that number plus or minus 10% of that number. The term ‘about’ a range refers to that range minus 10% of its lowest value and plus 10% of its greatest value.

As used herein, the terms “treatment” or “treating” are used interchangeably herein. These terms can refer to an approach for obtaining beneficial or desired results including but not limited to a therapeutic benefit and/or a prophylactic benefit. A therapeutic benefit can mean eradication or amelioration of the underlying disorder being treated. Also, a therapeutic benefit can be achieved with the eradication or amelioration of one or more of the physiological symptoms associated with the underlying disorder such that an improvement is observed in the subject, notwithstanding that the subject may still be afflicted with the underlying disorder. A prophylactic effect includes delaying, preventing, or eliminating the appearance of a disease or condition, delaying or eliminating the onset of symptoms of a disease or condition, slowing, halting, or reversing the progression of a disease or condition, or any combination thereof. For prophylactic benefit, a subject at risk of developing a particular disease, or to a subject reporting one or more of the physiological symptoms of a disease may undergo treatment, even though a diagnosis of this disease may not have been made.

### DETAILED DESCRIPTION

Provided herein are biomarker panels, methods, compositions, kits, and systems for the non-invasive detection of at least one of advanced colorectal adenoma and CRC. Any of the biomarker panels, methods, compositions, kits, and systems described herein can be used to determine a likelihood that a subject has at least one of an advanced colorectal adenoma and CRC. Such biomarker panels, methods, compositions, and kits can detect at least one of advanced colorectal adenoma and CRC with at least one of high sensitivity and high specificity. For example, the biomarker panels, methods, compositions, kits provided herein can detect at least one of advanced colorectal adenoma and CRC with a sensitivity that is at least 50%, at least 55%, at least 60%, at least 65%, at least 70%, at least 75%, at least 80%, at least 85%, at least 90%, at least 95%, at least 96%, at least 97%, at least 98%, at least 99%, or about 100%. For example, methods and kits herein can detect at least one of advanced colorectal adenoma and CRC with a sensitivity of 70%. For example, methods and kits herein can detect at least one of advanced colorectal adenoma and CRC with a sensitivity of 75%. For example, methods and kits herein can detect at least one of advanced colorectal adenoma and CRC with a sensitivity of 80%. For example, methods and kits herein can detect at least one of advanced colorectal adenoma and CRC with a sensitivity of 85%. For example, methods and kits herein can detect at least one of advanced colorectal adenoma and CRC with a sensitivity of 90%.

The biomarker panels, methods, compositions, kits provided herein can detect at least one of advanced colorectal adenoma and CRC with a specificity that is at least 50%, at least 55%, at least 60%, at least 65%, at least 70%, at least 75%, at least 80%, at least 85%, at least 90%, at least 95%, at least 96%, at least 97%, at least 98%, at least 99%, or about 100%. For example, methods and kits herein can detect at least one of advanced colorectal adenoma and CRC with a specificity of 70%. For example, methods and kits herein can detect at least one of advanced colorectal adenoma and CRC with a specificity of 75%. For example, methods and kits herein can detect at least one of advanced colorectal adenoma and CRC with a specificity of 80%. For example, methods and kits herein can detect at least one of advanced colorectal adenoma and CRC with a specificity of 85%. For example, methods and kits herein can detect at least one of advanced colorectal adenoma and CRC with a specificity of 90%.

In some cases, the biomarker panels, diagnostic methods, kits, and compositions provided herein detect at least one of advanced colorectal adenoma and CRC with a sensitivity and specificity at least 70%. In some cases, the biomarker panels, diagnostic methods, kits, and compositions provided herein detect at least one of advanced colorectal adenoma and CRC with a sensitivity and specificity at least 75%. In some cases, the diagnostic methods, kits, and compositions provided herein detect at least one of advanced colorectal adenoma and CRC with a sensitivity and specificity at least 80%. In some cases, the diagnostic methods, kits, and compositions provided herein detect at least one of advanced colorectal adenoma and CRC with a sensitivity and specificity at least 85%. In some cases, the diagnostic methods, kits, and compositions provided herein detect at least one of advanced colorectal adenoma and CRC with a sensitivity and specificity at least 90%. Furthermore, the diagnostic methods provided herein can be performed without need of an invasive colonoscopy, sigmoidoscopy, or tissue biopsy. For example, diagnostic methods provided herein can be performed via a simple blood test.

The biomarker panels, methods, compositions, and kits described herein can provide a diagnostic assay for at least one of advanced colorectal adenoma and CRC based on detection and/or measurement of one or more biomarkers in a biological sample obtained from a subject. In some embodiments, the biological sample is a blood sample. The blood sample can be a whole blood sample, a plasma sample, or a serum sample. In some cases, a diagnostic method provided herein can detect at least one of advanced colorectal adenoma and CRC. Such diagnostic method can have at least one of a sensitivity of at least 70% and specificity of at least 70%. In some cases, a diagnostic method provided herein can detect at least one of advanced colorectal adenoma and CRC. Such diagnostic methods can have at least one of a sensitivity of 70% or greater and specificity of at least 70% based on measurement of 15 or fewer biomarkers in the biological sample. In some cases, a diagnostic method provided herein can detect at least one of advanced colorectal adenoma and CRC. Such diagnostic method can have at least one of a sensitivity at least 70% and specificity at least 70% based on measurement of no more than 2 biomarkers, 3 or fewer biomarkers, 4 or fewer biomarkers, 5 or fewer biomarkers, 6 or fewer biomarkers, 7 or fewer biomarkers, 8 or fewer biomarkers, 9 or fewer biomarkers, 10 or fewer biomarkers, 11 or fewer biomarkers, no more than 12 biomarkers, 13 or fewer biomarkers, 14 or fewer biomarkers, or 15 or fewer biomarkers.

The biomarker panels, methods, compositions, and kits described herein can also be useful as a quality control metric for a colonoscopy, sigmoidoscopy, or colon tissue biopsy. For example, a positive detection of at least one of an advanced colorectal adenoma and CRC based upon a method described herein can be used to validate a result of a colonoscopy, sigmoidoscopy, or colon tissue biopsy. For example, in some cases wherein a colonoscopy, sigmoidoscopy, or colon tissue biopsy yielded a negative result, but a method described herein yielded a positive result, such method can be used to alert a caregiver to perform another colonoscopy, sigmoidoscopy, or colon tissue biopsy.

In some cases, a method provided herein comprises (a) obtaining a biological sample from a subject; (b) measuring a panel of biomarkers in the biological sample of the subject; (c) detecting a presence or absence of at least one of advanced colorectal adenoma and CRC in the subject based upon the measuring; and (d) either (i) treating the at least one of advanced colorectal adenoma CRC and in the subject based upon the detecting, or (ii) recommending to the subject a colonoscopy, sigmoidoscopy, or colorectal tissue biopsy based upon the results of the detecting. For the purposes of one or more methods described herein, “treating” comprises providing a written report to the subject or to a caretaker of the subject which includes a recommendation to initiate a treatment for the CRC. For the purposes of one or more methods described herein, “recommending to the subject a colonoscopy” comprises providing a written report to the subject or to a caretaker of the subject which includes a recommendation that the subject undergo a colonoscopy, sigmoidoscopy, or tissue biopsy to confirm a diagnosis of the CRC. In some cases, the colonoscopy, sigmoidoscopy, or tissue biopsy can be used to remove the at least one of advanced colorectal adenoma and CRC, thereby treating the at least one of advanced colorectal adenoma and CRC.

An exemplary method comprises (a) obtaining data comprising a measurement of a biomarker panel in a biological sample obtained from a subject, (b) generating a subject-specific profile of the biomarker panel based upon the measurement data, (c) comparing the subject-specific profile of the biomarker panel to a reference profile of the biomarker panel; and (d) determining a likelihood of at least one of advanced colorectal adenoma and colorectal cancer based upon (c).

An exemplary method can comprise (a) measuring a biomarker panel in a biological sample obtained from the subject; (b) detecting a presence or absence of colorectal cancer and/or advanced colorectal adenoma in the subject based upon the measuring; and (c) treating the colorectal cancer in the subject based upon the detecting.

An exemplary method can comprise (a) obtaining data comprising a measurement of a biomarker panel in a biological sample obtained from a subject, (b) generating a subject-specific profile of the biomarker panel based upon the measurement data, (c) comparing the subject-specific profile of the biomarker panel to a reference profile of the biomarker panel; and (d) determining a likelihood of at least one of advanced colorectal adenoma and colorectal cancer based upon (c). In some cases, a method provided herein comprises (a) measuring a biomarker panel in a biological sample obtained from the subject; (b) detecting a presence or absence of colorectal cancer and/or advanced colorectal adenoma in the subject based upon the measuring; and (c) recommending to the subject at least one of a colonoscopy, sigmoidoscopy, and tissue biopsy in the subject based upon the detecting.

### Algorithm-Based Methods

Any of the methods, compositions, kits, and systems described herein can utilize an algorithm-based diagnostic assay for predicting a presence or absence of at least one of: advanced colorectal adenoma and CRC in a subject. Expression level of one or more protein biomarker, and optionally one or more subject characteristics, such as, for example, age, weight, gender, medical history, risk factors, family history, and the like, may be used alone or arranged into functional subsets to calculate a quantitative score that can be used to predict the likelihood of a presence or absence of at least one of advanced colorectal adenoma and CRC.

The algorithm-based assay and associated information provided by the practice of any of the methods described herein can facilitate optimal treatment decision-making in subjects. For example, such a clinical tool can enable a physician or caretaker to identify patients who have a low likelihood of having an advanced colorectal adenoma or carcinoma and therefore would not need anti-cancer treatment, or who have a high likelihood of having an advanced colorectal adenoma or CRC and therefore would need anti-cancer treatment.

A quantitative score may be determined by the application of a specific algorithm. The algorithm used to calculate the quantitative score in the methods disclosed herein may group the expression level values of a biomarker or groups of biomarkers. The formation of a particular group of biomarkers, in addition, can facilitate the mathematical weighting of the contribution of various expression levels of biomarker or biomarker subsets (for example classifier) to the quantitative score. Described herein are exemplary algorithms for calculating the quantitative scores.

### Biomarkers

In some cases, biomarker panels described herein comprise at least two biomarkers. The biomarkers can be selected from the group consisting of A1AG1, A1AT, AACT, AMY2B, ANXA1, APOA1, CAH1, CATD, CATS, CEAM3, CLUS, CTNB1, CO3, CO9, CRP, CSF1, DPP4, ECH1, FHL1, FIBB, FIBG, FRIL, FUCO, GELS, HPT, OSTP, PRDX1, SAM, SAHH, SBP1, SEPR, SPB6, SPON2, SYG, TIMP1, and TRFE, or fragments thereof. Any of the biomarkers described herein can be protein biomarkers.

In another embodiment, the biomarker panels described herein comprise at least two biomarkers. The biomarkers can be selected from the group consisting of A1AG1, A1AT, AACT, APOA1, CATD, CEAM3, CLUS, CO3, CO9, CRP, FIBB, FIBG, GELS, OSTP, PRDX1, SAA1, SBP1, and SEPR. Any of the biomarkers described herein can be protein biomarkers.

Exemplary biomarkers, and their human amino acid sequences, are listed in Table 1, below.

TABLE 1
Biomarkers for diagnosis of CRC

| | Abbreviated | |
|---|---|---|
| Protein Name | Name | Sequence |
| Alpha-1-acid | A1AG1 | MALSWVLTVLSLLPLLEAQIPLCANLVPVPITNATLDQITGK |
| glycoprotein 1 | | WFYIASAFRNEEYNKSVQEIQATFFYFTPNKTEDTIFLREYQ |
| | | TRQDQCIYNTTYLNVQRENGTISRYVGGQEHFAHLLILRDT |
| | | KTYMLAFDVNDEKNWGLSVYADKPETTKEQLGEFYEALD |
| | | CLRIPKSDVVYTDWKKDKCEPLEKQHEKERKQEEGES |
| | | SEQ ID NO: 1 |
| Alpha-1 | A1AT | MPSSVSWGILLLAGLCCLVPVSLAEDPQGDAAQKTDTSHH |
| Antitrypsin | | DQDHPTFNKITPNLAEFAFSLYRQLAHQSNSTNIFFSPVSIAT |
| | | AFAMLSLGTKADTHDEILEGLNFNLTEIPEAQIHEGFQELLR |
| | | TLNQPDSQLQLTTGNGLFLSEGLKLVDKFLEDVKKLYHSE |
| | | AFTVNFGDTEEAKKQINDYVEKGTQGKIVDLVKELDRDTV |
| | | FALVNYIFFKGKWERPFEVKDTEEEDFHVDQVTTVKVPMM |
| | | KRLGMFNIQHCKKLSSWVLLMKYLGNATAIFFLPDEGKLQ |
| | | HLENELTHDIITKFLENEDRRSASLHLPKLSITGTYDLKSVL |
| | | GQLGITKVFSNGADLSGVTEEAPLKLSKAVHKAVLTIDEKG |
| | | TEAAGAMFLEAIPMSIPPEVKFNKPFVFLMIEQNTKSPLFMG |
| | | KVVNPTQK |
| | | SEQ ID NO: 2 |
| Alpha-1- | AACT | MERMLPLLALGLLAAGFCPAVLCHPNSPLDEENLTQENQD |
| Antichymotrypsin | | RGTHVDLGLASANVDFAFSLYKQLVLKAPDKNVIFSPLSIS |
| | | TALAFLSLGAHNTTLTEILKGLKFNLTETSEAEIHQSFQHLL |
| | | RTLNQSSDELQLSMGNAMFVKEQLSLLDRFTEDAKRLYGS |
| | | EAFATDFQDSAAAKKLINDYVKNGTRGKITDLIKDLDSQT |
| | | MMVLVNYIFFKAKWEMPFDPQDTHQSRFYLSKKKWVMVP |
| | | MMSLHHLTIPYFRDEELSCTVVELKYTGNASALFILPDQDK |
| | | MEEVEAMLLPETLKRWRDSLEFREIGELYLPKFSISRDYNL |
| | | NDILLQLGIEEAFTSKADLSGITGARNLAVSQVVHKAVLDV |
| | | FEEGTEASAATAVKITLLSALVETRTIVRFNRPFLMIIVPTDT |
| | | QNIFFMSKVTNPKQA |
| | | SEQ ID NO: 3 |
| Alpha-amylase 2B | AMY2B | MKFFLLLFTIGFCWAQYSPNTQQGRTSIVHLFEWRWVDIAL |
| | | ECERYLAPKGFGGVQVSPPNENVAIHNPFRPWWERYQPVS |
| | | YKLCTRSGNEDEFRNMVTRCNNVGVRIYVDAVINHMSGN |
| | | AVSAGTSSTCGSYFNPGSRDFPAVPYSGWDFNDGKCKTGS |
| | | GDIENYNDATQVRDCRLVGLLDLALEKDYVRSKIAEYMNH |
| | | LIDIGVAGFRLDASKHMWPGDIKAILDKLHNLNSNWFPAG |
| | | SKPFIYQEVIDLGGEPIKSSDYFGNGRVTEFKYGAKLGTVIR |
| | | KWNGEKMSYLKNWGEGWGFMPSDRALVFVDNHDNQRG |
| | | HGAGGASILTFWDARLYKMAVGFMLAHPYGFTRVMSSYR |
| | | WPRQFQNGNDVNDWVGPPNNNGVIKEVTINPDTTCGNDW |
| | | VCEHRWRQIRNMVNFRNVVDGQPFTNWYDNGSNQVAFG |
| | | RGNRGFIVFNNDDWTFSLTLQTGLPAGTYCDVISGDKINGN |
| | | CTGIKIYVSDDGKAHFSISNSAEDPFIAIHAESKL |
| | | SEQ ID NO: 4 |
| Annexin A1 | ANXA1 | MAMVSEFLKQAWFIENEEQEYVQTVKSSKGGPGSAVSPYP |
| | | TFNPSSDVAALHKAIMVKGVDEATIIDILTKRNNAQRQQIK |
| | | AAYLQETGKPLDETLKKALTGHLEEVVLALLKTPAQFDAD |
| | | ELRAAMKGLGTDEDTLIEILASRTNKEIRDINRVYREELKRD |
| | | LAKDITSDTSGDFRNALLSLAKGDRSEDFGVNEDLADSDAR |
| | | ALYEAGERRKGTDVNVFNTILTTRSYPQLRRVFQKYTKYS |
| | | KHDMNKVLDLELKGDIEKCLTAIVKCATSKPAFFAEKLHQ |
| | | AMKGVGTRHKALIRIMVSRSEIDMNDIKAFYQKMYGISLC |
| | | QAILDETKGDYEKILVALCGGN |
| | | SEQ ID NO: 5 |
| Apolipoprotein A-1 | APOA 1 | MKAAVLTLAVLFLTGSQARHFWQQDEPPQSPWDRVKDLA |
| | | TVYVDVLKDSGRDYVSQFEGSALGKQLNLKLLDNWDSVT |
| | | STFSKLREQLGPVTQEFWDNLEKETEGLRQEMSKDLEEVK |
| | | AKVQPYLDDFQKKWQEEMELYRQKVEPLRAELQEGARQK |
| | | LHELQEKLSPLGEEMRDRARAHVDALRTHLAPYSDELRQR |
| | | LAARLEALKENGGARLAEYHAKATEHLSTLSEKAKPALED |
| | | LRQGLLPVLESFKVSFLSALEEYTKKLNTQ |
| | | SEQ ID NO: 6 |
| Carbonic | CAH1 | MASPDWGYDDKNGPEQWSKLYPIANGNNQSPVDIKTSETK |
| anhydrase 1 | | HDTSLKPISVSYNPATAKEIINVGHSFHVNFEDNDNRSVLK |
| | | GGPFSDSYRLFQFHFHWGSTNEHGSEHTVDGVKYSAELHV |
| | | AHWNSAKYSSLAEAASKADGLAVIGVLMKVGEANPKLQK |
| | | VLDALQAIKTKGKRAPFTNFDPSTLLPSSLDFWTYPGSLTHP |
| | | PLYESVTWIICKESISVSSEQLAQFRSLLSNVEGDNAV |
| | | PMQHNNRPTQPLKGRTVRASF |
| | | SEQ ID NO: 7 |
| Cathepsin D | CATD | MQPSSLLPLALCLLAAPASALVRIPLHKFTSIRRTMSEVGGS |
| | | VEDLIAKGPVSKYSQAVPAVTEGPIPEVLKNYMDAQYYGEI |
| | | GIGTPPQCFTVVFDTGSSNLWVPSIHCKLLDIACWIHHKYNS |
| | | DKSSTYVKNGTSFDIHYGSGSLSGYLSQDTVSVPCQSASSA |
| | | SALGGVKVERQVFGEATKQPGITFIAAKFDGILGMAYPRIS |
| | | VNNVLPVFDNLMQQKLVDQNIFSFYLSRDPDAQPGGELML |
| | | GGTDSKYYKGSLSYLNVTRKAYWQVHLDQVEVASGLTLC |
| | | KEGCEAIVDTGTSLMVGPVDEVRELQKAIGAVPLIQGEYMI |
| | | PCEKVSTLPAITLKLGGKGYKLSPEDYTLKVSQAGKTLCLS |
| | | GFMGMDIPPPSGPLWILGDVFIGRYYTVFDRDNNRVGFAEA |
| | | ARL |
| | | SEQ ID NO: 8 |
| Cathepsin S | CATS | MKRLVCVLLVCSSAVAQLHKDPTLDHHWHLWKKTYGKQ |
| | | YKEKNEEAVRRLIWEKNLKFVMLHNLEHSMGMHSYDLG |
| | | MNHLGDMTSEEVMSLMSSLRVPSQWQRNITYKSNPNRILP |
| | | DSVDWREKGCVTEVKYQGSCGACWAFSAVGALEAQLKL |
| | | KTGKLVSLSAQNLVDCSTEKYGNKGCNGGFMTTAFQYIID |
| | | NKGIDSDASYPYKAMDQKCQYDSKYRAATCSKYTELPYG |
| | | REDVLKEAVANKGPVSVGVDARHPSFFLYRSGVYYEPSCT |
| | | QNVNHGVLVVGYGDLNGKEYWLVKNSWGHNFGEEGYIR |
| | | MARNKGNHCGIASFPSYPEI |
| | | SEQ ID NO: 9 |
| Carcinoembryonic | CEAM3 | MGPPSASPHRECIPWQGLLLTASLLNFWNPPTTAKLTIESMP |
| antigen-related cell | | LSVAEGKEVLLLVHNLPQHLFGYSWYKGERVDGNSLIVGY |
| adhesion molecule | | VIGTQQATPGAAYSGRETIYTNASLLIQNVTQNDIGFYTLQ |
| 3 | | VIKSDLVNEEATGQFHVYQENAPGLPVGAVAGIVTGVLVG |
| | | VALVAALVCFLLLAKTGRTSIQRDLKEQQPQALAPGRGPSH |
| | | SSAFSMSPLSTAQAPLPNPRTAASIYEELLKHDTNIYCRMDH |
| | | KAEVAS |
| | | SEQ ID NO: 10 |
| Clusterin | CLUS | MMKTLLLFVGLLLTWESGQVLGDQTVSDNELQEMSNQGS |
| | | KYVNKEIQNAVNGVKQIKTLIEKTNEERKTLLSNLEEAKKK |
| | | KEDALNETRESETKLKELPGVCNETMMALWEECKPCLKQT |
| | | CMKFYARVCRSGSGLVGRQLEEFLNQSSPFYFWMNGDRID |
| | | SLLENDRQQTHMLDVMQDHFSRASSIIDELFQDRFFTREPQ |
| | | DTYHYLPFSLPHRRPHFFFPKSRIVRSLMPFSPYEPLNFHAM |
| | | FQPFLEMIHEAQQAMDIHFHSPAFQHPPTEFIREGDDDRTVC |
| | | REIRHNSTGCLRMKDQCDKCREILSVDCSTNNPSQAKLRRE |
| | | LDESLQVAERLTRKYNELLKSYQWKMLNTSSLLEQLNEQF |
| | | NWVSRLANLTQGEDQYYLRVTTVASHTSDSDVPSGVTEVV |
| | | VKLFDSDPITVTVPVEVSRKNPKFMETVAEKALQEYRKKH |
| | | REE |
| | | SEQ ID NO: 11 |
| Catenin beta-1 | CTNB1 | MATQADLMELDMAMEPDRKAAVSHWQQQSYLDSGIHSG |
| | | ATTTAPSLSGKGNPEEEDVDTSQVLYEWEQGFSQSFTQEQV |
| | | ADIDGQYAMTRAQRVRAAMFPETLDEGMQIPSTQFDAAHP |
| | | TNVQRLAEPSQMLKHAVVNLINYQDDAELATRAIPELTKL |
| | | LNDEDQVVVNKAAVMVHQLSKKEASRHAIMRSPQMVSAI |
| | | VRTMQNTNDVETARCTAGTLHNLSHHREGLLAIFKSGGIPA |
| | | LVKMLGSPVDSVLFYAITTLHNLLLHQEGAKMAVRLAGGL |
| | | QKMVALLNKTNVKFLAITTDCLQILAYGNQESKLIILASGG |
| | | PQALVNIMRTYTYEKLLWTTSRVLKVLSVCSSNKPAIVEA |
| | | GGMQALGLHLTDPSQRLVQNCLWTLRNLSDAATKQEGME |
| | | GLLGTLVQLLGSDDINVVTCAAGILSNLTCNNYKNKMMVC |
| | | QVGGIEALVRTVLRAGDREDITEPAICALRHLTSRHQEAEM |
| | | AQNAVRLHYGLPVVVKLLHPPSHWPLIKATVGLIRNLALCP |
| | | ANHAPLREQGAIPRLVQLLVRAHQDTQRRTSMGGTQQQFV |
| | | EGVRMEEIVEGCTGALHILARDVHNRIVIRGLNTIPLFV |
| | | QLLYSPIENIQRVAAGVLCELAQDKEAAEAIEAEGATAPLT |
| | | ELLHSRNEGVATYAAAVLFRMSEDKPQDYKKRLSVELTSS |
| | | LFRTEPMAWNETADLGLDIGAQGEPLGYRQDDPSYRSFH |
| | | SGGYGQDALGMDPMMEHEMGGHHPGADYPVDGLPDLGH |
| | | AQDLMDGLPPGDSNQLAWFDTDL |
| | | SEQ ID NO: 12 |
| Complement C3 | CO3 | MGPTSGPSLLLLLLTHLPLALGSPMYSIITPNILRLESEETMV |
| | | LEAHDAQGDVPVTVTVHDFPGKKLVLSSEKTVLTPATNHM |
| | | GNVTFTIPANREFKSEKGRNKFVTVQATFGTQVVEKVVLV |
| | | SLQSGYLFIQTDKTIYTPGSTVLYRIFTVNHKLLPVGRTVMV |
| | | NIENPEGIPVKQDSLSSQNQLGVLPLSWDIPELVNMGQWKI |
| | | RAYYENSPQQVFSTEFEVKEYVLPSFEVIVEPTEKFYYIYNE |
| | | KGLEVTITARFLYGKKVEGTAFVIFGIQDGEQRISLPESLKRI |
| | | PIEDGSGEVVLSRKVLLDGVQNPRAEDLVGKSLYVSATVIL |
| | | HSGSDMVQAERSGIPIVTSPYQIHFTKTPKYFKPGMPFDLM |
| | | VFVTNPDGSPAYRVPVAVQGEDTVQSLTQGDGVAKLSINT |
| | | HPSQKPLSITVRTKKQELSEAEQATRTMQALPYSTVGNSNN |
| | | YLHLSVLRTELRPGETLNVNFLLRMDRAHEAKIRYYTYLIM |
| | | NKGRLLKAGRQVREPGQDLVVLPLSITTDFIPSFRLVAYYT |
| | | LIGASGQREVVADSVWVDVKDSCVGSLVVKSGQSEDRQP |
| | | VPGQQMTLKIEGDHGARVVLVAVDKGVFVLNKKNKLTQS |
| | | KIWDVVEKADIGCTPGSGKDYAGVFSDAGLTFTSSSGQQT |
| | | AQRAELQCPQPAARRRRSVQLTEKRMDKVGKYPKELRKC |
| | | CEDGMRENPMRFSCQRRTRFISLGEACKKVFLDCCNYITEL |
| | | RRQHARASHLGLARSNLDEDIIAEENIVSRSEEPESWLWNV |
| | | EDLKEPPKNGISTKLMNIFLKDSITTWEILAVSMSDKKGICV |
| | | ADPFEVTVMQDFFIDLRLPYSVVRNEQVEIRAVLYNYRQN |
| | | QELKVRVELLHNPAFCSLATTKRRHQQTVTIPPKSSLSVPY |
| | | VIVPLKTGLQEVEVKAAVYHHFISDGVRKSLKVVPEGIRMN |
| | | KTVAVRTLDPERLGREGVQKEDIPPADLSDQVPDTESETRIL |
| | | LQGTPVAQMTEDAVDAERLKHLIVTPSGCGEQNMIGMTPT |
| | | VIAVHYLDETEQWEKFGLEKRQGALELIKKGYTQQLAFRQ |
| | | PSSAFAAFVKRAPSTWLTAYVVKVFSLAVNLIAIDSQVLCG |
| | | AVKWLILEKQKPDGVFQEDAPVIHQEMIGGLRNNNEKDM |
| | | ALTAFVLISLQEAKDICEEQVNSLPGSITKAGDFLEANYMN |
| | | LQRSYTVAIAGYALAQMGRLKGPLLNKFLTTAKDKNRWE |
| | | DPGKQLYNVEATSYALLALLQLKDFDFVPPVVRWLNEQRY |
| | | YGGGYGSTQATFMVFQALAQYQKDAPDHQELNLDVSLQL |
| | | PSRSSKITHRIHWESASLLRSEETKENEGFTVTAEGKGQGTL |
| | | SVVTMYHAKAKDQLTCNKFDLKVTIKPAPETEKRPQDAKN |
| | | TMILEICTRYRGDQDATMSILDISMMTGFAPDTDDLKQLAN |
| | | GVDRYISKYELDKAFSDRNTLIIYLDKVSHSEDDCLAFKVH |
| | | QYFNVELIQPGAVKVYAYYNLEESCTRFYHPEKEDGKLNK |
| | | LCRDELCRCAEENCFIQKSDDKVTLEERLDKACEPGVDYV |
| | | YKTRLVKVQLSNDFDEYIMAIEQTIKSGSDEVQVGQQRTFI |
| | | SPIKCREALKLEEKKHYLMWGLSSDFWGEKPNLSYIIGKDT |
| | | WVEHWPEEDECQDEENQKQCQDLGAFTESMVVFGCPN |
| | | SEQ ID NO: 13 |
| Complement C9 | CO9 | MSACRSFAVAICILEISILTAQYTTSYDPELTESSGSASHIDC |
| | | RMSPWSEWSQCDPCLRQMFRSRSIEVFGQFNGKRCTDAVG |
| | | DRRQCVPTEPCEDAEDDCGNDFQCSTGRCIKMRLRCNGDN |
| | | DCGDFSDEDDCESEPRPPCRDRVVEESELARTAGYGINILG |
| | | MDPLSTPFDNEFYNGLCNRDRDGNTLTYYRRPWNVASLIY |
| | | ETKGEKNFRTEHYEEQIEAFKSIIQEKTSNFNAAISLKFTPTE |
| | | TNKAEQCCEETASSISLHGKGSFRFSYSKNETYQLFLSYSSK |
| | | KEKMFLHVKGEIHLGRFVMRNRDVVLTTTFVDDIKALPTT |
| | | YEKGEYFAFLETYGTHYSSSGSLGGLYELIYVLDKASMKR |
| | | KGVELKDIKRCLGYHLDVSLAFSEISVGAEFNKDDCVKRGE |
| | | GRAVNITSENLIDDVVSLIRGGTRKYAFELKEKLLRGTVIDV |
| | | TDFVNWASSINDAPVLISQKLSPIYNLVPVKMKNAHLKKQ |
| | | NLERAIEDYINEFSVRKCHTCQNGGTVILMDGKCLCACPFK |
| | | FEGIACEISKQKISEGLPALEFPNEK |
| | | SEQ ID NO: 14 |
| C-reactive protein | CRP | MEKLLCFLVLTSLSHAFGQTDMSRKAFVFPKESDTSYVSLK |
| | | APLTKPLKAFTVCLHFYTELSSTRGYSIFSYATKRQDNEILIF |
| | | WSKDIGYSFTVGGSEILFEVPEVTVAPVHICTSWESASGIVE |
| | | FWVDGKPRVRKSLKKGYTVGAEASIILGQEQDSFGGNFEG |
| | | SQSLVGDIGNVNMWDFVLSPDEINTIYLGGPFSPNVLNWRA |
| | | LKYEVQGEVFTKPQLWP |
| | | SEQ ID NO: 15 |
| Macrophage | CSF1 | MTAPGAAGRCPPTTWLGSLLLLVCLLASRSITEEVSEYCSH |
| colony-stimulating | | MIGSGHLQSLQRLIDSQMETSCQITFEFVDQEQLKDPVCYL |
| factor 1 | | KKAFLLVQDIMEDTMRFRDNTPNAIAIVQLQELSLRLK |
| | | SCFTKDYEEHDKACVRTFYETPLQLLEKVKNVFNETKNLL |
| | | DKDWNIFSKNCNNSFAECSSQDVVTKPDCNCLYPKAIPSSD |
| | | PASVSPHQPLAPSMAPVAGLTWEDSEGTEGSSLLPGEQP |
| | | LHTVDPGSAKQRPPRSTCQSFEPPETPVVKDSTIGGSPQPRP |
| | | SVGAFNPGMEDILDSAMGTNWVPEEASGEASEIPVPQGTEL |
| | | SPSRPGGGSMQTEPARPSNFLSASSPLPASAKGQQPA |
| | | DVTGTALPRVGPVRPTGQDWNHTPQKTDHPSALLRDPPEP |
| | | GSPRISSLRPQGLSNPSTLSAQPQLSRSHSSGSVLPLGELEGR |
| | | RSTRDRRSPAEPEGGPASEGAARPLPRFNSVPLTDTGHERQ |
| | | SEGSFSPQLQESVFHLLVPSVILVLLAVGGLLFYRWRRRSH |
| | | QEPQRADSPLEQPEGSPLTQDDRQVELPV |
| | | SEQ ID NO: 16 |
| Dipeptidyl | DPP4 | MKTPWKVLLGLLGAAALVTIITVPVVLLNKGTDDATADSR |
| peptidase 4 | | KTYTLTDYLKNTYRLKLYSLRWISDHEYLYKQENNILVFN |
| | | AEYGNSSVFLENSTFDEFGHSINDYSISPDGQFILLEYNY |
| | | VKQWRHSYTASYDIYDLNKRQLITEERIPNNTQWVTWSPV |
| | | GHKLAYVWNNDIYVKIEPNLPSYRITWTGKEDIIYNGITDW |
| | | VYEEEVFSAYSALWWSPNGTFLAYAQFNDTEVPLIEYSF |
| | | YSDESLQYPKTVRVPYPKAGAVNPTVKFFVVNTDSLSSVT |
| | | NATSIQITAPASMLIGDHYLCDVTWATQERISLQWLRRIQN |
| | | YSVMDICDYDESSGRWNCLVARQHIEMSTTGWVGRFRPS |
| | | EPHFTLDGNSFYKIISNEEGYRHICYFQIDKKDCTFITKGTW |
| | | EVIGIEALTSDYLYYISNEYKGMPGGRNLYKIQLSDYTKVT |
| | | CLSCELNPERCQYYSVSFSKEAKYYQLRCSGPGLPLY |
| | | TLHSSVNDKGLRVLEDNSALDKMLQNVQMPSKKLDFIILN |
| | | ETKFWYQMILPPHFDKSKKYPLLLDVYAGPCSQKADTVFR |
| | | LNWATYLASTENIIVASFDGRGSGYQGDKIMHAINRRLGT |
| | | FEVEDQIEAARQFSKMGFVDNKRIAIWGWSYGGYVTSMVL |
| | | GSGSGVFKCGIAVAPVSRWEYYDSVYTERYMGLPTPEDNL |
| | | DHYRNSTVMSRAENFKQVEYLLIHGTADDNVHFQQSAQIS |
| | | KALVDVGVDFQAMWYTDEDHGIASSTAHQHIYTHMSHFIK |
| | | QCFSLP |
| | | SEQ ID NO: 17 |
| Delta(3,5)- | ECH1 | MAAGIVASRRLRDLLTRRLTGSNYPGLSISLRLTGSSAQEEA |
| Delta(2,4)-dienoyl- | | SGVALGEAPDHSYESLRVTSAQKHVLHVQLNRPNKRNAM |
| CoA isomerase, | | NKVFWREMVECFNKISRDADCRAVVISGAGKMFTAGIDL |
| mitochondrial | | MDMASDILQPKGDDVARISWYLRDIITRYQETFNVIERCPK |
| | | PVIAAVHGGCIGGGVDLVTACDIRYCAQDAFFQVKEVDVG |
| | | LAADVGTLQRLPKVIGNQSLVNELAFTARKMMADEALGS |
| | | GLVSRVFPDKEVMLDAALALAAEISSKSPVAVQSTKVNLL |
| | | YSRDHSVAESLNYVASWNMSMLQTQDLVKSVQATTENKE |
| | | LKTVTFSKL |
| | | SEQ ID NO: 18 |
| Four and a half | FHL1 | MAEKFDCHYCRDPLQGKKYVQKDGHHCCLKCFDKFCANT |
| LIM domains | | CVECRKPIGADSKEVHYKNRFWHDTCFRCAKCLHPLANET |
| protein 1 | | FVAKDNKILCNKCTTREDSPKCKGCFKAIVAGDQNVEYKG |
| | | TVWHKDCFTCSNCKQVIGTGSFFPKGEDFYCVTCHETKFA |
| | | KHCVKCNKAITSGGITYQDQPWHADCFVCVTCSKKLAGQR |
| | | FTAVEDQYYCVDCYKNFVAKKCAGCKNPITGKRTVSRVSH |
| | | PVSKARKPPVCHGKRLPLTLFPSANLRGRHPGGERTCPSWV |
| | | VVLYRKNRSLAAPRGPGLVKAPVWWPMKDNPGTTTASTA |
| | | KNAP |
| | | SEQ ID NO: 19 |
| Fibrinogen beta | FIBB | MKRMVSWSFHKLKTMKHLLLLLLCVFLVKSQGVNDNEEG |
| chain | | FFSARGHRPLDKKREEAPSLRPAPPPISGGGYRARPAKAAA |
| | | TQKKVERKAPDAGGCLHADPDLGVLCPTGCQLQEALLQQ |
| | | ERPIRNSVDELNNNVEAVSQTSSSSFQYMYLLKDLWQKRQ |
| | | KQVKDNENVVNEYSSELEKHQLYIDETVNSNIPTNLRVLRS |
| | | ILENLRSKIQKLESDVSAQMEYCRTPCTVSCNIPVVSGKECE |
| | | EIIRKGGETSEMYLIQPDSSVKPYRVYCDMNTENGGWTVIQ |
| | | NRQDGSVDFGRKWDPYKQGFGNVATNTDGKNYCGLPGE |
| | | YWLGNDKISQLTRMGPTELLIEMEDWKGDKVKAHYGGFT |
| | | VQNEANKYQISVNKYRGTAGNALMDGASQLMGENRTMTI |
| | | HNGMFFSTYDRDNDGWLTSDPRKQCSKEDGGGWWYNRC |
| | | HAANPNGRYYWGGQYTWDMAKHGTDDGVVWMNWKGS |
| | | WYSMRKMSMKIRPFFPQQ |
| | | SEQ ID NO: 20 |
| Fibrinogen gamma | FIBG | MSWSLHPRNLILYFYALLFLSSTCVAYVATRDNCCILDERF |
| chain | | GSYCPTTCGIADFLSTYQTKVDKDLQSLEDILHQVENKTSE |
| | | VKQLIKAIQLTYNPDESSKPNMIDAATLKSRKMLEEIMKYE |
| | | ASILTHDSSIRYLQEIYNSNNQKIVNLKEKVAQLEAQCQEPC |
| | | KDTVQIHDITGKDCQDIANKGAKQSGLYFIKPLKANQQFLV |
| | | YCEIDGSGNGWTVFQKRLDGSVDFKKNWIQYKEGFGHLSP |
| | | TGTTEFWLGNEKIHLISTQSAIPYALRVELEDWNGRTSTAD |
| | | YAMFKVGPEADKYRLTYAYFAGGDAGDAFDGFDFGDDPS |
| | | DKFFTSHNGMQFSTWDNDNDKFEGNCAEQDGSGWWMNK |
| | | CHAGHLNGVYYQGGTYSKASTPNGYDNGIIWATWKTRWY |
| | | SMKKTTMKIIPFNRLTIGEGQQHHLGGAKQVRPEHPAETEY |
| | | DSLYPEDDL |
| | | SEQ ID NO: 21 |
| Ferritin light chain | FRIL | MSSQIRQNYSTDVEAAVNSLVNLYLQASYTYLSLGFYFDR |
| | | DDVALEGVSHFFRELAEEKREGYERLLKMQNQRGGRALFQ |
| | | DIKKPAEDEWGKTPDAMKAAMALEKKLNQALLDLHALGS |
| | | ARTDPHLCDFLETHFLDEEVKLIKKMGDHLTNLHRLGGPE |
| | | AGLGEYLFERLTLKHD |
| | | SEQ ID NO: 22 |
| Alpha-L- | FUCO | MRAPGMRSRPAGPALLLLLLFLGAAESVRRAQPPRRYTPD |
| fucosidase | | WPSLDSRPLPAWFDEAKFGVFIHWGVFSVPAWGSEWFWW |
| | | HWQGEGRPQYQRFMRDNYPPGFSYADFGPQFTARFFHPEE |
| | | WADLFQAAGAKYVVLTTKHHEGFTNWPSPVSWNWNSKD |
| | | VGPHRDLVGELGTALRKRNIRYGLYHSLLEWFHPLYLLDK |
| | | KNGFKTQHFVSAKTMPELYDLVNSYKPDLIWSDGEWECPD |
| | | TYWNSTNFLSWLYNDSPVKDEVVVNDRWGQNCSCHHGG |
| | | YYNCEDKFKPQSLPDHKWEMCTSIDKFSWGYRRDMALSD |
| | | VTEESEIISELVQTVSLGGNYLLNIGPTKDGLIVPIFQERLLA |
| | | VGKWLSINGEAIYASKPWRVQWEKNTTSVWYTSKGSAVY |
| | | AIFLHWPENGVLNLESPITTSTTKITMLGIQGDLKWSTDPDK |
| | | GLFISLPQLPPSAVPAEFAWTIKLTGVK |
| | | SEQ ID NO: 23 |
| Gelsolin | GELS | MAPHRPAPALLCALSLALCALSLPVRAATASRGASQAGAP |
| | | QGRVPEARPNSMVVEHPEFLKAGKEPGLQIWRVEKFDLVP |
| | | VPTNLYGDFFTGDAYVILKTVQLRNGNLQYDLHYWLGNE |
| | | CSQDESGAAAIFTVQLDDYLNGRAVQHREVQGFESATFLG |
| | | YFKSGLKYKKGGVASGFKHVVPNEVVVQRLFQVKGRRVV |
| | | RATEVPVSWESFNNGDCFILDLGNNIHQWCGSNSNRYERL |
| | | KATQVSKGIRDNERSGRARVHVSEEGTEPEAMLQVLGPKP |
| | | ALPAGTEDTAKEDAANRKLAKLYKVSNGAGTMSVSLVAD |
| | | ENPFAQGALKSEDCFILDHGKDGKIFVWKGKQANTEERKA |
| | | ALKTASDFITKMDYPKQTQVSVLPEGGETPLFKQFFKNWR |
| | | DPDQTDGLGLSYLSSHIANVERVPFDAATLHTSTAMAAQH |
| | | GMDDDGTGQKQIWRIEGSNKVPVDPATYGQFYGGDSYIIL |
| | | YNYRHGGRQGQIIYNWQGAQSTQDEVAASAILTAQLDEEL |
| | | GGTPVQSRVVQGKEPAHLMSLFGGKPMIIYKGGTSREGGQ |
| | | TAPASTRLFQVRANSAGATRAVEVLPKAGALNSNDAFVLK |
| | | TPSAAYLWVGTGASEAEKTGAQELLRVLRAQPVQVAEGSE |
| | | PDGFWEALGGKAAYRTSPRLKDKKMDAHPPRLFACSNKIG |
| | | RFVIEEVPGELMQEDLATDDVMLLDTWDQVFVWVGKDSQ |
| | | EEEKTEALTSAKRYIETDPANRDRRTPITVVKQGFEPPSFVG |
| | | WFLGWDDDYWSVDPLDRAMAELAA |
| | | SEQ ID NO: 24 |
| Haptoglobin | HPT | MSALGAVIALLLWGQLFAVDSGNDVTDIADDGCPKPPEIA |
| | | HGYVEHSVRYQCKNYYKLRTEGDGVYTLNDKKQWINKA |
| | | VGDKLPECEADDGCPKPPEIAHGYVEHSVRYQCKNYYKLR |
| | | TEGDGVYTLNNEKQWINKAVGDKLPECEAVCGKPKNPAN |
| | | PVQRILGGHLDAKGSFPWQAKMVSHHNLTTGATLINEQWL |
| | | LTTAKNLFLNHSENATAKDIAPTLTLYVGKKQLVEIEKVVL |
| | | HPNYSQVDIGLIKLKQKVSVNERVMPICLPSKDYAEVGRVG |
| | | YVSGWGRNANFKFTDHLKYVMLPVADQDQCIRHYEGSTV |
| | | PEKKTPKSPVGVQPILNEHTFCAGMSKYQEDTCYGDAGSA |
| | | FAVHDLEEDTWYATGILSFDKSCAVAEYGVYVKVTSIQDW |
| | | VQKTIAEN |
| | | SEQ ID NO: 25 |
| Osteopontin | OSTP | MRIAVICFCLLGITCAIPVKQADSGSSEEKQLYNKYPDAVA |
| | | TWLNPDPSQKQNLLAPQNAVSSEETNDFKQETLPSKSNESH |
| | | DHMDDMDDEDDDDHVDSQDSIDSNDSDDVDDTDDSHQSD |
| | | ESHHSDESDELVTDFPTDLPATEVFTPVVPTVDTYDGRGDS |
| | | VVYGLRSKSKKFRRPDIQYPDATDEDITSHMESEELNGAYK |
| | | AIPVAQDLNAPSDWDSRGKDSYETSQLDDQSAETHSHKQS |
| | | RLYKRKANDESNEHSDVIDSQELSKVSREFHSHEFHSHEDM |
| | | LVVDPKSKEEDKHLKFRISHELDSASSEVN |
| | | SEQ ID NO: 26 |
| Peroxiredoxin-1 | PRDX1 | MSSGNAKIGHPAPNFKATAVMPDGQFKDISLSDYKGKYVV |
| | | FFFYPLDFTFVCPTEIIAFSDRAEEFKKLNCQVIGASVDSHFC |
| | | HLAWVNTPKKQGGLGPMNIPLVSDPKRTIAQDYGVLKADE |
| | | GISFRGLFIIDDKGILRQITVNDLPVGRSVDETLRLVQAFQFT |
| | | DKHGEVCPAGWKPGSDTIKPDVQKSKEYFSKQK |
| | | SEQ ID NO: 27 |
| Serum amyloid A- | SAM | MKLLTGLVFCSLVLGVSSRSFFSFLGEAFDGARDMWRAYS |
| 1 protein | | DMREANYIGSDKYFHARGNYDAAKRGPGGVWAAEAISDA |
| | | RENIQRFFGHGAEDSLADQAANEWGRSGKDPNHFRPAGLP |
| | | EKY |
| | | SEQ ID NO: 28 |
| Adenosyl- | SAHH | MSDKLPYKVADIGLAAWGRKALDIAENEMPGLMRMRERY |
| homocysteinase | | SASKPLKGARIAGCLHMTVETAVLIETLVTLGAEVQWSSCN |
| | | IFSTQDHAAAAIAKAGIPVYAWKGETDEEYLWCIEQTLYFK |
| | | DGPLNMILDDGGDLTNLIHTKYPQLLPGIRGISEETTTGVHN |
| | | LYKMMANGILKVPAINVNDSVTKSKFDNLYGCRESLIDGIK |
| | | RATDVMIAGKVAVVAGYGDVGKGCAQALRGFGARVIITEI |
| | | DPINALQAAMEGYEVTTMDEACQEGNIFVTTTGCIDIILGR |
| | | HFEQMKDDAIVCNIGHFDVEIDVKWLNENAVEKVNIKPQV |
| | | DRYRLKNGRRIILLAEGRLVNLGCAMGHPSFVMSNSFTNQ |
| | | VMAQIELWTHPDKYPVGVHFLPKKLDEAVAEAHLGKLNV |
| | | KLTKLTEKQAQYLGMSCDGPFKPDHYRY |
| | | SEQ ID NO: 29 |
| Selenium-binding | SBP1 | MATKCGNCGPGYSTPLEAMKGPREEIVYLPCIYRNTGTEAP |
| protein 1 | | DYLATVDVDPKSPQYCQVIHRLPMPNLKDELHHSGWNTCS |
| | | SCFGDSTKSRTKLVLPSLISSRIYVVDVGSEPRAPKLHKVIEP |
| | | KDIHAKCELAFLHTSHCLASGEVMISSLGDVKGNGKGGFV |
| | | LLDGETFEVKGTWERPGGAAPLGYDFWYQPRHNVMISTE |
| | | WAAPNVLRDGFNPADVEAGLYGSHLYVWDWQRHEIVQTL |
| | | SLKDGLIPLEIRFLHNPDAAQGFVGCALSSTIQRFYKNEGGT |
| | | WSVEKVIQVPPKKVKGWLLPEMPGLITDILLSLDDRFLYFS |
| | | NWLHGDLRQYDISDPQRPRLTGQLFLGGSIVKGGPVQVLE |
| | | DEELKSQPEPLVVKGKRVAGGPQMIQLSLDGKRLYITTSLY |
| | | SAWDKQFYPDLIREGSVMLQVDVDTVKGGLKLNPNFLVDF |
| | | GKEPLGPALAHELRYPGGDCSSDIWI |
| | | SEQ ID NO: 30 |
| Seprase | SEPR | MKTWVKIVFGVATSAVLALLVMCIVLRPSRVHNSEENTMR |
| | | ALTLKDILNGTFSYKTFFPNWISGQEYLHQSADNNIVLYNIE |
| | | TGQSYTILSNRTMKSVNASNYGLSPDRQFVYLESDYSKLW |
| | | RYSYTATYYIYDLSNGEFVRGNELPRPIQYLCWSPVGSKLA |
| | | YVYQNNIYLKQRPGDPPFQITFNGRENKIFNGIPDWVYEEE |
| | | MLATKYALWWSPNGKFLAYAEFNDTDIPVIAYSYYGDEQ |
| | | YPRTINIPYPKAGAKNPVVRIFIIDTTYPAYVGPQEVPVPAMI |
| | | ASSDYYFSWLTWVTDERVCLQWLKRVQNVSVLSICDFRED |
| | | WQTWDCPKTQEHIEESRTGWAGGFFVSTPVFSYDAISYYKI |
| | | FSDKDGYKHIHYIKDTVENAIQITSGKWEAINIFRVTQDSLF |
| | | YSSNEFEEYPGRRNIYRISIGSYPPSKKCVTCHLRKERCQYY |
| | | TASFSDYAKYYALVCYGPGIPISTLHDGRTDQEIKILEENKE |
| | | LENALKNIQLPKEEIKKLEVDEITLWYKMILPPQFDRSKKYP |
| | | LLIQVYGGPCSQSVRSVFAVNWISYLASKEGMVIALVDGR |
| | | GTAFQGDKLLYAVYRKLGVYEVEDQITAVRKFIEMGFIDE |
| | | KRIAIWGWSYGGYVSSLALASGTGLFKCGIAVAPVSSWEY |
| | | YASVYTERFMGLPTKDDNLEHYKNSTVMARAEYFRNVDY |
| | | LLIHGTADDNVHFQNSAQIAKALVNAQVDFQAMWYSDQN |
| | | HGLSGLSTNHLYTHMTHFLKQCFSLSD |
| | | SEQ ID NO: 31 |
| Serpin B6 | SPB6 | MDVLAEANGTFALNLLKTLGKDNSKNVFFSPMSMSCALA |
| | | MVYMGAKGNTAAQMAQILSFNKSGGGGDIHQGFQSLLTE |
| | | VNKTGTQYLLRMANRLFGEKSCDFLSSFRDSCQKFYQAEM |
| | | EELDFISAVEKSRKHINTWVAEKTEGKIAELLSPGSVDPLTR |
| | | LVLVNAVYFRGNWDEQFDKENTEERLFKVSKNEEKPVQM |
| | | MFKQSTFKKTYIGEIFTQILVLPYVGKELNMIIMLPDETTDL |
| | | RTVEKELTYEKFVEWTRLDMMDEEEVEVSLPRFKLEESYD |
| | | MESVLRNLGMTDAFELGKADFSGMSQTDLSLSKVVHKSFV |
| | | EVNEEGTEAAAATAAIMMMRCARFVPRFCADHPFLFFIQ |
| | | HSKTNGILFCGRFSSP |
| | | SEQ ID NO: 32 |
| Spondin-2 | SPON2 | MENPSPAAALGKALCALLLATLGAAGQPLGGESICSARAL |
| | | AKYSITFTGKWSQTAFPKQYPLFRPPAQWSSLLGAAHSSDY |
| | | SMWRKNQYVSNGLRDFAERGEAWALMKEIEAAGEALQSV |
| | | HEVFSAPAVPSGTGQTSAELEVQRRHSLVSFVVRIVPSPDW |
| | | FVGVDSLDLCDGDRWREQAALDLYPYDAGTDSGFTFSSPN |
| | | FATIPQDTVTEITSSSPSHPANSFYYPRLKALPPIARVTLVRL |
| | | RQSPRAFIPPAPVLPSRDNEIVDSASVPETPLDCEVSLWSSW |
| | | GLCGGHCGRLGTKSRTRYVRVQPANNGSPCPELEEEAECV |
| | | PDNCV |
| | | SEQ ID NO: 33 |
| Tyrosine-protein | SRC | MGSNKSKPKDASQRRRSLEPAENVHGAGGGAFPASQTPSK |
| kinase Src | | PASADGHRGPSAAFAPAAAEPKLFGGFNSSDTVTSPQRAGP |
| | | LAGGVTTFVALYDYESRTETDLSFKKGERLQIVNNTEGDW |
| | | WLAHSLSTGQTGYIPSNYVAPSDSIQAEEWYFGKITRRESE |
| | | RLLLNAENPRGTFLVRESETTKGAYCLSVSDFDNAKGLNV |
| | | KHYKIRKLDSGGFYITSRTQFNSLQQLVAYYSKHADGLCH |
| | | RLTTVCPTSKPQTQGLAKDAWEIPRESLRLEVKLGQGCFGE |
| | | VWMGTWNGTTRVAIKTLKPGTMSPEAFLQEAQVMKKLRH |
| | | EKLVQLYAVVSEEPIYIVTEYMSKGSLLDFLKGETGKYLRL |
| | | PQLVDMAAQIASGMAYVERMNYVHRDLRAANILVGENLV |
| | | CKVADFGLARLIEDNEYTARQGAKFPIKWTAPEAALYGRF |
| | | TIKSDVWSFGILLTELTTKGRVPYPGMVNREVLDQVERGY |
| | | RMPCPPECPESLHDLMCQCWRKEPEERPTFEYLQAFLEDYF |
| | | TSTEPQYQPGENL |
| | | SEQ ID NO: 34 |
| Glycine--tRNA | SYG | MPSPRPVLLRGARAALLLLLPPRLLARPSLLLRRSLSAASCP |
| ligase | | PISLPAAASRSSMDGAGAEEVLAPLRLAVRQQGDLVRKLK |
| | | EDKAPQVDVDKAVAELKARKRVLEAKELALQPKDDIVDR |
| | | AKMEDTLKRRFFYDQAFAIYGGVSGLYDFGPVGCALKNNII |
| | | QTWRQHFIQEEQILEIDCTMLTPEPVLKTSGHVDKFADFMV |
| | | KDVKNGECFRADHLLKAHLQKLMSDKKCSVEKKSEMESV |
| | | LAQLDNYGQQELADLFVNYNVKSPITGNDLSPPVSFNLMF |
| | | KTFIGPGGNMPGYLRPETAQGIFLNFKRLLEFNQGKLPFAA |
| | | AQIGNSFRNEISPRSGLIRVREFTMAEIEHFVDPSEKDHPKFQ |
| | | NVADLHLYLYSAKAQVSGQSARKMRLGDAVEQGVINNTV |
| | | LGYFIGRIYLYLTKVGISPDKLRFRQHMENEMAHYACDCW |
| | | DAESKTSYGWIEIVGCADRSCYDLSCHARATKVPLVAEKPL |
| | | KEPKTVNVVQFEPSKGAIGKAYKKDAKLVMEYLAICDECY |
| | | ITEMEMLLNEKGEFTIETEGKTFQLTKDMINVKRFQKTLYV |
| | | EEVVPNVIEPSFGLGRIMYTVFEHTFHVREGDEQRTFFSFPA |
| | | VVAPFKCSVLPLSQNQEFMPFVKELSEALTRHGVSHKVDD |
| | | SSGSIGRRYARTDEIGVAFGVTIDFDTVNKTPHTATLRDRDS |
| | | MRQIRAEISELPSIVQDLANGNITWADVEARYPLFEGQETG |
| | | KKETIEE |
| | | SEQ ID NO: 35 |
| Metalloproteinase | TIMP 1 | MAPFEPLASGILLLLWLIAPSRACTCVPPHPQTAFCNSDLVI |
| inhibitor 1 | | RAKFVGTPEVNQTTLYQRYEIKMTKMYKGFQALGDAADIR |
| | | FVYTPAMESVCGYFHRSHNRSEEFLIAGKLQDGLLHITTCSF |
| | | VAPWNSLSLAQRRGFTKTYTVGCEECTVFPCLSIPCKLQSG |
| | | THCLWTDQLLQGSEKGFQSRHLACLPREPGLCTWQSLRSQI |
| | | A |
| | | SEQ ID NO: 36 |
| Serotransferrin | TRFE | MRLAVGALLVCAVLGLCLAVPDKTVRWCAVSEHEATKCQ |
| | | SFRDHMKSVIPSDGPSVACVKKASYLDCIRAIAANEADAVT |
| | | LDAGLVYDAYLAPNNLKPVVAEFYGSKEDPQTFYYAVAV |
| | | VKKDSGFQMNQLRGKKSCHTGLGRSAGWNIPIGLLYCDLP |
| | | EPRKPLEKAVANFFSGSCAPCADGTDFPQLCQLCPGCGCST |
| | | LNQYFGYSGAFKCLKDGAGDVAFVKHSTIFENLANKADRD |
| | | QYELLCLDNTRKPVDEYKDCHLAQVPSHTVVARSMGGKE |
| | | DLIWELLNQAQEHFGKDKSKEFQLFSSPHGKDLLFKDSAH |
| | | GFLKVPPRMDAKMYLGYEYVTAIRNLREGTCPEAPTDECK |
| | | PVKWCALSHHERLKCDEWSVNSVGKIECVSAETTEDCIAKI |
| | | MNGEADAMSLDGGFVYIAGKCGLVPVLAENYNKSDNCED |
| | | TPEAGYFAIAVVKKSASDLTWDNLKGKKSCHTAVGRTAG |
| | | WNIPMGLLYNKINHCRFDEFFSEGCAPGSKKDSSLCKLCM |
| | | GSGLNLCEPNNKEGYYGYTGAFRCLVEKGDVAFVKHQTV |
| | | PQNTGGKNPDPWAKNLNEKDYELLCLDGTRKPVEEYANC |
| | | HLARAPNHAVVTRKDKEACVHKILRQQQHLFGSNVTDCSG |
| | | NFCLFRSETKDLLFRDDTVCLAKLHDRNTYEKYLGEEYVK |
| | | AVGNLRKCSTSSLLEACTFRRP |
| | | SEQ ID NO: 37 |

The biomarkers can include polypeptides comprising an amino acid sequence having at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identity to any of the amino acid sequences described herein. The biomarkers can include polypeptides comprising an amino acid sequence having at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identity over a length of 5 or more, 6 or more, 7 or more, 8 or more, 9 or more, 10 or more, 11 or more, 12 or more, 13 or more, 14 or more, 15 or more, 16 or more, 17 or more, 18 or more, 19 or more, 20 or more, 21 or more, 22 or more, 23 or more, 24 or more, 25 or more, 26 or more, 27 or more, 28 or more, 29 or more, 20 or more, 31 or more, 32 or more, 33 or more, 34 or more, 35 or more, 36 or more, 37 or more, 38 or more, 39 or more, 40 or more, 41 or more, 42 or more, 43 or more, 44 or more, 45 or more, 46 or more, 47 or more, 48 or more, 49 or more, 50 or more, 51 or more, 52 or more, 53 or more, 54 or more, 55 or more, 56 or more, 57 or more, 58 or more, 59 or more, 60 or more, 61 or more, 62 or more, 63 or more, 64 or more, 65 or more, 66 or more, 67 or more, 68 or more, 69 or more, 70 or more, 71 or more, 72 or more, 73 or more, 74 or more, 75 or more, 76 or more, 77 or more, 78 or more, 79 or more, 80 or more, 81 or more, 82 or more, 83 or more, 84 or more, 85 or more, 86 or more, 87 or more, 88 or more, 89 or more, 90 or more, 91 or more, 92 or more, 93 or more, 94 or more, 95 or more, 96 or more, 97 or more, 98 or more, 99 or more, or 100 or more continuous amino acid residues of any of the sequences described herein.

Biomarkers described herein can also include nucleic acids encoding a polypeptide with an amino acid sequence having at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% homology to any of the amino acid sequences described herein, and all modified forms and/or fragments thereof. Modified forms of the biomarker include for example any splice-variants of the disclosed biomarkers and their corresponding RNA or DNA which encode them. In certain cases the modified forms, fragments, or their corresponding RNA or DNA, may exhibit better discriminatory power in diagnosis than the full-length protein.

Biomarkers described herein can also include truncated forms or polypeptide fragments of any of the proteins described herein. Truncated forms or polypeptide fragments of a protein can include N-terminally deleted or truncated forms and C-terminally deleted or truncated forms. Truncated forms or fragments of a protein can include fragments arising by any mechanism, such as, without limitation, by alternative translation, exo- and/or endo-proteolysis and/or degradation, for example, by physical, chemical and/or enzymatic proteolysis. Without limitation, a truncated or fragment of a protein, polypeptide or peptide may represent less or more than 1%, less or more than 15%, or at least about 10%, for example, >20%, >30% or >40%, such as >50%, for example, >60%, >70%, or >80%, or even 90% or >95% of the amino acid sequence of the protein.

Without limitation, a truncated or fragment of a protein may include a sequence of about 5-20 consecutive amino acids, or about 10-50 consecutive amino acids, or about 20-100 consecutive amino acids, or about 30-150 consecutive amino acids, or about 50-500 consecutive amino acids, or about 200-1000 consecutive amino acids, or more than 1000 consecutive amino acids of the corresponding full length protein.

In some instances, a fragment may be N-terminally and/or C-terminally truncated by between 1 and about 20 amino acids, such as, for example, by between 1 and about 15 amino acids, or by between 1 and about 10 amino acids, or by between 1 and about 5 amino acids, compared to the corresponding mature, full-length protein or its soluble or plasma circulating form.

Any protein biomarker of the present disclosure such as a peptide, polypeptide or protein and fragments thereof may also encompass modified forms of said marker, peptide, polypeptide or protein and fragments such as bearing post-expression modifications including but not limited to, modifications such as phosphorylation, glycosylation, lipidation, methylation, cysteinylation, sulphonation, glutathionylation, acetylation, oxidation of methionine to methionine sulphoxide or methionine sulphone, and the like.

In some instances, a fragmented protein may be N-terminally and/or C-terminally truncated. Such fragmented protein can comprise one or more, or all transitional ions of the N-terminally (a, b, c-ion) and/or C-terminally (x, y, z-ion) truncated protein or peptide. Exemplary human markers, nucleic acids, proteins or polypeptides as taught herein may be as annotated under NCBI Genbank (http://www.ncbi.nlm.nih.gov/) or Swissprot/Uniprot (http://www.uniprot.org/) accession numbers. In some instances said sequences may be of precursors (for example, preproteins) of the of markers, nucleic acids, proteins or polypeptides as taught herein and may include parts which are processed away from mature molecules. In some instances although only one or more isoforms may be disclosed, all isoforms of the sequences are intended.

In some embodiments, a diagnostic method provided herein comprises measuring in the biological sample a biomarker panel comprising at least two biomarkers selected from the group consisting of A1AG1, A1AT, AACT, AMY2B, ANXA1, APOA1, CAH1, CATD, CATS, CEAM3, CLUS, CTNB1, CO3, CO9, CRP, CSF1, DPP4, ECH1, FHL1, FIBB, FIBG, FRIL, FUCO, GELS, HPT, OSTP, PRDX1, SAA1, SAHH, SBP1, SEPR, SPB6, SPON2, SYG, TIMP1, and TRFE. In an alternative embodiment, a diagnostic method provided herein comprises measuring in the biological sample a biomarker panel consisting of A1AG1, AACT, CO3, CO9, and SAM.

In an some embodiments, a diagnostic method provided herein comprises measuring in the biological sample a biomarker panel comprising at least two biomarkers selected from the group consisting of A1AG1, A1AT, AACT, APOA1, CATD, CEAM3, CLUS, CO3, CO9, CRP, FIBB, FIBG, GELS, OSTP, PRDX1, SAA1, SBP1, and SEPR. In some embodiments, a diagnostic method provided herein comprises measuring in the biological sample a biomarker panel consisting of A1AG1, A1AT, CATD, CEAM3, CO9, OSTP, and SEPR. In some embodiments, a diagnostic method provided herein comprises measuring in the biological sample a biomarker panel consisting of A1AG1, A1AT, APOA1, CATD, CEAM3, CLUS, CO3, CO9, FIBB, FIBG, GELS, PRDX1, SBP1, and SEPR. In some embodiments, a diagnostic method provided herein comprises measuring in the biological sample a biomarker panel consisting of A1AG1, A1AT, CATD, CEAM3, CO9, and SEPR. In some embodiments, a diagnostic method provided herein comprises measuring in the biological sample a biomarker panel consisting of A1AG1, A1AT, AACT, CATD, CEAM3, CO9, CRP, GELS, SAA1, and SEPR. In some embodiments, a diagnostic method provided herein comprises measuring in the biological sample a biomarker panel consisting of CATD, CEA, CO3, CO9, GELS, and SEPR. Any of the method described herein can comprise comparing the amount of each of the at least two biomarkers in the biological sample to a reference amount of each of the at least two biomarkers. Any of the method described herein can comprise comparing the profile of the biomarker panel in a subject to a reference profile of the biomarker panel. The reference amount can be an amount of the biomarker in a control subject. The reference profile of the biomarker panel can be a biomarker profile of a control subject. The control subject can be a subject having a known diagnosis. For example, the control subject can be a negative control subject. The negative control subject can be a subject that does not have advanced colorectal adenoma. The negative control subject can be a subject that does not have CRC. The negative control subject can be a subject that does not have a colon polyp. For other example, the control subject can be a positive control subject. The positive control subject can be a subject having a confirmed diagnosis of advanced colorectal adenoma. The positive control subject can be a subject having a confirmed diagnosis of CRC. The positive control subject can be a subject having a confirmed diagnosis of any stage of CRC (for example, Stage 0, Stage I, Stage II, Stage HA, Stage IIB, Stage IIC, Stage III, Stage IIIA, Stage IIIB, Stage IIIC, Stage IV, Stage IVA, or Stage IVB). The reference amount can be a predetermined level of the biomarker, wherein the predetermined level is set based upon a measured amount of the biomarker in a control subject.

In some cases, comparing comprises determining a difference between an amount of the biomarker in the biological sample obtained from the subject and the reference amount of the biomarker. The method can, for example, comprise detecting a presence or absence of at least one of advanced colorectal adenoma and CRC based upon a deviation (for example, measured difference) of the amount of at least one of the measured biomarkers in the biological sample obtained from the subject as compared to a reference amount of the at least one measured biomarkers. In some examples, the method comprises detecting a presence of at least one of advanced colorectal adenoma and CRC if the deviation of the amount of the at least one measured biomarker from the biological sample obtained from the subject as compared to a positive reference value (for example, an amount of the measured biomarker from a positive control subject) is low. For other example, the method comprises detecting a presence of at least one of advanced colorectal adenoma and CRC if the deviation of the amount of the at least one measured biomarker from the biological sample obtained from the subject as compared to a negative reference value (for example, measured from a negative control subject) is high. In some examples, the method comprises detecting an absence of at least one of advanced colorectal adenoma and CRC if the deviation of the amount of the at least one measured biomarker from the biological sample obtained from the subject as compared to a positive reference value (for example, measured from a positive control subject) is high. In some examples, the method comprises detecting an absence of at least one of advanced colorectal adenoma and CRC if the deviation of the amount of the at least one measured biomarker from the biological sample obtained from the subject as compared to a negative reference value (for example, measured from a negative control subject) is low. In some cases, detection of a presence or absence of at least one of advanced colorectal adenoma and CRC can be based upon a clinical outcome score produced by an algorithm described herein. The algorithm can be used for assessing the deviation between an amount of a measured biomarker in the biological sample obtained from the subject and a reference amount of the biomarker.

In practicing any of the methods described herein, comparing can comprise determining a difference between a biomarker profile of a subject to a reference biomarker profile. The method can, for example, comprise detecting a presence or absence of at least one of advanced colorectal adenoma and CRC based upon a deviation (for example, measured difference) of the biomarker profile of the subject as compared to a reference biomarker profile. For example, the method can comprise detecting a presence of at least one of advanced colorectal adenoma and CRC if the deviation of the biomarker profile of the subject as compared to a positive reference biomarker profile (for example, a biomarker profile based upon measurements of panel biomarkers from a positive control subject) is low. For other example, the method can comprise detecting a presence of at least one of advanced colorectal adenoma and CRC if the deviation oft the biomarker profile of the subject as compared to a negative reference biomarker profile (for example, a biomarker profile based upon measurements of panel biomarkers from a negative control subject) is high. In some cases, the method comprises detecting an absence of at least one of advanced colorectal adenoma and CRC if the deviation of the biomarker profile of the subject as compared to a positive reference biomarker profile is high. In some examples, the method comprises detecting an absence of at least one of advanced colorectal adenoma and CRC if the deviation of the biomarker profile of the subject as compared to a negative reference biomarker profile is low. In some cases, detection of a presence or absence of at least one of advanced colorectal adenoma and CRC can be based upon a clinical outcome score produced by an algorithm described herein. The algorithm can be used for assessing the deviation between the biomarker profile of the subject to a reference biomarker profile.

In some embodiments, the method comprises detecting a presence or absence of an advanced colorectal adenoma in the subject. The advanced colorectal adenoma can be a colorectal advanced colorectal adenoma. The methods described herein can be used to detect a presence or absence of an advanced colorectal adenoma having a dimension that is greater than 1 cm. The methods described herein can be used to detect a presence or absence of an advanced colorectal adenoma of villous character. In some cases, a diagnostic method provided herein comprises measuring a biomarker panel comprising at least two biomarkers in the biological sample, wherein the at least two biomarkers comprise CATD and FUCO. In particular cases, such diagnostic method comprises measuring a biomarker panel three biomarkers in the biological sample. The three biomarkers can be, for example, CATD, CATS, and FUCO. The three biomarkers can be CATD, FUCO, and FIBB. The three biomarkers can be CATD, FUCO, and SAHH. In some cases, such diagnostic method comprises measuring a biomarker panel comprising four biomarkers in the biological sample. The four biomarkers can be, for example, CATD, FIBB, FUCO, and SAHH. In some cases, the method comprises providing a positive diagnosis of advanced colorectal adenoma if a deviation in the level of at least one of CATD, FUCO, FIBB, and SAHH in the biological sample obtained from the subject as compared to a positive reference value is low. In some cases, the method comprises providing a positive diagnosis of advanced colorectal adenoma if a deviation in the level of at least one of CATD, FUCO, FIBB, and SAHH in the biological sample obtained from the subject as compared to a negative reference value is high. In some cases, the method comprises providing a positive diagnosis of advanced colorectal adenoma if a deviation in the level of at least one of CATD, FUCO, FIBB, and SAHH in the biological sample obtained from the subject as compared to a positive reference value is high. In some cases, the method comprises providing a positive diagnosis of advanced colorectal adenoma if a deviation in the level of at least one of CATD, FUCO, FIBB, and SAHH in the biological sample obtained from the subject as compared to a negative reference value is low. Such diagnostic method can detect advanced colorectal adenoma with a sensitivity greater than 50%, greater than 55%, greater than 60%, greater than 65%, greater than 70%, greater than 75%, greater than 80%, greater than 85%, greater than 90%, greater than 95%, greater than 96%, greater than 97%, greater than 98%, greater than 99%, or about 100%. Such diagnostic method can detect advanced colorectal adenoma with a sensitivity that is between about 50%-100%, between about 60%-100%, between about 70%-100%, between about 80%-100%, or between about 90-100%. Such diagnostic method can detect advanced colorectal adenoma with a specificity greater than 50%, greater than 55%, greater than 60%, greater than 65%, greater than 70%, greater than 75%, greater than 80%, greater than 85%, greater than 90%, greater than 95%, greater than 96%, greater than 97%, greater than 98%, greater than 99%, or about 100%. Such diagnostic method can detect advanced colorectal adenoma with a specificity that is between about 50%-100%, between about 60%-100%, between about 70%-100%, between about 80%-100%, or between about 90-100%. In particular embodiments, such diagnostic method can detect advanced colorectal adenoma with a sensitivity and a specificity that is 50% or greater, 60% or greater, 70% or greater, 75% or greater, 80% or greater, 85% or greater, 90% or greater. In particular embodiments, such diagnostic can detect advanced colorectal adenoma with a sensitivity and a specificity that is between about 50%-100%, between about 60%-100%, between about 70%-100%, between about 80%-100%, or between about 90-100%.

In some embodiments, a biomarker panel comprises at least two biomarkers which are CO9 and GELS. Such diagnostic method can be used for detection of CRC in the subject. In some embodiments, no more than two biomarkers which are CO9 and GELS are measured in the biological sample. In some cases, the method comprises providing a positive diagnosis of CRC if a deviation in the level of at least one of CO9 and GELS in the biological sample obtained from the subject as compared to a positive reference value is low. In some cases, the method comprises providing a positive diagnosis of CRC if a deviation in the level of at least one of CO9 and GELS in the biological sample obtained from the subject as compared to a negative reference value is high. In some cases, the method comprises providing a positive diagnosis of CRC if a deviation in the level of at least one of CO9 and GELS in the biological sample obtained from the subject as compared to a positive reference value is high. In some cases, the method comprises providing a positive diagnosis of CRC if a deviation in the level of at least one of CO9 and GELS in the biological sample obtained from the subject as compared to a negative reference value is low.

In some cases the at least two biomarkers in the panel comprise CRP and TIMP1. Such biomarker panel can be used for detection of CRC in the subject. In some embodiments, no more than two biomarkers which are CRP and TIMP1 are measured in the biological sample. In some cases, the method comprises providing a positive diagnosis of CRC if a deviation in the level of at least one of CRP and TIMP1 in the biological sample obtained from the subject as compared to a positive reference value is low. In some cases, the method comprises providing a positive diagnosis of CRC if a deviation in the level of at least one of CRP and TIMP1 in the biological sample obtained from the subject as compared to a negative reference value is high. In some cases, the method comprises providing a positive diagnosis of CRC if a deviation in the level of at least one of CRP and TIMP1 in the biological sample obtained from the subject as compared to a positive reference value is high. In some cases, the method comprises providing a positive diagnosis of CRC if a deviation in the level of at least one of CRP and TIMP1 in the biological sample obtained from the subject as compared to a negative reference value is low.

In some cases, a diagnostic method provided herein comprises measuring a biomarker panel comprising three biomarkers in the biological sample. The three biomarkers can be AACT, CO9, and SYG. In some cases, the method comprises providing a positive diagnosis of CRC if a deviation in the level of at least one of AACT, CO9, and SYG in the biological sample obtained from the subject as compared to a positive reference value is low. In some cases, the method comprises providing a positive diagnosis of CRC if a deviation in the level of at least one of AACT, CO9, and SYG in the biological sample obtained from the subject as compared to a negative reference value is high. In some cases, the method comprises providing a positive diagnosis of CRC if a deviation in the level of at least one of AACT, CO9, and SYG in the biological sample obtained from the subject as compared to a positive reference value is high. In some cases, the method comprises providing a positive diagnosis of CRC if a deviation in the level of at least one of AACT, CO9, and SYG in the biological sample obtained from the subject as compared to a negative reference value is low. In some embodiments, no more than three biomarkers which are AACT, CO9, and SYG are measured in the biological sample.

In some cases, a diagnostic method provided herein for detection of CRC comprises measuring a biomarker panel comprising four biomarkers in the biological sample. In some embodiments more than four biomarkers are measured in the biological sample. In some embodiments no more than four biomarkers are measured in the biological sample.

In some embodiments, the four biomarkers are CO9, GELS, PRDX1, and CATD In some cases, the method comprises providing a positive diagnosis of CRC if a deviation in the level of at least one of CO9, GELS, PRDX1, and CATD in the biological sample obtained from the subject as compared to a positive reference value is low. In some cases, the method comprises providing a positive diagnosis of CRC if a deviation in the level of at least one of CO9, GELS, PRDX1, and CATD in the biological sample obtained from the subject as compared to a negative reference value is high. In some cases, the method comprises providing a positive diagnosis of CRC if a deviation in the level of at least one of CO9, GELS, PRDX1, and CATD in the biological sample obtained from the subject as compared to a positive reference value is high. In some cases, the method comprises providing a positive diagnosis of CRC if a deviation in the level of at least one of CO9, GELS, PRDX1, and CATD in the biological sample obtained from the subject as compared to a negative reference value is low.

In some embodiments, the four biomarkers are A1AT, APOA1, FIBB, and CEAM3. In some cases, the method comprises providing a positive diagnosis of CRC if a deviation in the level of at least one of A1AT, APOA1, FIBB, and CEAM3 in the biological sample obtained from the subject as compared to a positive reference value is low. In some cases, the method comprises providing a positive diagnosis of CRC if a deviation in the level of at least one of A1AT, APOA1, FIBB, and CEAM3 in the biological sample obtained from the subject as compared to a negative reference value is high. In some cases, the method comprises providing a positive diagnosis of CRC if a deviation in the level of at least one of A1AT, APOA1, FIBB, and CEAM3 in the biological sample obtained from the subject as compared to a positive reference value is high. In some cases, the method comprises providing a positive diagnosis of CRC if a deviation in the level of at least one of A1AT, APOA1, FIBB, and CEAM3 in the biological sample obtained from the subject as compared to a negative reference value is low.

In some embodiments, the four biomarkers are CAH1, CRP, FIBG, and CTNB1. In some cases, the method comprises providing a positive diagnosis of CRC if a deviation in the level of at least one of CAH1, CRP, FIBG, and CTNB1 in the biological sample obtained from the subject as compared to a positive reference value is low. In some cases, the method comprises providing a positive diagnosis of CRC if a deviation in the level of at least one of CAH1, CRP, FIBG, and CTNB1 in the biological sample obtained from the subject as compared to a negative reference value is high. In some cases, the method comprises providing a positive diagnosis of CRC if a deviation in the level of at least one of CAH1, CRP, FIBG, and CTNB1 in the biological sample obtained from the subject as compared to a positive reference value is high. In some cases, the method comprises providing a positive diagnosis of CRC if a deviation in the level of at least one of CAH1, CRP, FIBG, and CTNB1 in the biological sample obtained from the subject as compared to a negative reference value is low.

In some embodiments, the four biomarkers are A1AG1, A1AT, CO9, and GELS. In some cases, the method comprises providing a positive diagnosis of CRC if a deviation in the level of at least one of A1AG1, A1AT, CO9, and GELS in the biological sample obtained from the subject as compared to a positive reference value is low. In some cases, the method comprises providing a positive diagnosis of CRC if a deviation in the level of at least one of A1AG1, A1AT, CO9, and GELS in the biological sample obtained from the subject as compared to a negative reference value is high. In some cases, the method comprises providing a positive diagnosis of CRC if a deviation in the level of at least one of A1AG1, A1AT, CO9, and GELS in the biological sample obtained from the subject as compared to a positive reference value is high. In some cases, the method comprises providing a positive diagnosis of CRC if a deviation in the level of at least one of A1AG1, A1AT, CO9, and GELS in the biological sample obtained from the subject as compared to a negative reference value is low.

In some embodiments the method can comprising measuring a biomarker panel comprising more than four biomarkers are measured in the biological sample. Particular embodiments of a diagnostic method described herein comprise measuring a biomarker panel, wherein the biomarker panel comprises more than four biomarkers in the biological sample, wherein the more than four biomarkers comprise A1AG1, A1AT, CO9, and GELS. For example, a biomarker panel can comprise 5, 6, 7, 8, 9, 10, 11, 12, 13, 14, 15, 16, 17, 18, 19, 20, or more than 20 biomarkers, wherein the 5, 6, 7, 8, 9, 10, 11, 12, 13, 14, 15, 16, 17, 18, 19, 20, or more than 20 biomarkers comprise A1AG1, A1AT, CO9, and GELS. In some cases, the biomarker panel comprises 5, 6, 7, 8, 9, 10, 11, 12, 13, 14, 15, 16, 17, 18, 19, 20, or more than 20 biomarkers including A1AG1, A1AT, CO9, and GELS, wherein the 5, 6, 7, 8, 9, 10, 11, 12, 13, 14, 15, 16, 17, 18, 19, 20, or more than 20 biomarkers including A1AG1, A1AT, CO9, and GELS also include at least one of: AACT, ANXA1, APOL1, CRP, CSF1, FHL1, FIBG, HPT, SA11, AMY2B, CLUS, ECH1, FRIL, OSTP, SBP1, SEPR, SPON2, and TIMP1. In some cases, the biomarker panel comprises 5-20, 8-16, or 10-15 biomarkers, including A1AG1, A1AT, CO9, and GELS. In some cases, the biomarker panel comprises 13 biomarkers. In particular cases, the 13 biomarkers are A1AG1, A1AT, AACT, ANXA1, APOA1, CO9, CRP, CSF1, FHL1, FIBG, GELS, HPT, and SAM. In some cases, the method comprises providing a positive diagnosis of CRC if a deviation in the level of at least one of A1AG1, A1AT, AACT, ANXA1, APOA1, CO9, CRP, CSF1, FHL1, FIBG, GELS, HPT, and SAM in the biological sample obtained from the subject as compared to a positive reference value is low. In some cases, the method comprises providing a positive diagnosis of CRC if a deviation in the level of at least one of A1AG1, A1AT, AACT, ANXA1, APOA1, CO9, CRP, CSF1, FHL1, FIBG, GELS, HPT, and SAM in the biological sample obtained from the subject as compared to a negative reference value is high. In some cases, the method comprises providing a positive diagnosis of CRC if a deviation in the level of at least one of A1AG1, A1AT, AACT, ANXA1, APOA1, CO9, CRP, CSF1, FHL1, FIBG, GELS, HPT, and SAA1 in the biological sample obtained from the subject as compared to a positive reference value is high. In some cases, the method comprises providing a positive diagnosis of CRC if a deviation in the level of at least one of A1AG1, A1AT, AACT, ANXA1, APOA1, CO9, CRP, CSF1, FHL1, FIBG, GELS, HPT, and SAM in the biological sample obtained from the subject as compared to a negative reference value is low.

In some cases, the 13 biomarkers are A1AG1, A1AT, AMY2B, CLUS, CO9, ECH1, FRIL, GELS, OSTP, SBP1, SEPR, SPON2, and TIMP1. Such biomarker panel can be used for detection of CRC in the subject. In some cases, the method comprises providing a positive diagnosis of CRC if a deviation in the level of at least one of A1AG1, A1AT, AMY2B, CLUS, CO9, ECH1, FRIL, GELS, OSTP, SBP1, SEPR, SPON2, and TIMP1 in the biological sample obtained from the subject as compared to a positive reference value is low. In some cases, the method comprises providing a positive diagnosis of CRC if a deviation in the level of at least one of A1AG1, A1AT, AMY2B, CLUS, CO9, ECH1, FRIL, GELS, OSTP, SBP1, SEPR, SPON2, and TIMP1 in the biological sample obtained from the subject as compared to a negative reference value is high. In some cases, the method comprises providing a positive diagnosis of CRC if a deviation in the level of at least one of A1AG1, A1AT, AMY2B, CLUS, CO9, ECH1, FRIL, GELS, OSTP, SBP1, SEPR, SPON2, and TIMP1 in the biological sample obtained from the subject as compared to a positive reference value is high. In some cases, the method comprises providing a positive diagnosis of CRC if a deviation in the level of at least one of A1AG1, A1AT, AMY2B, CLUS, CO9, ECH1, FRIL, GELS, OSTP, SBP1, SEPR, SPON2, and TIMP1 in the biological sample obtained from the subject as compared to a negative reference value is low.

In some cases, a diagnostic method provided herein comprises measuring a biomarker panel comprising five biomarkers in the biological sample. The five biomarkers can be AACT, CO3, CO9, CRP, and GELS. In some cases, the method comprises providing a positive diagnosis of CRC if a deviation in the level of at least one of AACT, CO3, CO9, CRP, and GELS in the biological sample obtained from the subject as compared to a positive reference value is low. In some cases, the method comprises providing a positive diagnosis of CRC if a deviation in the level of at least one of AACT, CO3, CO9, CRP, and GELS in the biological sample obtained from the subject as compared to a negative reference value is high. In some cases, the method comprises providing a positive diagnosis of CRC if a deviation in the level of at least one of AACT, CO3, CO9, CRP, and GELS in the biological sample obtained from the subject as compared to a positive reference value is high. In some cases, the method comprises providing a positive diagnosis of CRC if a deviation in the level of at least one of AACT, CO3, CO9, CRP, and GELS in the biological sample obtained from the subject as compared to a negative reference value is low.

The five biomarkers can be A1AT, CO3, FIBG, GELS, and SPB6. In some cases, the method comprises providing a positive diagnosis of CRC if a deviation in the level of at least one of A1AT, CO3, FIBG, GELS, and SPB6 in the biological sample obtained from the subject as compared to a positive reference value is low. In some cases, the method comprises providing a positive diagnosis of CRC if a deviation in the level of at least one of A1AT, CO3, FIBG, GELS, and SPB6 in the biological sample obtained from the subject as compared to a negative reference value is high. In some cases, the method comprises providing a positive diagnosis of CRC if a deviation in the level of at least one of A1AT, CO3, FIBG, GELS, and SPB6 in the biological sample obtained from the subject as compared to a positive reference value is high. In some cases, the method comprises providing a positive diagnosis of CRC if a deviation in the level of at least one of A1AT, CO3, FIBG, GELS, and SPB6 in the biological sample obtained from the subject as compared to a negative reference value is low.

The five biomarkers can be CRP, DPP4, SBP1, SEPR, and SRC. In some cases, the method comprises providing a positive diagnosis of CRC if a deviation in the level of at least one of CRP, DPP4, SBP1, SEPR, and SRC in the biological sample obtained from the subject as compared to a positive reference value is low. In some cases, the method comprises providing a positive diagnosis of CRC if a deviation in the level of at least one of CRP, DPP4, SBP1, SEPR, and SRC in the biological sample obtained from the subject as compared to a negative reference value is high. In some cases, the method comprises providing a positive diagnosis of CRC if a deviation in the level of at least one of CRP, DPP4, SBP1, SEPR, and SRC in the biological sample obtained from the subject as compared to a positive reference value is high. In some cases, the method comprises providing a positive diagnosis of CRC if a deviation in the level of at least one of CRP, DPP4, SBP1, SEPR, and SRC in the biological sample obtained from the subject as compared to a negative reference value is low. In some cases wherein the five biomarkers are CRP, DPP4, SBP1, SEPR, and SRC, the subjects are male.

In some embodiments, a biomarker panel comprises no more than five biomarkers. In some embodiments more than five biomarkers are measured in the biological sample. Such diagnostic methods and biomarker panels can be used for detection of CRC in the subject.

In some cases, a panel comprises a ratio of a level of a first biomarker to a level of a second biomarker. Accordingly, in some cases, a diagnostic method provided herein comprises determining a ratio of a level of the first biomarker to a level of the second biomarker in the biological sample obtained from the subject. In some cases, the method comprises providing a positive diagnosis of CRC if a deviation in the ratio of the first biomarker to the second biomarker in the biological sample obtained from the subject as compared to a positive reference value is low. In some cases, the method comprises providing a positive diagnosis of CRC if a deviation in the ratio of the first biomarker to the second biomarker in the biological sample obtained from the subject as compared to a negative reference value is high. In some cases, the method comprises providing a positive diagnosis of if a deviation in the ratio of the first biomarker to the second biomarker in the biological sample obtained from the subject as compared to a positive reference value is high. In some cases, the method comprises providing a positive diagnosis of CRC if a deviation in the ratio of the first biomarker to the second biomarker in the biological sample obtained from the subject as compared to a negative reference value is low.

In some cases, the first biomarker is A1AT and the second biomarker is TRFE. In some cases wherein the first biomarker is A1AT and the second biomarker is TRFE, the subject is male. Such diagnostic method can be used for detection of CRC in the subject. In some cases, the method comprises providing a positive diagnosis of CRC if a deviation in the ratio of A1AT to TRFE in the biological sample obtained from the subject as compared to a positive reference value is low. In some cases, the method comprises providing a positive diagnosis of CRC if a deviation in the ratio of A1AT to TRFE in the biological sample obtained from the subject as compared to a negative reference value is high. In some cases, the method comprises providing a positive diagnosis of if a deviation in the ratio of A1AT to TRFE in the biological sample obtained from the subject as compared to a positive reference value is high. In some cases, the method comprises providing a positive diagnosis of CRC if a deviation in the ratio of A1AT to TRFE in the biological sample obtained from the subject as compared to a negative reference value is low. In some cases, the method comprises providing a positive diagnosis of CRC if a deviation in the ratio of A1AT to TRFE in the biological sample obtained from the subject as compared to a positive reference value is low and the subject is male. In some cases, the method comprises providing a positive diagnosis of CRC if a deviation in the ratio of A1AT to TRFE in the biological sample obtained from the subject as compared to a negative reference value is high and the subject is male. In some cases, the method comprises providing a positive diagnosis of if a deviation in the ratio of A1AT to TRFE in the biological sample obtained from the subject as compared to a positive reference value is high and the subject is male. In some cases, the method comprises providing a positive diagnosis of CRC if a deviation in the ratio of A1AT to TRFE in the biological sample obtained from the subject as compared to a negative reference value is low and the subject is male.

In some cases, the first biomarker is APOA1. In some cases wherein the first biomarker is APOA1, the second biomarker is selected from the group consisting of CO3, CO9, A1AT, and FIBG. For example, a method provided herein can comprise at least one of: determining a ratio of APOA1 to CO3, determining a ratio of APOA1 to CO9, determining a ratio of A1AT to APOA1, and determining a ratio of APOA1 to FIBG. In some cases, the method further comprises determining a second ratio, wherein the second ratio is a ratio of APOA1 to a level of a third biomarker in the biological sample of the subject. In some cases the third biomarker is selected from the group consisting of CO3, CO9, A1AT, and FIBG. For example, a method provided herein can comprise determining a ratio of APOA1 to CO3 and a ratio of APOA1 to CO9. For other example, a method provided herein can comprise determining a ratio of A1AT to APOA1 and a ratio of APOA1 to FIBG. In some cases, the method comprises providing a positive diagnosis of CRC if a deviation in at least one of the first ratio and second ratio in the biological sample obtained from the subject as compared to a positive reference value is low. In some cases, the method comprises providing a positive diagnosis of CRC if a deviation in at least one of the first ratio and second ratio in the biological sample obtained from the subject as compared to a negative reference value is high. In some cases, the method comprises providing a positive diagnosis of if a deviation in at least one of the first ratio and second ratio in the biological sample obtained from the subject as compared to a positive reference value is high. In some cases, the method comprises providing a positive diagnosis of CRC if a deviation in at least one of the first ratio and second ratio in the biological sample obtained from the subject as compared to a negative reference value is low.

Any of the diagnostic methods described herein for detection of CRC in a subject can detect CRC with a sensitivity greater than 50%, greater than 55%, greater than 60%, greater than 65%, greater than 70%, greater than 75%, greater than 80%, greater than 85%, greater than 90%, greater than 95%, greater than 96%, greater than 97%, greater than 98%, greater than 99%, or about 100%. Such diagnostic methods can detect CRC with a sensitivity that is between about 50%-100%, between about 60%-100%, between about 70%-100%, between about 80%-100%, or between about 90-100%. Such diagnostic methods can detect CRC with a specificity greater than 50%, greater than 55%, greater than 60%, greater than 65%, greater than 70%, greater than 75%, greater than 80%, greater than 85%, greater than 90%, greater than 95%, greater than 96%, greater than 97%, greater than 98%, greater than 99%, or about 100%. Such diagnostic methods can detect CRC with a specificity that is between about 50%-100%, between about 60%-100%, between about 70%-100%, between about 80%-100%, or between about 90-100%. In particular embodiments, such diagnostic methods can detect CRC with a sensitivity and a specificity that is 50% or greater, 60% or greater, 70% or greater, 75% or greater, 80% or greater, 85% or greater, 90% or greater. In particular embodiments, such diagnostic methods can detect CRC with a sensitivity and a specificity that is between about 50%-100%, between about 60%-100%, between about 70%-100%, between about 80%-100%, or between about 90-100%.

### Exemplary Subjects

Biological samples can be collected from subjects who want to determine their likelihood of having at least one of advanced colorectal adenoma and CRC. The subject can be healthy and asymptomatic. The subject can be of any age. For example, the subject can be between the ages of 0 to about 30 years, about 20 to about 50 years, about 40 to about 100 years, or over 100 years. In various embodiments, the subject is healthy, asymptomatic and between the ages of 0-30 years, 20-50 years, 40-100 years, or over 100 years. The subject can be at least 30 years of age, at least 40 years of age, or at least 50 years of age. The subject can be less than 50 years of age, less than 40 years of age, or less than 30 years of age. In various embodiments, the subject is healthy and asymptomatic. In various embodiments, the subject has no family history of at least one of: CRC, adenoma, and polyps. In various embodiments, the subject has not had a colonoscopy, sigmoidoscopy, or colon tissue biopsy. In various embodiments, the subject is healthy and asymptomatic and has not received a colonoscopy, sigmoidoscopy, or colon tissue biopsy. In some cases, the subject may not have received a colonoscopy, sigmoidoscopy, or colon tissue biopsy and has one or more of: a symptom of CRC, a family history of CRC, and a risk factor for CRC. In some cases, a biological sample can be obtained from a subject during routine examination, or to establish baseline levels of the biomarkers. In some cases, a subject may have no symptoms for colorectal carcinoma, may have no family history for colorectal carcinoma, and/or may have no recognized risk factors for colorectal carcinoma.

In some cases, a subject may have at least one of: a symptom for colorectal carcinoma, a family history for colorectal carcinoma, and a recognized risk factor for colorectal carcinoma. In some cases, a subject may be identified through screening assays (for example, fecal occult blood testing or sigmoidoscopy) or rectal digital exam or rigid or flexible colonoscopy or CT scan or other x-ray techniques as being at high risk for or having CRC. For example, one or more methods described herein may be applied to a subject undergoing treatment for CRC, to determine the effectiveness of the therapy or treatment they are receiving.

### Exemplary Biological Samples

Exemplary biological samples can include one or more of, but are not limited to: urine, stool, tears, whole blood, serum, plasma, blood constituent, bone marrow, tissue, cells, organs, saliva, cheek swab, lymph fluid, cerebrospinal fluid, lesion exudates and other fluids produced by the body. The biological sample can be a solid biological sample, for example, a tissue biopsy. The biopsy can be fixed, paraffin embedded, or fresh.

Biological samples may be processed using any means known in the art or otherwise described herein in order to enable measurement of one or more biomarkers as described herein. Sample preparation operations may comprise, for example, extraction and/or isolation of intracellular material from a cell or tissue such as the extraction of nucleic acids, protein, or other macromolecules. Sample preparation which can be used with the methods of disclosure include but are not limited to, centrifugation, affinity chromatography, magnetic separation, immunoassay, nucleic acid assay, receptor-based assay, cytometric assay, colorimetric assay, enzymatic assay, electrophoretic assay, electrochemical assay, spectroscopic assay, chromatographic assay, microscopic assay, topographic assay, calorimetric assay, radioisotope assay, protein synthesis assay, histological assay, culture assay, and combinations thereof.

Sample preparation can further include dilution by an appropriate solvent and amount to ensure the appropriate range of concentration level is detected by a given assay.

Accessing the nucleic acids and macromolecules from the intercellular space of the sample may generally be performed by either physical, chemical methods, or a combination of both. In some applications of the methods, following the isolation of the crude extract, it will often be desirable to separate the nucleic acids, proteins, cell membrane particles, and the like. In some applications of the methods it will be desirable to keep the nucleic acids with its proteins, and cell membrane particles.

In some applications of the methods provided herein, nucleic acids and proteins can be extracted from a biological sample prior to analysis using methods of the disclosure. Extraction can be by means including, but not limited to, the use of detergent lysates, sonication, or vortexing with glass beads.

In some applications, molecules can be isolated using any technique suitable in the art including, but not limited to, techniques using gradient centrifugation (for example, cesium chloride gradients, sucrose gradients, glucose gradients, etc.), centrifugation protocols, boiling, purification kits, and the use of liquid extraction with agent extraction methods such as methods using Trizol or DNAzol.

Samples may be prepared according to standard biological sample preparation depending on the desired detection method. For example for mass spectrometry detection, biological samples obtained from a patient may be centrifuged, filtered, processed by immunoaffinity column, separated into fractions, partially digested, and combinations thereof. Various fractions may be resuspended in appropriate carrier such as buffer or other type of loading solution for detection and analysis, including LCMS loading buffer.

### Biomarker Assessment

The present disclosure provides for methods for measuring one or more biomarker panels in biological samples. Any suitable method can be used to detect one or more of the biomarkers of any of the panels described herein.

Useful analyte capture agents that can be used in practice of any of the methods described herein include but are not limited to antibodies, such as crude serum containing antibodies, purified antibodies, monoclonal antibodies, polyclonal antibodies, synthetic antibodies, antibody fragments (for example, Fab fragments); antibody interacting agents, such as protein A, carbohydrate binding proteins, and other interactants; protein interactants (for example avidin and its derivatives); peptides; and small chemical entities, such as enzyme substrates, cofactors, metal ions/chelates, aptamers, and haptens. Antibodies may be modified or chemically treated to optimize binding to targets or solid surfaces (for example biochips and columns).

Biomarkers can be measured in a biological sample using an immunoassay. Immunoassays can use an antibody that specifically binds to or recognizes an antigen (for example site on a protein or peptide, biomarker target). An immunoassay can include the steps of contacting the biological sample with the antibody and allowing the antibody to form a complex of with the antigen in the sample, washing the sample and detecting the antibody-antigen complex with a detection reagent. Antibodies that recognize the biomarkers may be commercially available. An antibody that recognizes the biomarkers can be generated by known methods of antibody production.

Immunoassays can include indirect assays, wherein, for example, a second, labeled antibody can be used to detect bound marker-specific antibody. Exemplary detectable labels include magnetic beads (for example, DYNABEADS™), fluorescent dyes, radiolabels, enzymes (for example, horseradish peroxide, alkaline phosphatase and others commonly used), and calorimetric labels such as colloidal gold or colored glass or plastic beads. The biomarker in the sample can be measured using a competition or inhibition assay wherein, for example, a monoclonal antibody which binds to a distinct epitope of the marker is incubated simultaneously with the mixture.

The conditions to detect an antigen using an immunoassay can be dependent on the particular antibody used. Also, the incubation time can depend upon the assay format, marker, volume of solution, concentrations and the like. Immunoassays can be carried out at room temperature, although they can be conducted over a range of temperatures, such as from about 0 degrees to about 40 degrees Celsius depending on the antibody used.

There are various types of immunoassay known in the art that as a starting basis can be used to tailor the assay for the detection of the biomarkers of the present disclosure. Useful assays can include, for example, an enzyme immune assay (EIA) such as enzyme-linked immunosorbent assay (ELISA). For example, if an antigen can be bound to a solid support or surface, it can be detected by reacting it with a specific antibody and the antibody can be quantitated by reacting it with either a secondary antibody or by incorporating a label directly into the primary antibody. Alternatively, an antibody can be bound to a solid surface and the antigen added. A second antibody that recognizes a distinct epitope on the antigen can then be added and detected. Such assay can be referred to as a ‘sandwich assay’ and can be used to avoid problems of high background or non-specific reactions. These types of assays can be sensitive and reproducible enough to measure low concentrations of antigens in a biological sample.

Immunoassays can be used to determine presence or absence of a marker in a sample as well as the quantity of a marker in a sample. Methods for measuring the amount of, or presence of, antibody-marker complex include but are not limited to, fluorescence, luminescence, chemiluminescence, absorbance, reflectance, transmittance, birefringence or refractive index (for example, surface plasmon resonance, ellipsometry, a resonant mirror method, a grating coupler waveguide method or interferometry). Such reagents can be used with optical detection methods, such as various forms of microscopy, imaging methods and non-imaging methods. Electrochemical methods can include voltammetry and amperometry methods. Radio frequency methods can include multipolar resonance spectroscopy.

Measurement of biomarkers can employ the use of an antibody. Antibodies that specifically bind to any of the biomarkers described herein can be prepared using standard methods known in the art. For example polyclonal antibodies can be produced by injecting an antigen into a mammal, such as a mouse, rat, rabbit, goat, sheep, or horse for large quantities of antibody. Blood isolated from these animals can contain polyclonal antibodies—multiple antibodies that bind to the same antigen. Alternatively, polyclonal antibodies can be produced by injecting the antigen into chickens for generation of polyclonal antibodies in egg yolk. In addition, antibodies can be made to specifically recognize modified forms for the biomarkers such as a phosphorylated form of the biomarker, for example, they can recognize a tyrosine or a serine after phosphorylation, but not in the absence of phosphate. In this way antibodies can be used to determine the phosphorylation state of a particular biomarker.

Antibodies can be obtained commercially or produced using well-established methods. To obtain antibodies specific for a single epitope of an antigen, antibody-secreting lymphocytes can be isolated from the animal and immortalized by fusing them with a cancer cell line. The fused cells can be referred to as hybridomas, and can continually grow and secrete antibody in culture. Single hybridoma cells are isolated by dilution cloning to generate cell clones that all produce the same antibody; these antibodies can be referred to as monoclonal antibodies.

Polyclonal and monoclonal antibodies can be purified in several ways. For example, one can isolate an antibody using antigen-affinity chromatography which can be couple to bacterial proteins such as Protein A, Protein G, Protein L or the recombinant fusion protein, Protein A/G followed by detection of via UV light at 280 nm absorbance of the eluate fractions to determine which fractions contain the antibody. Protein A/G can bind to all subclasses of human IgG, making it useful for purifying polyclonal or monoclonal IgG antibodies whose subclasses have not been determined. In addition, Protein A/G can bind to IgA, IgE, IgM and (in some cases to a lesser extent) IgD. Protein A/G can bind to all subclasses of mouse IgG but in some cases does not bind mouse IgA, IgM or serum albumin. This feature can allow Protein A/G to be used for purification and detection of mouse monoclonal IgG antibodies, without interference from IgA, IgM and serum albumin.

Antibodies can be derived from different classes or isotypes of molecules such as, for example, IgA, IgA IgD, IgE, IgM and IgG. The IgA can be designed for secretion in the bodily fluids while others, like the IgM are designed to be expressed on the cell surface. The antibody can be an IgG antibody. In some cases, IgG comprises two subunits including two “heavy” chains and two “light” chains. These can be assembled in a symmetrical structure and each IgG can have two identical antigen recognition domains. The antigen recognition domain can be a combination of amino acids from both the heavy and light chains. The molecule can be roughly shaped like a “Y” and the arms/tips of the molecule comprise the antigen-recognizing regions or Fab (fragment, antigen binding) region, while the stem of Fc (Fragment, crystallizable) region is not necessarily involved in recognition and can be fairly constant. The constant region can be identical in all antibodies of the same isotype, but can differ in antibodies of different isotypes.

It is also possible to use an antibody to detect a protein after fractionation by western blotting. Western blotting can be used for the detection and/or measurement of biomarkers.

One or more detection methods can employ flow cytometry. Flow cytometry can be a laser based, biophysical technology that can be used for biomarker detection, quantification (cell counting) and cell isolation. This technology can be used in the diagnosis of health disorders, especially blood cancers. In general, flow cytometry can comprise suspending single cells in a stream of fluid. A beam of light (usually laser light) of a single wavelength can be directed onto the stream of liquid, and the scatter light caused by a passing cell can be detected by an electronic detection apparatus. A flow cytometry methodology useful in one or more methods described herein can include Fluorescence-activated cell sorting (FACS). FACS can use florescent-labeled antibodies to detect antigens on cell of interest. This additional feature of antibody labeling use in FACS can enable simultaneous multiparametric analysis and quantification based upon the specific light scattering and fluorescent characteristics of each cell florescent-labeled cell and it provides physical separation of the population of cells of interest as well as traditional flow cytometry does.

A wide range of fluorophores can be used as labels in flow cytometry. Fluorophores can be typically attached to an antibody that recognizes a target feature on or in the cell. Examples of suitable fluorescent labels include, but are not limited to: fluorescein (FITC), 5,6-carboxymethyl fluorescein, Texas red, nitrobenz-2-oxa-1,3-diazol-4-yl (NBD), and the cyanine dyes Cy3, Cy3.5, Cy5, Cy5.5 and Cy7. Other Fluorescent labels such as Alexa Fluor® dyes, DNA content dye such as DAPI, and Hoechst dyes are well known in the art and can be easily obtained from a variety of commercial sources. Each fluorophore can have a characteristic peak excitation and emission wavelength, and the emission spectra often overlap. The absorption and emission maxima, respectively, for these fluors can be: FITC (490 nm; 520 nm), Cy3 (554 nm; 568 nm), Cy3.5 (581 nm; 588 nm), Cy5 (652 nm: 672 nm), Cy5.5 (682 nm; 703 nm) and Cy7 (755 nm; 778 nm). The fluorescent labels can be obtained from a variety of commercial sources. Quantum dots can be used in place of traditional fluorophores. Other methods that can be used for detecting include isotope labeled antibodies, such as lanthanide isotopes.

In some cases, the immunoassay comprises immunohistochemistry. Immunohistochemistry can be used to detect expression of the claimed biomarkers in a tissue sample. The antibodies can be detected by direct labeling of the antibodies themselves, for example, with radioactive labels, fluorescent labels, hapten labels such as, biotin, or an enzyme such as horse radish peroxidase or alkaline phosphatase. Alternatively, unlabeled primary antibody can be used in conjunction with a labeled secondary antibody, comprising antisera, polyclonal antisera or a monoclonal antibody specific for the primary antibody. Immunohistochemistry protocols are well known in the art and protocols and antibodies are commercially available. Alternatively, one could make an antibody to the biomarkers or modified versions of the biomarker or binding partners as disclosure herein that would be useful for determining the expression levels of in a tissue sample.

In some cases, measurement of biomarkers comprises use of a biochip. Biochips can be used to screen a large number of macromolecules. Biochips can be designed with immobilized nucleic acid molecules, full-length proteins, antibodies, affibodies (small molecules engineered to mimic monoclonal antibodies), aptamers (nucleic acid-based ligands) or chemical compounds. A chip could be designed to detect multiple macromolecule types on one chip. For example, a chip could be designed to detect nucleic acid molecules, proteins and metabolites on one chip. The biochip can be used to and designed to simultaneously analyze a panel biomarker in a single sample, producing a subjects profile for these biomarkers. The use of the biochip allows for the multiple analyses to be performed reducing the overall processing time and the amount of sample required.

Protein microarray can be a particular type of biochip which can be used with the present disclosure. In some cases, the chip comprises a support surface such as a glass slide, nitrocellulose membrane, bead, or microtitre plate, to which an array of capture proteins can be bound in an arrayed format onto a solid surface. Protein array detection methods can give a high signal and a low background. Detection probe molecules, typically labeled with a fluorescent dye, can be added to the array. Any reaction between the probe and the immobilized protein can result in emission of a detectable signal. Such protein microarrays can be rapid, automated, and offer high sensitivity of protein biomarker read-outs for diagnostic tests. However, it would be immediately appreciated to those skilled in the art that there are a variety of detection methods that can be used with this technology. Exemplary microarrays include analytical microarrays (also known as capture arrays), functional protein microarrays (also known as target protein arrays) and reverse phase protein microarray (RPA).

Analytical protein microarrays can be constructed using a library of antibodies, aptamers or affibodies. The array can be probed with a complex protein solution such as a blood, serum or a cell lysate that function by capturing protein molecules they specifically bind to. Analysis of the resulting binding reactions using various detection systems can provide information about expression levels of particular proteins in the sample as well as measurements of binding affinities and specificities. This type of protein microarray can be especially useful in comparing protein expression in different samples. Functional protein microarrays can be constructed by immobilizing large numbers of purified full-length functional proteins or protein domains and can be used to identify protein-protein, protein-DNA, protein-RNA, protein-phospholipid, and protein-small molecule interactions, to assay enzymatic activity and to detect antibodies and demonstrate their specificity. These protein microarray biochips can be used to study the biochemical activities of the entire proteome in a sample.

One or more biomarkers can be measured using reverse phase protein microarray (RPA). Reverse phase protein microarray can be constructed from tissue and cell lysates that can be arrayed onto the microarray and probed with antibodies against the target protein of interest. These antibodies can be detected with chemiluminescent, fluorescent or colorimetric assays. In addition to the protein in the lysate, reference control peptides can be printed on the slides to allow for protein quantification. RPAs allow for the determination of the presence of altered proteins or other agents that may be the result of disease and present in a diseased cell.

One or more biomarkers can be measured using mass spectroscopy (alternatively referred to as mass spectrometry). Mass spectrometry (MS) can refer to an analytical technique that measures the mass-to-charge ratio of charged particles. It can be primarily used for determining the elemental composition of a sample or molecule, and for elucidating the chemical structures of molecules, such as peptides and other chemical compounds. MS works by ionizing chemical compounds to generate charged molecules or molecule fragments and measuring their mass-to-charge ratios MS instruments typically consist of three modules (1) an ion source, which can convert gas phase sample molecules into ions (or, in the case of electrospray ionization, move ions that exist in solution into the gas phase) (2) a mass analyzer, which sorts the ions by their masses by applying electromagnetic fields and (3) detector, which measures the value of an indicator quantity and thus provides data for calculating the abundances of each ion present.

Suitable mass spectrometry methods to be used with the present disclosure include but are not limited to, one or more of electrospray ionization mass spectrometry (ESI-MS), ESI-MS/MS, ESI-MS/(MS)ₙ, matrix-assisted laser desorption ionization time-of-flight mass spectrometry (MALDI-TOF-MS), surface-enhanced laser desorption/ionization time-of-flight mass spectrometry (SELDI-TOF-MS), tandem liquid chromatography-mass spectrometry (LC-MS/MS) mass spectrometry, desorption/ionization on silicon (DIOS), secondary ion mass spectrometry (SIMS), quadrupole time-of-flight (Q-TOF), atmospheric pressure chemical ionization mass spectrometry (APCI-MS), APCI-MS/MS, APCI-(MS), atmospheric pressure photoionization mass spectrometry (APPI-MS), APPI-MS/MS, and APPI-(MS)ₙ, quadrupole mass spectrometry, Fourier transform mass spectrometry (FTMS), and ion trap mass spectrometry, where n can be an integer greater than zero.

LC-MS can be commonly used to resolve the components of a complex mixture. LC-MS method generally involves protease digestion and denaturation (usually involving a protease, such as trypsin and a denaturant such as, urea to denature tertiary structure and iodoacetamide to cap cysteine residues) followed by LC-MS with peptide mass fingerprinting or LC-MS/MS (tandem MS) to derive sequence of individual peptides. LC-MS/MS can be used for proteomic analysis of complex samples where peptide masses may overlap even with a high-resolution mass spectrometer. Samples of complex biological fluids like human serum may be first separated on an SDS-PAGE gel or HPLC-SCX and then run in LC-MS/MS allowing for the identification of over 1000 proteins.

While multiple mass spectrometric approaches can be used with the methods of the disclosure as provided herein, in some applications it may be desired to quantify proteins in biological samples from a selected subset of proteins of interest. One such MS technique that can be used with the present disclosure is Multiple Reaction Monitoring Mass Spectrometry (MRM-MS), or alternatively referred to as Selected Reaction Monitoring Mass Spectrometry (SRM-MS).

The MRM-MS technique can use a triple quadrupole (QQQ) mass spectrometer to select a positively charged ion from the peptide of interest, fragment the positively charged ion and then measure the abundance of a selected positively charged fragment ion. This measurement can be commonly referred to as a transition and/or transition ion. By way of illustrative example only, a peptide fragment comprising the amino acid sequence IAELLSPGSVDPLTR can comprise one or more of the following exemplary transition ion biomarkers provided in Table 2, below.

TABLE 2
Exemplary transition ions for the peptide sequence
IAELLSPGSVDPLTR

| Transition Ion | Amino Acid Sequence |
|---|---|
| b1 | I |
| b2 | IA |
| b3 | IAE |
| b4 | IAEL |
| b5 | IAELL |
| b6 | IAELLS |
| b7 | IAELLSP |
| b8 | IAELLSPG |
| b9 | IAELLSPGS |
| b10 | IAELLSPGSV |
| b11 | IAELLSPGSVD |
| b12 | IAELLSPGSVDP |
| b13 | IAELLSPGSVDPL |
| b14 | IAELLSPGSVDPLT |
| y14 | AELLSPGSVDPLTR |
| y13 | ELLSPGSVDPLTR |
| y12 | LLSPGSVDPLTR |
| y11 | LSPGSVDPLTR |
| y10 | SPGSVDPLTR |
| y9 | PGSVDPLTR |
| y8 | GSVDPLTR |
| y7 | SVDPLTR |
| y6 | VDPLTR |
| y5 | DPLTR |
| y4 | PLTR |
| y3 | LTR |
| y2 | TR |
| y1 | R |

In some applications the MRM-MS can be coupled with High-Pressure Liquid Chromatography (HPLC) and more recently Ultra High-Pressure Liquid Chromatography (UHPLC). In other applications MRM-MS can be coupled with UHPLC with a QQQ mass spectrometer to make the desired LC-MS transition measurements for all of the peptides and proteins of interest.

In some applications the utilization of a quadrupole time-of-flight (qTOF) mass spectrometer, time-of-flight time-of-flight (TOF-TOF) mass spectrometer, Orbitrap mass spectrometer, quadrupole Orbitrap mass spectrometer or any Quadrupolar Ion Trap mass spectrometer can be used to select for a positively charged ion from one or more peptides of interest. The fragmented, positively charged ions can then be measured to determine the abundance of a positively charged ion for the quantitation of the peptide or protein of interest.

In some applications the utilization of a time-of-flight (TOF), quadrupole time-of-flight (qTOF) mass spectrometer, time-of-flight time-of-flight (TOF-TOF) mass spectrometer, Orbitrap mass spectrometer or quadrupole Orbitrap mass spectrometer can be used to measure the mass and abundance of a positively charged peptide ion from the protein of interest without fragmentation for quantitation. In this application, the accuracy of the analyte mass measurement can be used as selection criteria of the assay. An isotopically labeled internal standard of a known composition and concentration can be used as part of the mass spectrometric quantitation methodology.

In some applications, time-of-flight (TOF), quadrupole time-of-flight (qTOF) mass spectrometer, time-of-flight time-of-flight (TOF-TOF) mass spectrometer, Orbitrap mass spectrometer or quadrupole Orbitrap mass spectrometer can be used to measure the mass and abundance of a protein of interest for quantitation. In this application, the accuracy of the analyte mass measurement can be used as selection criteria of the assay. Optionally this application can use proteolytic digestion of the protein prior to analysis by mass spectrometry. An isotopically labeled internal standard of a known composition and concentration can be used as part of the mass spectrometric quantitation methodology.

In some applications, various ionization techniques can be coupled to the mass spectrometers provide herein to generate the desired information. Non-limiting exemplary ionization techniques that can be used with the present disclosure include but are not limited to Matrix Assisted Laser Desorption Ionization (MALDI), Desorption Electrospray Ionization (DESI), Direct Assisted Real Time (DART), Surface Assisted Laser Desorption Ionization (SALDI), or Electrospray Ionization (ESI).

In some applications, HPLC and UHPLC can be coupled to a mass spectrometer a number of other peptide and protein separation techniques can be performed prior to mass spectrometric analysis. Some exemplary separation techniques which can be used for separation of the desired analyte (for example, peptide or protein) from the matrix background include but are not limited to Reverse Phase Liquid Chromatography (RP-LC) of proteins or peptides, offline Liquid Chromatography (LC) prior to MALDI, 1 dimensional gel separation, 2-dimensional gel separation, Strong Cation Exchange (SCX) chromatography, Strong Anion Exchange (SAX) chromatography, Weak Cation Exchange (WCX), and Weak Anion Exchange (WAX). One or more of the above techniques can be used prior to mass spectrometric analysis.

One or more biomarkers can be measured using a microarray. Differential gene expression can also be identified, or confirmed using the microarray technique. Thus, the expression profile biomarkers can be measured in either fresh or fixed tissue, using microarray technology. In this method, polynucleotide sequences of interest (including cDNAs and oligonucleotides) can be plated, or arrayed, on a microchip substrate. The arrayed sequences can be then hybridized with specific DNA probes from cells or tissues of interest. The source of mRNA can be total RNA isolated from a biological sample, and corresponding normal tissues or cell lines may be used to determine differential expression.

PCR amplified inserts of cDNA clones can be applied to a substrate in a dense array. Preferably at least 10,000 nucleotide sequences can be applied to the substrate. The microarrayed genes, immobilized on the microchip at 10,000 elements each, can be suitable for hybridization under stringent conditions. Fluorescently labeled cDNA probes may be generated through incorporation of fluorescent nucleotides by reverse transcription of RNA extracted from tissues of interest. Labeled cDNA probes applied to the chip hybridize with specificity to each spot of DNA on the array. After stringent washing to remove non-specifically bound probes, the microarray chip can be scanned by a device such as, confocal laser microscopy or by another detection method, such as a CCD camera. Quantitation of hybridization of each arrayed element allows for assessment of corresponding mRNA abundance. With dual color fluorescence, separately labeled cDNA probes generated from two sources of RNA can be hybridized pair-wise to the array. The relative abundance of the transcripts from the two sources corresponding to each specified gene can be thus determined simultaneously. Microarray analysis can be performed by commercially available equipment, following manufacturer's protocols.

One or more biomarkers can be measured using qRT-PCR, which can be used to compare mRNA levels in different sample populations, in normal and tumor tissues, with or without drug treatment, to characterize patterns of gene expression, to discriminate between closely related mRNAs, and to analyze RNA structure. The first step in gene expression profiling by RT-PCR can be extracting RNA from a biological sample followed by the reverse transcription of the RNA template into cDNA and amplification by a PCR reaction. The reverse transcription reaction step can be generally primed using specific primers, random hexamers, or oligo-dT primers, depending on the goal of expression profiling. Reverse transcriptases can be avilo myeloblastosis virus reverse transcriptase (AMV-RT) and/or Moloney murine leukemia virus reverse transcriptase (MLV-RT).

Although the PCR step can use a variety of thermostable DNA-dependent DNA polymerases, it typically employs the Taq DNA polymerase, which can have a 5′-3′ nuclease activity but lacks a 3′-5′ proofreading endonuclease activity. Thus, TaqMan™ PCR typically utilizes the 5′-nuclease activity of Taq or Tth polymerase to hydrolyze a hybridization probe bound to its target amplicon, but any enzyme with equivalent 5′ nuclease activity can be used. Two oligonucleotide primers can be used to generate an amplicon typical of a PCR reaction. A third oligonucleotide, or probe, can be designed to detect nucleotide sequence located between the two PCR primers. The probe can be non-extendible by Taq DNA polymerase enzyme, and can be labeled with a reporter fluorescent dye and a quencher fluorescent dye. Any laser-induced emission from the reporter dye can be quenched by the quenching dye when the two dyes are located close together as they are on the probe. During the amplification reaction, the Taq DNA polymerase enzyme can cleave the probe in a template-dependent manner. The resultant probe fragments can disassociate in solution, and signal from the released reporter dye can be freed from the quenching effect of the second fluorophore. One molecule of reporter dye can be liberated for each new molecule synthesized, and detection of the unquenched reporter dye can provide basis for quantitative interpretation of the data.

TaqMan™ RT-PCR can be performed using commercially available equipment, such as, for example, ABI PRISM 7700™ Sequence Detection System™ (Perkin-Elmer-Applied Biosystems, Foster City, Calif., USA), or Lightcycler (Roche Molecular Biochemicals, Mannheim, Germany). In a preferred embodiment, the 5′ nuclease procedure is run on a real-time quantitative PCR device such as the ABI PRISM 7700™ Sequence Detection System™. The system comprises a thermocycler, laser, charge-coupled device (CCD), camera and computer. The system includes software for running the instrument and for analyzing the data. 5′-Nuclease assay data are initially expressed as Ct, or the threshold cycle. As discussed above, fluorescence values are recorded during every cycle and represent the amount of product amplified to that point in the amplification reaction. The point when the fluorescent signal is first recorded as statistically significant can be the threshold cycle (Ct).

To minimize errors and the effect of sample-to-sample variation, RT-PCR can be performed using an internal standard. An internal standard can be expressed at a constant level among different tissues, and can be unaffected by the experimental treatment. RNAs most frequently used to normalize patterns of gene expression are mRNAs for the housekeeping genes glyceraldehyde-3-phosphate-dehydrogenase (GAPDH) and Beta-Actin.

A more recent variation of the RT-PCR technique can include the real time quantitative PCR, which can measure PCR product accumulation through a dual-labeled fluorogenic probe (i.e., TaqMan™ probe). Real time PCR can be compatible both with quantitative competitive PCR, where internal competitor for each target sequence can be used for normalization, and with quantitative comparative PCR using a normalization gene contained within the sample, or a housekeeping gene for RT-PCR. For further details see, for example Held et al., Genome Research 6:986-994 (1996).

### Normalization of Data

Measurement data used in the methods disclosed herein can be normalized. Normalization can refer to a process to correct for example, differences in the amount of genes or protein levels assayed and variability in the quality of the template used, to remove unwanted sources of systematic variation measurements involved in the processing and detection of genes or protein expression. Other sources of systematic variation are attributable to laboratory processing conditions.

In some instances, normalization methods can be used for the normalization of laboratory processing conditions. Non-limiting examples of normalization of laboratory processing that may be used with methods of the disclosure include but are not limited to: accounting for systematic differences between the instruments, reagents, and equipment used during the data generation process, and/or the date and time or lapse of time in the data collection.

Assays can provide for normalization by incorporating the expression of certain normalizing standard genes or proteins, which do not significantly differ in expression levels under the relevant conditions, that is to say they are known to have a stabilized and consistent expression level in that particular sample type. Suitable normalization genes and proteins that can be used with the present disclosure include housekeeping genes. (See, for example, E. Eisenberg, et al., Trends in Genetics 19(7):362-365 (2003). In some applications, the normalizing biomarkers (genes and proteins), also referred to as reference genes, known not to exhibit meaningfully different expression levels in subjects with advanced colorectal adenoma or CRC as compared to control subjects without advanced colorectal adenoma or CRC. In some applications, it may be useful to add a stable isotope labeled standards which can be used and represent an entity with known properties for use in data normalization. In other applications, a standard, fixed sample can be measured with each analytical batch to account for instrument and day-to-day measurement variability.

In some applications, diagnostic, prognostic and predictive genes may be normalized relative to the mean of at least 2, 3, 4, 5, 6, 7, 8, 9, 10, 15, 20, 25, 30, 40, or 50 or more reference genes and proteins. Normalization can be based on the mean or median signal of all of the assayed biomarkers or by a global biomarker normalization approach. Those skilled in the art will recognize that normalization may be achieved in numerous ways, and the techniques described above are intended only to be exemplary.

### Standardization of Data

Measurement data used in the methods disclosed herein can be standardized. Standardization can refer to a process to effectively put all the genes on a comparable scale. Standardization can be performed, for example, by dividing each expression value by its standard deviation across all samples for that gene or protein.

### Clinical Outcome Score

Machine learning algorithms for sub-selecting discriminating biomarkers and optionally subject characteristics, and for building classification models, can be used to determine clinical outcome scores. These algorithms include, but are not limited to, elastic networks, random forests, support vector machines, and logistic regression. These algorithms can aid in selection of important biomarker features and transform the underlying measurements into a score or probability relating to, for example, clinical outcome, disease risk, disease likelihood, presence or absence of disease, treatment response, and/or classification of disease status.

A clinical outcome score can be determined by comparing a level of at least two biomarkers in the biological sample obtained from the subject to a reference level of the at least two biomarkers. A clinical outcome score can be determined by comparing a subject-specific profile of a biomarker panel to a reference profile of the biomarker panel. In some cases, a reference level or reference profile can represent a known diagnosis. For example, a reference level or reference profile can represent a positive diagnosis of advanced colorectal adenoma. A reference level or reference profile can represent a positive diagnosis of CRC. For other example, a reference level or reference profile can represent a negative diagnosis of advanced colorectal adenoma. A reference level or reference profile can represent a negative diagnosis of CRC

In some embodiments, an increase in a score indicates an increased likelihood of one or more of: a poor clinical outcome, good clinical outcome, high risk of disease, low risk of disease, complete response, partial response, stable disease, non-response, and recommended treatments for disease management. In some embodiments, a decrease in the quantitative score indicates an increased likelihood of one or more of: a poor clinical outcome, good clinical outcome, high risk of disease, low risk of disease, complete response, partial response, stable disease, non-response, and recommended treatments for disease management.

In some embodiments, a similar biomarker profile from a patient to a reference profile indicates an increased likelihood of one or more of: a poor clinical outcome, good clinical outcome, high risk of disease, low risk of disease, complete response, partial response, stable disease, non-response, and recommended treatments for disease management. In some applications, a dissimilar biomarker profile from a patient to a reference profile indicates one or more of: an increased likelihood of a poor clinical outcome, good clinical outcome, high risk of disease, low risk of disease, complete response, partial response, stable disease, non-response, and recommended treatments for disease management.

In some applications, an increase in one or more biomarker threshold values indicates an increased likelihood of one or more of: a poor clinical outcome, good clinical outcome, high risk of disease, low risk of disease, complete response, partial response, stable disease, non-response, and recommended treatments for disease management. In some applications, a decrease in one or more biomarker threshold values indicates an increased likelihood of one or more of: a poor clinical outcome, good clinical outcome, high risk of disease, low risk of disease, complete response, partial response, stable disease, non-response, and recommended treatments for disease management.

In some applications, an increase in at least one of a quantitative score, one or more biomarker thresholds, a similar biomarker profile values indicates an increased likelihood of one or more of: a poor clinical outcome, good clinical outcome, high risk of disease, low risk of disease, complete response, partial response, stable disease, non-response, and recommended treatments for disease management. In some applications, a decrease in at least one of a quantitative score, one or more biomarker thresholds, a similar biomarker profile values or combinations thereof indicates an increased likelihood of one or more of: a poor clinical outcome, good clinical outcome, high risk of disease, low risk of disease, complete response, partial response, stable disease, non-response, and recommended treatments for disease management.

### Computer Systems

Provided herein are computer systems for implementing any of the methods described herein for detecting a presence or absence of at least one of advanced colorectal adenoma and CRC. For example, provided herein are computer systems for detecting a presence or absence of advanced colorectal adenoma. Also provided herein are computer systems for detecting a presence or absence of CRC. Computer systems disclosed herein may comprise a memory unit. The memory unit can be configured to receive data comprising measurement of a biomarker panel from a biological sample of a subject. The biomarker panel can be any biomarker panel described herein. For example, the biomarker panel can comprise at least two biomarkers selected from the group consisting of A1AG1, A1AT, AACT, AMY2B, ANXA1, APOA1, CAH1, CATD, CEAM3, CLUS, CTNB1, CO3, CO9, CRP, CSF1, DPP4, ECH1, FHL1, FIBB, FIBG, FRIL, GELS, HPT, OSTP, PRDX1, SAA1, SBP1, SEPR, SPB6, SPON2, SYG, TIMP1, and TRFE. Optionally, the biomarker panel can consist of: A1AG1, AACT, CO3, CO9, and SAM. Computer systems disclosed herein may comprise computer executable code for performing at least one of: generating a subject-specific profile of a biomarker panel described herein based upon the measurement data, comparing the subject-specific profile of the biomarker panel to a reference profile of the biomarker panel, and determining a likelihood of advanced colorectal adenoma in the subject. Computer systems disclosed herein may comprise computer executable code for performing at least one of: generating a subject-specific profile of a biomarker panel described herein based upon the measurement data, comparing the subject-specific profile of the biomarker panel to a reference profile of the biomarker panel, and determining a likelihood of CRC in the subject.

Additionally, provided herein are computer systems for implementing any of the methods described herein for detecting a presence or absence of at least one of advanced colorectal adenoma and CRC. For example, provided herein are computer systems for detecting a presence or absence of advanced colorectal adenoma. Also provided herein are computer systems for detecting a presence or absence of CRC. Computer systems disclosed herein may comprise a memory unit. The memory unit can be configured to receive data comprising measurement of a biomarker panel from a biological sample of a subject. The biomarker panel can be any biomarker panel described herein. For example, the biomarker panel can comprise at least two biomarkers selected from the group consisting of A1AG1, A1AT, AACT, APOA1, CATD, CEAM3, CLUS, CO3, CO9, CRP, FIBB, FIBG, GELS, OSTP, PRDX1, SAA1, SBP1, and SEPR. Optionally, the biomarker panel can consist of:

A1AG1, A1AT, CATD, CEAM3, CO9, OSTP, and SEPR. Optionally, the biomarker panel can consist of: A1AG1, A1AT, APOA1, CATD, CEAM3, CLUS, CO3, CO9, FIBB, FIBG, GELS, PRDX1, SBP1, and SEPR. Optionally, the biomarker panel can consist of: A1AG1, A1AT, CATD, CEAM3, CO9, and SEPR. Optionally, the biomarker panel can consist of: A1AG1, A1AT, AACT, CATD, CEAM3, CO9, CRP, GELS, SAA1, and SEPR. Optionally, the biomarker panel can consist of: CATD, CEA, CO3, CO9, GELS, and SEPR. Computer systems disclosed herein may comprise computer executable code for performing at least one of: generating a subject-specific profile of a biomarker panel described herein based upon the measurement data, comparing the subject-specific profile of the biomarker panel to a reference profile of the biomarker panel, and determining a likelihood of advanced colorectal adenoma in the subject. Computer systems disclosed herein may comprise computer executable code for performing at least one of: generating a subject-specific profile of a biomarker panel described herein based upon the measurement data, comparing the subject-specific profile of the biomarker panel to a reference profile of the biomarker panel, and determining a likelihood of CRC in the subject.

Computer systems described herein may comprise computer-executable code for performing any of the algorithms described herein. The computer system can further comprise computer-executable code for providing a report communicating the presence or absence of the at least one of advanced colorectal adenoma and CRC, for recommending a colonoscopy, sigmoidoscopy, or colorectal tissue biopsy, and/or for recommending a treatment. In some embodiments, the computer system executes instructions contained in a computer-readable medium.

In some embodiments, the processor is associated with one or more controllers, calculation units, and/or other units of a computer system, or implanted in firmware. In some embodiments, one or more steps of the method are implemented in hardware. In some embodiments, one or more steps of the method are implemented in software. Software routines may be stored in any computer readable memory unit such as flash memory, RAM, ROM, magnetic disk, laser disk, or other storage medium as described herein or known in the art. Software may be communicated to a computing device by any known communication method including, for example, over a communication channel such as a telephone line, the internet, a wireless connection, or by a transportable medium, such as a computer readable disk, flash drive, etc. The one or more steps of the methods described herein may be implemented as various operations, tools, blocks, modules and techniques which, in turn, may be implemented in firmware, hardware, software, or any combination of firmware, hardware, and software. When implemented in hardware, some or all of the blocks, operations, techniques, etc. may be implemented in, for example, an application specific integrated circuit (ASIC), custom integrated circuit (IC), field programmable logic array (FPGA), or programmable logic array (PLA).

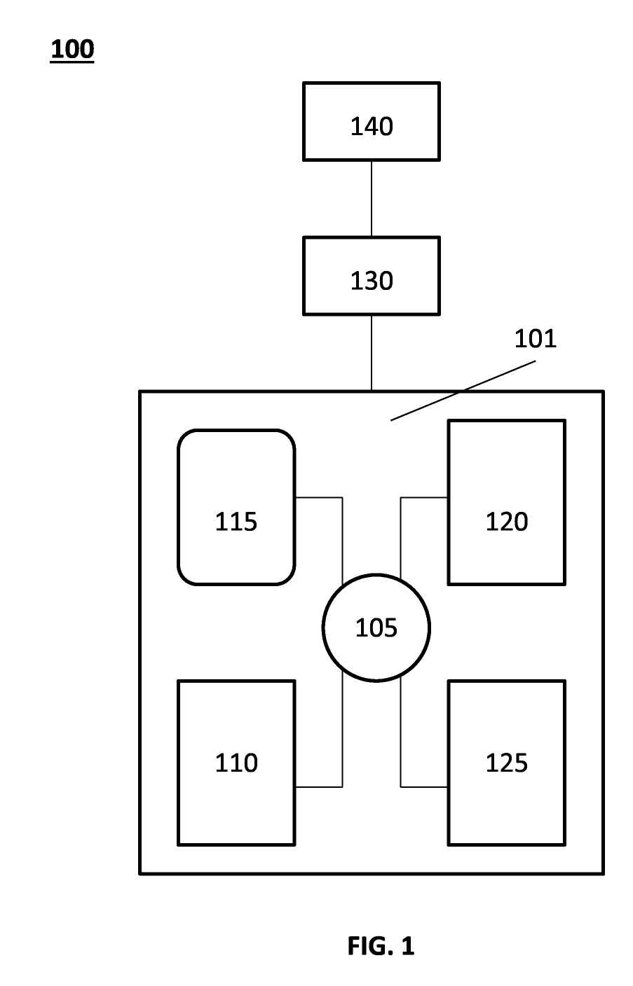

FIG. 1 depicts an exemplary computer system 100 adapted to implement a method described herein. The system 100 includes a central computer server 101 that is programmed to implement exemplary methods described herein. The server 101 includes a central processing unit (CPU, also “processor”) 105 which can be a single core processor, a multi core processor, or plurality of processors for parallel processing. The server 101 also includes memory 110 (for example random access memory, read-only memory, flash memory); electronic storage unit 115 (for example hard disk); communications interface 120 (for example network adaptor) for communicating with one or more other systems; and peripheral devices 125 which may include cache, other memory, data storage, and/or electronic display adaptors. The memory 110, storage unit 115, interface 120, and peripheral devices 125 are in communication with the processor 105 through a communications bus (solid lines), such as a motherboard. The storage unit 115 can be a data storage unit for storing data. The server 101 is operatively coupled to a computer network (“network”) 130 with the aid of the communications interface 120. The network 130 can be the Internet, an intranet and/or an extranet, an intranet and/or extranet that is in communication with the Internet, a telecommunication or data network. The network 130 in some cases, with the aid of the server 101, can implement a peer-to-peer network, which may enable devices coupled to the server 101 to behave as a client or a server.

The storage unit 115 can store files, such as subject reports, and/or communications with the caregiver, sequencing data, data about individuals, or any aspect of data associated with the invention.

The server can communicate with one or more remote computer systems through the network 130. The one or more remote computer systems may be, for example, personal computers, laptops, tablets, telephones, Smart phones, or personal digital assistants.

In some situations the system 100 includes a single server 101. In other situations, the system includes multiple servers in communication with one another through an intranet, extranet and/or the Internet.

The server 101 can be adapted to store measurement data, patient information from the subject, such as, for example, polymorphisms, mutations, medical history, family history, demographic data and/or other information of potential relevance. Such information can be stored on the storage unit 115 or the server 101 and such data can be transmitted through a network.

Methods as described herein can be implemented by way of machine (or computer processor) executable code (or software) stored on an electronic storage location of the server 101, such as, for example, on the memory 110, or electronic storage unit 115. During use, the code can be executed by the processor 105. In some cases, the code can be retrieved from the storage unit 115 and stored on the memory 110 for ready access by the processor 105. In some situations, the electronic storage unit 115 can be precluded, and machine-executable instructions are stored on memory 110. Alternatively, the code can be executed on a second computer system 140.

Aspects of the systems and methods provided herein, such as the server 101, can be embodied in programming. Various aspects of the technology may be thought of as “products” or “articles of manufacture” typically in the form of machine (or processor) executable code and/or associated data that is carried on or embodied in a type of machine readable medium. Machine-executable code can be stored on an electronic storage unit, such memory (for example, read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical, and electromagnetic waves, such as used across physical interfaces between local devices, through wired and optical landline networks and over various air-links. The physical elements that carry such waves, such as wired or wireless likes, optical links, or the like, also may be considered as media bearing the software. As used herein, unless restricted to non-transitory, tangible “storage” media, terms such as computer or machine “readable medium” can refer to any medium that participates in providing instructions to a processor for execution.

Hence, a machine readable medium, such as computer-executable code, may take many forms, including but not limited to, tangible storage medium, a carrier wave medium, or physical transmission medium. Non-volatile storage media can include, for example, optical or magnetic disks, such as any of the storage devices in any computer(s) or the like, such may be used to implement the system. Tangible transmission media can include: coaxial cables, copper wires, and fiber optics (including the wires that comprise a bus within a computer system). Carrier-wave transmission media may take the form of electric or electromagnetic signals, or acoustic or light waves such as those generated during radio frequency (RF) and infrared (IR) data communications. Common forms of computer-readable media therefore include, for example: a floppy disk, a flexible disk, hard disk, magnetic tape, any other magnetic medium, a CD-ROM, DVD, DVD-ROM, any other optical medium, punch cards, paper tame, any other physical storage medium with patterns of holes, a RAM, a ROM, a PROM and EPROM, a FLASH-EPROM, any other memory chip or cartridge, a carrier wave transporting data or instructions, cables, or links transporting such carrier wave, or any other medium from which a computer may read programming code and/or data. Many of these forms of computer readable media may be involved in carrying one or more sequences of one or more instructions to a processor for execution.

The results of detection of a presence or absence of at least one of an advanced colorectal adenoma and CRC, generating a subject report, and/or communicating the report to a caregiver can be presented to a user with the aid of a user interface, such as a graphical user interface.

A computer system may be used to implement one or more steps of a method described herein, including, for example, sample collection, sample processing, measurement of an amount of one or more proteins described herein to produce measurement data, determination of a ratio of a protein to another protein to produce measurement data, comparing measurement data to a reference amount, generating a subject-specific profile of a biomarker panel, comparing the subject-specific profile to a reference profile, receiving medical history, receiving medical records, receiving and storing measurement data obtained by one or more methods described herein, analyzing said measurement data to determine a presence or absence of at least one of an advanced colorectal adenoma and CRC (for example, by performing an algorithm described herein), generating a report, and reporting results to a receiver.

A client-server and/or relational database architecture can be used in any of the methods described herein. In general, a client-server architecture is a network architecture in which each computer or process on the network is either a client or a server. Server computers can be powerful computers dedicated to managing disk drives (file servers), printers (print servers), or network traffic (network servers). Client computers can include PCs (personal computers) or workstations on which users run applications, as well as example output devices as disclosed herein. Client computers can rely on server computers for resources, such as files, devices, and even processing power. The server computer handles all of the database functionality. The client computer can have software that handles front-end data management and receive data input from users.

After performing a calculation, a processor can provide the output, such as from a calculation, back to, for example, the input device or storage unit, to another storage unit of the same or different computer system, or to an output device. Output from the processor can be displayed by a data display, for example, a display screen (for example, a monitor or a screen on a digital device), a print-out, a data signal (for example, a packet), a graphical user interface (for example, a webpage), an alarm (for example, a flashing light or a sound), or a combination of any of the above. In an embodiment, an output is transmitted over a network (for example, a wireless network) to an output device. The output device can be used by a user to receive the output from the data-processing computer system. After an output has been received by a user, the user can determine a course of action, or can carry out a course of action, such as a medical treatment when the user is medical personnel. In some embodiments, an output device is the same device as the input device. Example output devices include, but are not limited to, a telephone, a wireless telephone, a mobile phone, a PDA, a flash memory drive, a light source, a sound generator, a fax machine, a computer, a computer monitor, a printer, an iPod, and a webpage. The user station may be in communication with a printer or a display monitor to output the information processed by the server. Such displays, output devices, and user stations can be used to provide an alert to the subject or to a caregiver thereof.

Data relating to the present disclosure can be transmitted over a network or connections for reception and/or review by a receiver. The receiver can be but is not limited to the subject to whom the report pertains; or to a caregiver thereof, for example, a health care provider, manager, other healthcare professional, or other caretaker; a person or entity that performed and/or ordered the genotyping analysis; a genetic counselor. The receiver can also be a local or remote system for storing such reports (for example servers or other systems of a “cloud computing” architecture). In one embodiment, a computer-readable medium includes a medium suitable for transmission of a result of an analysis of a biological sample.

### Kits

The present disclosure also provides kits. In some cases, a kit described herein comprises one or more compositions, reagents, and/or device components for measuring and/or detecting one or more biomarkers described herein. A kit as described herein can further comprise instructions for practicing any of the methods provided herein. The kits can further comprise reagents to enable the detection of biomarker by various assays types such as ELISA assay, immunoassay, protein chip or microarray, DNA/RNA chip or microarray, RT-PCR, nucleic acid sequencing, mass spectrometry, immunohistochemistry, flow cytometry, or high content cell screening. Kits can also comprise a computer readable medium comprising computer executable code for implementing a method described herein.

In some embodiments, a kit provided herein comprises antibodies to the biomarkers described elsewhere in the disclosure. A kit may comprise at least two antibodies that are each reactive against a biomarkers selected from the group consisting of A1AG1, A1AT, AACT, APOA1, CATD, CEAM3, CLUS, CO3, CO9, CRP, FIBB, FIBG, GELS, OSTP, PRDX1, SAA1, SBP1, and SEPR. In some cases, the kit may comprise antibodies that are reactive against A1AG1, A1AT, CATD, CEAM3, CO9, OSTP, and SEPR. In some cases, the kit may comprise antibodies that are reactive against A1AG1, A1AT, APOA1, CATD, CEAM3, CLUS, CO3, CO9, FIBB, FIBG, GELS, PRDX1, SBP1, and SEPR. In some cases, the kit may comprise antibodies that are reactive against A1AG1, A1AT, CATD, CEAM3, CO9, and SEPR. In some cases, the kit may comprise antibodies that are reactive against A1AG1, A1AT, AACT, CATD, CEAM3, CO9, CRP, GELS, SAA1, and SEPR. In some cases, the kit may comprise antibodies that are reactive against CATD, CEA, CO3, CO9, GELS, and SEPR.

In some embodiments, kits described herein include a packaging material. As used herein, the term “packaging material” can refer to a physical structure housing the components of the kit. The packaging material can maintain sterility of the kit components, and can be made of material commonly used for such purposes (for example, paper, corrugated fiber, glass, plastic, foil, ampules, etc.). Kits can also include a buffering agent, a preservative, or a protein/nucleic acid stabilizing agent.

### EXAMPLES

### Example 1: Discovery and Validation of Protein Biomarker Panels for Diagnosis of CRC

Study Design and Patient Sample Collection

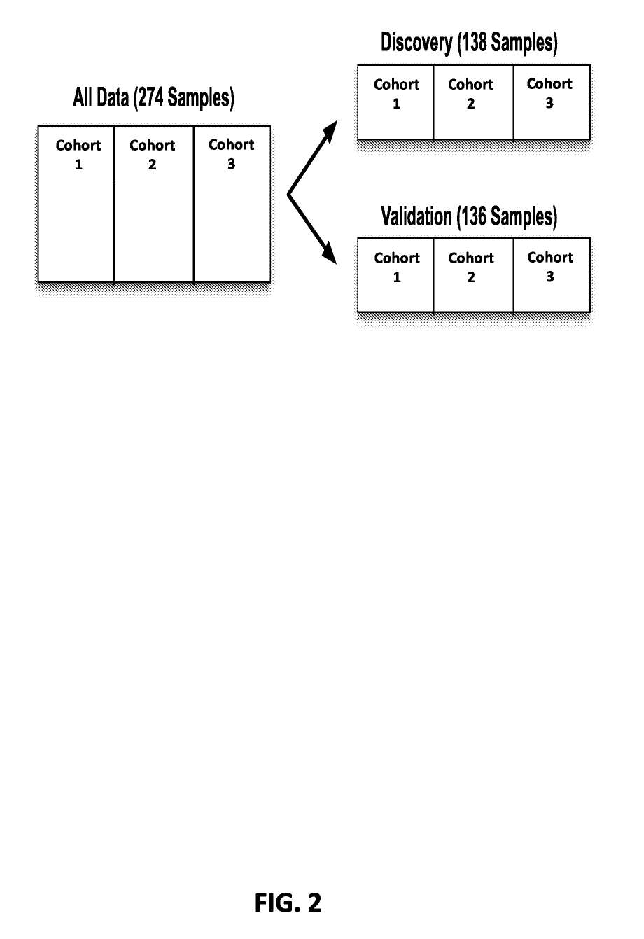

Blood plasma samples from 137 procedure-confirmed CRC and 137 healthy control patients were used in the study. These samples were collected from 3 independent cohorts, and control and disease samples were balanced for age and gender. Approximately half of the control and disease samples (138) were put into a training (for example, discovery) set used for all classifier model development, and the remaining samples (136) were put into a hold-out validation set, which was only used to test the models developed in the discovery set. The discovery and validation sets are depicted in FIG. 2 and in Table 3, below.

TABLE 3
Discovery and training sets for developing protein
biomarker panels for CRC diagnosis.

| | Control | CRC Disease |
|---|---|---|
| Discovery Set | | |
| Cohort 1 | 24 | 24 |
| Cohort 2 | 24 | 24 |
| Cohort 3 | 21 | 21 |
| Total | 69 | 69 |
| Validation Set | | |
| Cohort 1 | 24 | 24 |
| Cohort 2 | 24 | 24 |
| Cohort 3 | 20 | 20 |
| Total | 68 | 68 |

### MRM Assay Development

Initially, 188 proteins previously reported as having association to CRC were interrogated in silico to reveal potential peptide candidates for targeted proteomics profiling. From ten-of-thousands of potential tryptic peptides, a preliminary set of 1056 was selected for experimental verification. A final set of 337 peptides, representing 187 proteins, was selected from experimental verification to comprise the final multiple reaction monitoring (MRM) assay. In addition, 337 complement peptides, of exact sequence composition labeled with heavy (all carbon 13) arginine (R) or lysine (K), were incorporated as internal standards, used in the final analysis as a normalization reference.

Sample Preparation for Plasma Protein Analysis

Patient plasma protein samples were prepared for MRM LCMS measurement according to two methods, referred to as “dilute” and “deplete”, respectively.

In the dilute method, plasma samples were thawed from −80 C storage and lipids and particulates were removed by filter centrifugation. Remaining proteins were reduced to peptides by trypsin-TFE digestion, and the resulting peptides were re-suspended in acetonitrile/formic acid MRM LCMS loading buffer.

In the deplete method, plasma samples were thawed from −80 C storage and lipids and particulates were removed by filter centrifugation. The high-abundance proteins in the filtered plasma were removed by immunoaffinity column-based depletion. The lower abundance, flow-through proteins were reduced to peptides by trypsin-TFE digestion, and the resulting peptides were re-suspended in acetonitrile/formic acid MRM LCMS loading buffer.

For example, patient plasma samples can be prepared for MRM LCMS measurement as follows. Plasma samples were thawed at 4° C. for 30 min followed by a 20-fold dilution of 25 μL of plasma with 475 μL of Multiple-Affinity Removal System (MARS) Buffer A (Agilent). The diluted plasma was filtered through a 0.22 um filter (Agilent), followed by a 5K molecular weight cut-off (MWCO) (Agilent) filtration step for lipid removal. The retentate was reconstituted to 950 μL with MARS Buffer A, and the entire volume was transferred to an autosampler vial for immunoaffinity depletion via a 10 mm×100 mm MARS-14 LC column (Agilent). The flow-through peak of the immunoaffinity column was collected into a 2-mL 96 well plate (Eppendorf). The entire collected sample volume was transferred to a new 5K MWCO filter to exchange the MARS A buffer with 100 mM ammonium bicarbonate prior to a total protein assay (Total Protein Assay, Life Technologies). The sample was transferred to a 2 mL 96-well plate and lyophilized in a proteomic Centrivap system (Labconco). The plate was transferred to a Tecan EV0150 liquid handler for denaturation with 50% 2,2,2-trifluroethanol (TFE) in 100 mM ammonium bicarbonate, reduction with 200 mM DL-dithiothreitol (Sigma), alkylation with 200 mM iodoacetamide (Arcos), and enzymatic digestion with Trypsin (Promega) for 16 hrs at 37° C. The digestion was quenched with 10 L of neat formic acid and transferred to a 330-μL 96-well plate (Costar) for lyophilization. As mentioned below, the LCMS data for the samples were obtained on QQQ mass spectrometers coupled to 1290 UHPLC instruments (Agilent). As an example, a 10 μL injection volume of 3 μg/μL digested plasma was separated on an ZORBAX RRHD Eclipse Plus C18 column (Agilent) with dimensions of 2.1×150 mm, 1.8 um particle size at 450 μL/min. The LC mobile phase A was comprised of 0.1% formic acid in water and mobile phase B was comprised of 0.1% of formic acid in acetonitrile. A 30 minute UHPLC linear segment gradient was used to separate the analytes with the following segments: 3% B for the first 0.5 minutes, 3-6% in 0.5 min, 6-10% in 2 min, 10-30% in 18.75 min, 30-40% in 5 min, 40-80% in 1.25 min, held at 80% for 1.25 min before returning to 3% B in 0.75 min.

Maximizing the number of analytes measured on LCMS in a single injection requires optimizing the amount of time the instrument dwells on any given analyte to a minimum acceptable level while also avoiding overlap or concurrency. An assay was designed to minimize sparse sampling effects due to high frequency in concurrent analytes measured, targeting 12 or more points across a peak for each analyte. The average number of points across the peak was 16.2±5.4. Within the 30 minute chromatography profile, each analyte was allocated a 42 second window for data acquisition with the MS instrument in dynamic MRM (dMRM) acquisition mode. Minimizing the data acquisition window allowed for a maximum single-injection analyte capacity of approximately 1500. Robustness tests for chromatographic drift indicated 150 to 200 LCMS injections could be accomplished without needing to re-adjust targeted retention times or replacing reverse phase LCMS columns. As an example, from 187 selected proteins a total of 1348 transitions were monitored through the dMRM method during each 30 minute LCMS run with a maximum concurrency of 100 transitions. The minimum and maximum dwell times for the dMRM acquisition method were 3.19 and 123.75 milliseconds respectively.

LCMS Data Acquisition and Transition Feature Quantification

Re-suspended peptides from each patient's plasma sample were injected via UHPLC into a triple quadrupole mass spectrometer (QQQ) for quantitative analysis. The collected data (retention time, precursor mass, fragment mass, and ion abundance) were analyzed to detect observed peaks referred to as transitions.

A two-dimensional peak integration algorithm was employed to determine the area under the curve (AUC) for each of the transition peaks.

Complement peptides of exact sequence composition labeled with heavy (all carbon 13) arginine (R) or lysine (K) were utilized as internal standards for each of the 676 targeted transitions. Transition AUC values were normalized with the compliment internal standard AUC value to derive a concentration value for each transition.

Data Normalization, Feature Selection and Classifier Assembly

For classifier assembly and performance evaluation, feature concentration values were used based upon the ratio of the raw peptide peak area to the associated labeled standard peptide raw peak area. For some classification models, ratios of protein features were used where a summary value (for example median, mean, value of transition with maximum signal/noise) of all the transitions associated with a given protein was first computed to obtain per-protein meta features. Next all possible protein ratios were constructed from these protein features to obtain protein ratio features. No normalization of the underlying raw peak areas was applied. Missing values for the transitions were set to the minimum or mean value for each particular transition, or to 0.

Classifier models and the associated classification performance were assessed using a 10 by 10-fold cross validation procedure. A variety of feature selection methods were used; in addition, an exhaustive feature combination search procedure was employed. In the development of the classifier models, only the discovery dataset was used, and was further divided into training and testing sets. In the cross validation procedure, feature selection was first applied to reduce the number of features used, followed by development of the classifier model and subsequent classification performance evaluation. For each of the 10-fold cross validations, the data were segregated into 10 splits each containing 90% of the samples as a training set and the remaining 10% of the samples as a testing set. In this process each of the samples was evaluated one time in a test set. The feature selection and model assembly was performed using the training set only, and these models were then applied to the testing set to evaluate classifier performance. The same procedure was used in the exhaustive feature combination search procedure, except here, no feature selection was performed prior to model development. Instead, all n-choose-r feature combinations were individually evaluated. Because exhaustive enumeration of all feature combinations may not always be computationally feasible, the number of features for n and r were limited to practical values. To obtain the input n features, the maximum information coefficient (MIC) was calculated for all feature pairs, and these pairs were ranked by MIC from high to low. The unique set of the top n features, wherein for the present Example n typically ranges from 50-500, from this ranking were then used in the exhaustive feature set evaluation using values of r, wherein for the present Example r typically ranges from 2 to 6. These features were then evaluated in the 10-fold cross validation procedure described above.

To further assess the generalization of the classification performance, this entire 10-fold cross validation procedure was repeated 10 times, each with a different sampling of training and testing sets.

After this process was completed, the top performing models as assessed by the test set AUC values from the discovery set were selected for validation assessment. The locked-down models were directly applied to the validation data set and the AUC performance was determined.

The total number of transition features used for classifier analysis was 674. To explore the classification performance with fewer numbers of features, Elastic Network regularization was applied as the primary feature selection method prior to building the classification models. In this process, Elastic Network models were built and the model coefficients were used to select the top n features for classification modeling. In another feature selection procedure used for some of the classification models, a combination of 11 different feature selection methods was also used consisting of correlation feature selection, chi-squared filtering, consistency filtering, linear correlation filtering, rank correlation filtering, information gain filtering, ratio gain filtering, symmetrical uncertainty filtering, OneR filtering, random forest filtering and RReliefF filtering. The unique features identified by all 11 methods and the top features ranked by how many of the 11 methods the features were selected in were used for classifier model building. Another feature selection method chose features with the largest difference between control and disease means, among features whose t-test p value was lower than a criterion. The total number of selected features used in the models typically ranged from 2 to 20.

After the feature selection step, a classifier model was built using one of several classification algorithms: the support vector machine (SVM) algorithm, the Random Forest algorithm (RF), Elastic Network regression models, Logistic Regression models, GLMBoost models, k-Nearest Neighbor models, and models based upon single feature univariate AUC performance. After construction of the classifier model on the training set, it was directly applied without modification to the testing set and the associated receiver operator characteristic (ROC) curve was generated. From the generated ROC curves, area under the curve (AUC) was computed. This process resulted in an estimate of the anticipated hold-out set validation performance utilizing only the discovery data. The top performing models from the discovery set evaluation were then applied, without modification, to the validation set to get the actual hold-out set performance. Results from the top 13 performing models are summarized in Table 4, below.

TABLE 4
Summary of top 13 performing models for CRC.

| | Sample | | | | | |
|---|---|---|---|---|---|---|
| | Preparation | | | | Discovery | Validation |
| Model # | Method | Model | # Predictors | Proteins | AUC | AUC |
| 1 | Dilution | GLMBoost | 15 transitions | A1AG1, A1AT, AACT, | 0.82 +− 0.01 | 0.83 |
| | Method | | | ANXA1, APOA1, CO9, | | |
| | | | | CRP, CSF1, FHL1, FIBG, | | |
| | | | | GELS, HPT, SAA1 | | |
| 2 | Dilution | SVM | 2 protein | APOA1, CO3, CO9 | 0.80 +− 0.01 | 0.81 |
| | Method | | ratios | (ratios: APOA1/CO3 and | | |
| | | | | APOA1/CO9) | | |
| 3 | Dilution | SVM | 5 transitions | AACT, CO3, CO9, CRP, | 0.79 +− 0.02 | 0.81 |
| | Method | | | GELS | | |
| 4 | Dilution | SVM | 5 transitions | SPB6, GELS, A1AT, | 0.83 +− 0.06 | 0.79 |
| | Method | | | FIBG, CO3 | | |
| 5 | Depletion | Random | 15 transitions | A1AG1, A1AT, AMY2B, | 0.82 +− 0.01 | 0.91 |
| | Method | Forest | | CLUS, CO9, ECH1, | | |
| | | | | FRIL, GELS, OSTP, | | |
| | | | | SBP1, SEPR, SPON2, | | |
| | | | | TIMP1 | | |
| 6 | Depletion | Elastic | 2 transitions | CO9, GELS | 0.81 +− 0.002 | 0.87 |
| | Method | Network | | | | |
| 7 | Depletion | SVM | 4 transitions | GELS, PRDX1, CO9, | 0.82 +− 0.05 | 0.84 |
| | Method | | | CATD | | |
| 8 | Dilution | SVM | 2 protein | A1AT, APOA1, FIBG | 0.77 +− 0.02 | 0.81 |
| | Method | | ratios | (ratios: A1AT/APOA1 and | | |
| | | | | APOA1/FIBG) | | |
| 9 | Dilution | Univariate | Males only: | A1AT, TRFE | 0.85 | 0.89 |
| | Method | AUC | 1 protein | (ratio: A1AT/TRFE) | | |
| | | | ratio | | | |
| 10 | Dilution | SVM | 4 transitions | APOA1, A1AT, FIBB, | 0.83 +− 0.04 | 0.80 |
| | Method | | | CEAM3 | | |
| 11 | Depletion | SVM | 4 features | CAH1, CRP, FIBG, | 0.79 +− 0.05 | 0.78 |
| | Method | | | CTNB1 | | |
| 12 | Depletion | Elastic | Males only: | CRP, SEPR, SBP1, SRC, | 0.89 +− 0.02 | 0.78 |
| | Method | Network | 5 proteins | DPP4 | | |
| 13 | Depletion | k-Nearest | 3 transitions | CRP, TIMP | 0.78 +− 0.01 | 0.76 |
| | Method | Neighbors | | | | |
| 14 | Depletion | SVM | 3 transitions | SYG, AACT, CO9 | 0.83 +− 0.007 | 0.86 |
| | Method | | | | | |

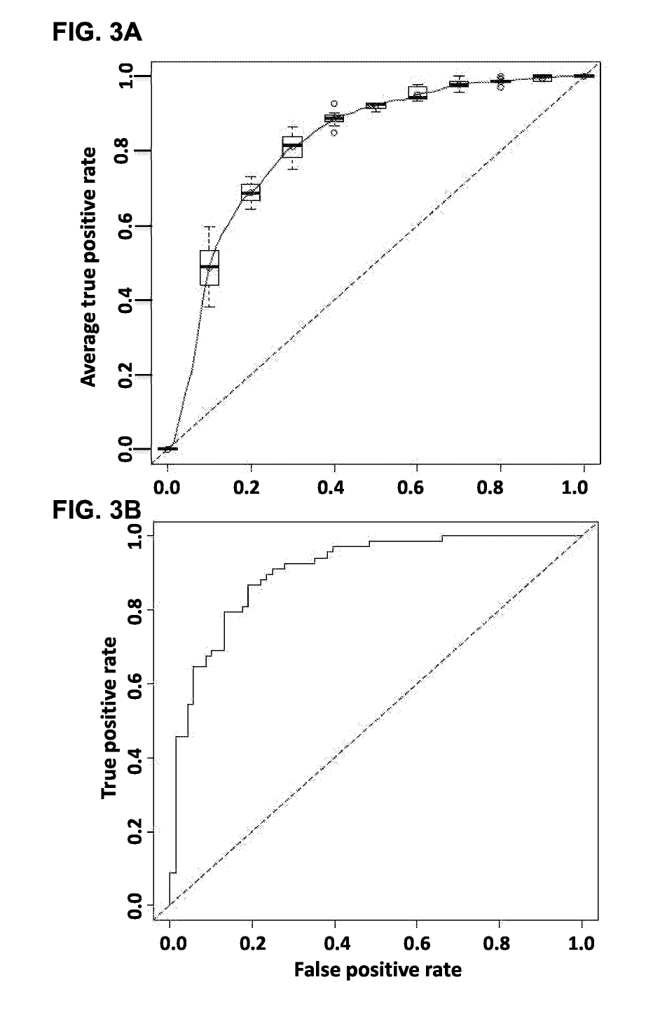

Of the 13 top models, three models provided high classification performance in the discovery and validation datasets. The top performing model was model 5. Model 5 included 15 transition features from 13 proteins that were selected using the combination of the 11 feature selection methods described above with a Random Forest model. The 13 proteins of the first model were A1AG1, A1AT, AMY2B, CLUS, CO9, ECH1, FRIL, GELS, OSTP, SBP1, SEPR, SPON2, TIMP1. ROC curves resulting from the discovery set and the validation set for Model 5 are depicted in FIGS. 3A and 3B, respectively. The resulting discovery set AUC was 0.82+−0.01 (FIG. 3A) and the validation AUC was 0.91 (FIG. 3B). At the validation ROC point of maximum accuracy, the sensitivity was 0.87 and the specificity was 0.81.

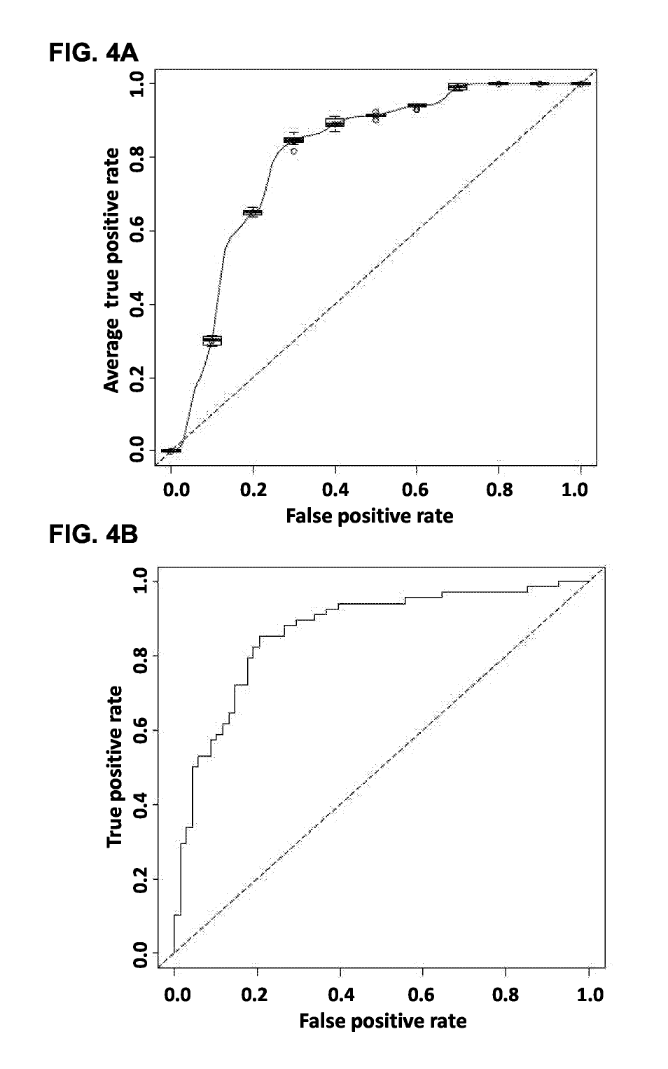

The second top performing model was model 6. Model 6 included two transitions from two proteins which were CO9 and GELS. ROC curves resulting from the discovery set and the validation set for Model 6 are depicted in FIGS. 4A and 4B, respectively. The resulting discovery set AUC was 0.81+/−0.002 (FIG. 4A) and the validation AUC was 0.87 (FIG. 4B). At the validation ROC point of maximum accuracy, the sensitivity was 0.85 and the specificity was 0.79.

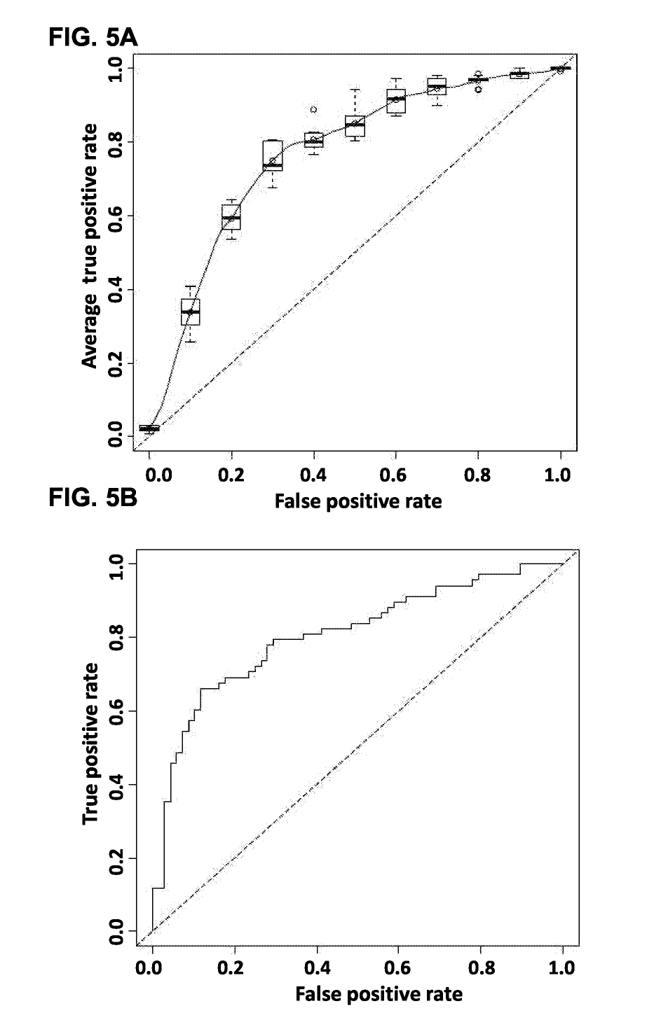

Another top performing model was model 8. Model 8 included two protein ratio features from 3 proteins that were selected using Elastic Network feature selection and an SVM model (linear kernel). The two protein ratios were A1AT/APOA1 and APOA1/FIBG. ROC curves resulting from the discovery set and the validation set for Model 8 are depicted in FIGS. 5A and 5B, respectively. The resulting discovery set AUC was 0.77+/−0.02 (FIG. 5A) and the validation AUC was 0.81 (FIG. 5B). At the validation ROC point of maximum accuracy, the sensitivity was 0.66 and the specificity was 0.88.

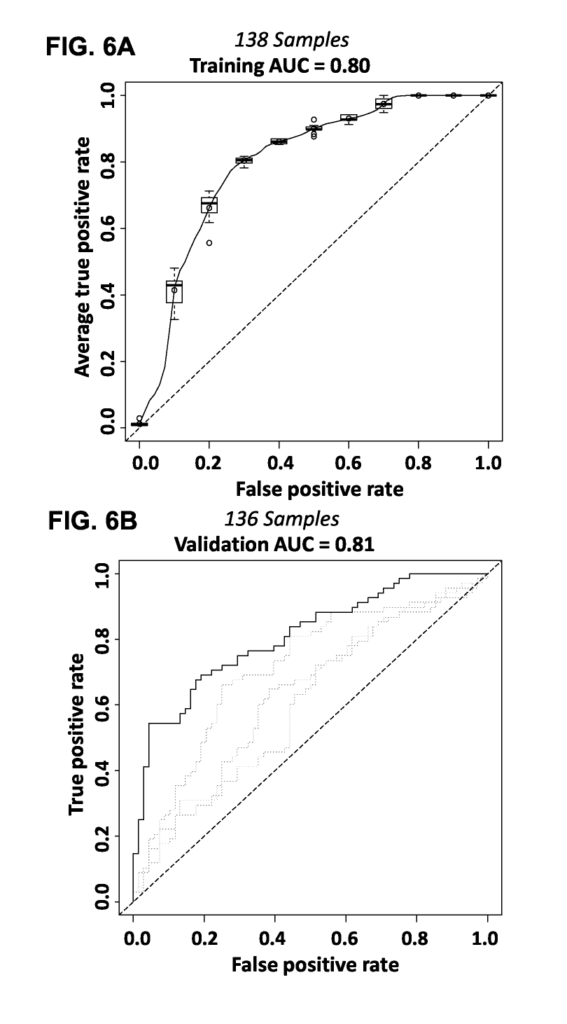

Another top performing model was Model 1, which included 15 transition features from 13 proteins which were A1AG1, A1AT, AACT, ANXA1, APOA1, CO9, CRP, CSF1, FHL1, FIBG, GELS, HPT, SAA1. Another top performing model was Model 2, which included two protein ratio features from 3 proteins which were APOA1, CO3, and CO9. The two protein ratios were APOA1/CO3 and APOA1/CO9. ROC curves generated from Model 2 are depicted in FIG. 6A (ROC curve from discovery set) and FIG. 6B (ROC curve from validation set). Yet another top performing model was Model 7, which included 4 transition features from 4 proteins which were GELS, PRDX1, CO9, and CATD. This model results in a validation AUC of 0.84.

### Example 2: Misclassification Analysis by Sample Set

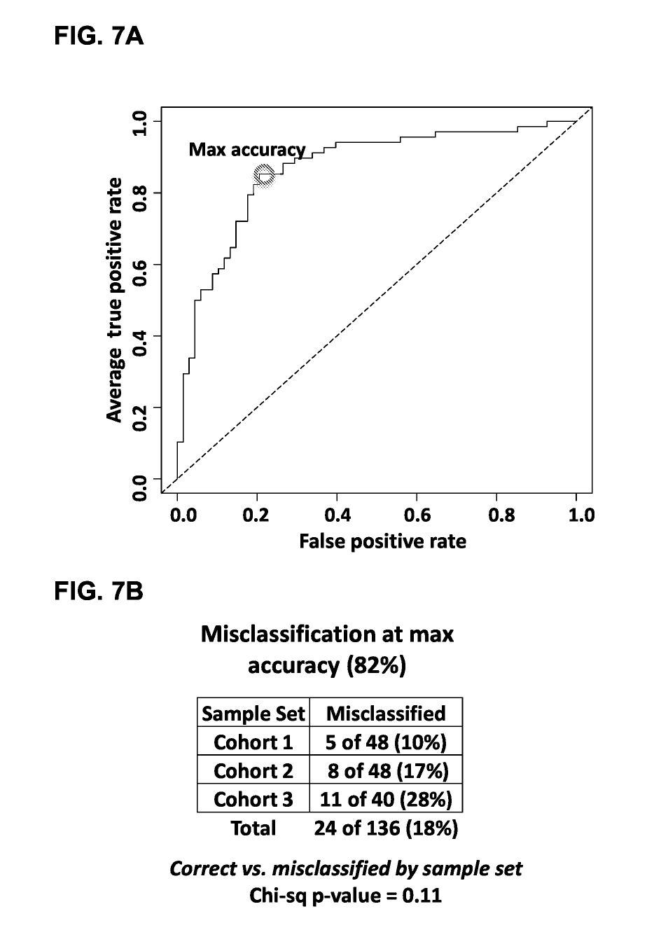

Experiments were conducted to determine whether misclassifications of CRC or no CRC are influenced by the sample set used. Sample sets from cohort 1, cohort 2, and cohort 3, respectively were assessed using Model 6 (measurement of CO9 and GELS). Validation ROC curves from this experiments are shown in FIG. 7A and the point of maximum accuracy (82%) is indicated by the circle. Using the point of maximum accuracy as a diagnosis decision threshold, misclassification of the individual cohort sample sets was assessed. FIG. 7B demonstrates similar misclassification prevalence in all the sample sets with a chi-sq p-value of 0.11, demonstrating that the misclassification of samples is not biased by sample set.

### Example 3: Discovery and Validation of Protein Biomarker Panels Predictive of Advanced Colorectal Adenoma

In order to correlate plasma protein profiles with patient colonoscopy outcomes, blood samples were collected from patients presenting for colonoscopies on the day of their procedures. Inclusion criteria required that the patient be equal to or greater than 18 years of age and be willing and able to sign an informed consent. This was an “all comers” study in which patients could be undergoing the procedure as a recommended, routine screen, as a precaution due to prior personal or family history, or as a follow up to personal health symptoms.

After the routine preparation for colonoscopy that included overnight fasting, liquid-type constraints, and bowel prep to remove fecal matter, a blood sample was drawn into a plasma collection device that included EDTA as an anti-coagulant. The blood sample was mixed, centrifuged to separate plasma as per the manufacturer's instructions, and the separated plasma was collected and frozen at −80 C within four hours.

In addition to the plasma sample, patient clinical data such as age, weight, gender, ethnicity, current medications and indications, and personal and family health history were collected as were the colonoscopy procedure report and the pathology report on any collected and examined tissues. More than 500 patient samples were collected.

136 samples (68 with advanced colorectal adenomas, 68 controls) were selected for classifier analysis. The advanced colorectal adenoma samples were selected from the larger study cohort based upon the size or type of the most severe adenoma. Here, adenomas>=1 cm, or of villous character were classified as advanced colorectal adenomas. The control samples were selected from the larger study cohort by matching age and gender to the advanced colorectal adenoma samples.

MRM Assay Development

MRM assay development was conducted as described in Example 1.

Sample Preparation for Plasma Protein Analysis

Patient plasma protein samples were prepared for MRM LCMS measurement as described in Example 1.

LCMS Data Acquisition and Transition Feature Quantification

LCMS data acquisition and transition feature quantification was performed as described in Example 1.

Data Normalization, Feature Selection and Classifier Assembly

For classifier assembly and performance evaluation, feature concentration values were used based upon the ratio of the raw peptide peak area to the associated labeled standard peptide raw peak area. For some classification models, ratios of protein features were used where a summary value (for example median, mean, value of transition with maximum signal/noise) all the transitions associated with a given protein were first computed to obtain per-protein meta features. Next all possible protein ratios were constructed from these protein features to obtain protein ratio features. No normalization of the underlying raw peak areas was applied. Missing values for the transitions were set to the minimum, mean or median value for each particular transition, or to 0.

Classifier models and the associated classification performance were assessed using a 10 by 10-fold cross validation procedure. A variety of feature selection methods were used; in addition, an exhaustive feature combination search procedure was employed. In the development of the classifier models, the data set was divided into training and testing sets evaluated through a cross validation procedure. In the cross validation procedure, feature selection was first applied to reduce the number of features used, followed by development of the classifier model and subsequent classification performance evaluation. For each of the 10-fold cross validations, the data were segregated into 10 splits each containing 90% of the samples as a training set and the remaining 10% of the samples as a testing set. In this process each of the samples was evaluated one time in a test set. The feature selection and model assembly was performed using the training set only, and these models were then applied to the testing set to evaluate classifier performance. The same procedure was used in the exhaustive feature combination search procedure, except here, no feature selection was performed prior to model development. Instead, all n-choose-r feature combinations were individually evaluated. Because exhaustive enumeration of all feature combinations is not always computationally feasible, the number of features for n and r were limited to practical values. For typical calculations n was, at most, the total number of transition features (674) and r<10. In some calculations, filtering of the transitions based upon feature quality was also employed to reduce the total number of features to evaluate.

To further assess the generalization of the classification performance, this entire 10-fold cross validation procedure was repeated 10 times, each with a different sampling of training and testing sets.

After this process was completed, the top performing models were assessed by the test set AUC values from the cross validation procedure.

The total number of transition features used for classifier analysis was 674. To explore the classification performance with fewer numbers of features, Elastic Network regularization was applied as a feature selection method prior to building the classification models. In this process, Elastic Network models were built and the model coefficients were used to select the top n features for modeling. In another feature selection procedure all possible n-choose-r feature combinations were individually evaluated.

After the feature selection step, a classifier model was built using one of several classification algorithms including, as examples, the support vector machine (SVM) algorithm, the Random Forest algorithm, Elastic Network regression models, and Logistic Regression models. After construction of the classifier model on the training set, it was directly applied without modification to the testing set and the associated receiver operator characteristic (ROC) curve was generated from which the area under the curve (AUC) was computed. This process resulted in an estimate of the anticipated hold-out set validation performance utilizing only the discovery data.

Two models provided high classification performance. The first model comprised 4 transition features from 4 proteins that were identified through an exhaustive search of all 4 feature classifiers. The four proteins for Model 1 and their transition features are shown in Table 5 below.

TABLE 5
Model 1 for advanced colorectal adenoma

| Protein | Transition feature |
|---|---|
| FUCO | DGLIVPIFQER_y6 |
| FIBB | QGFGNVATNTDGK_y6 |
| CATD | QPGITFIAAK_y8 |
| SAHH | VADIGLAAWGR_y7 |

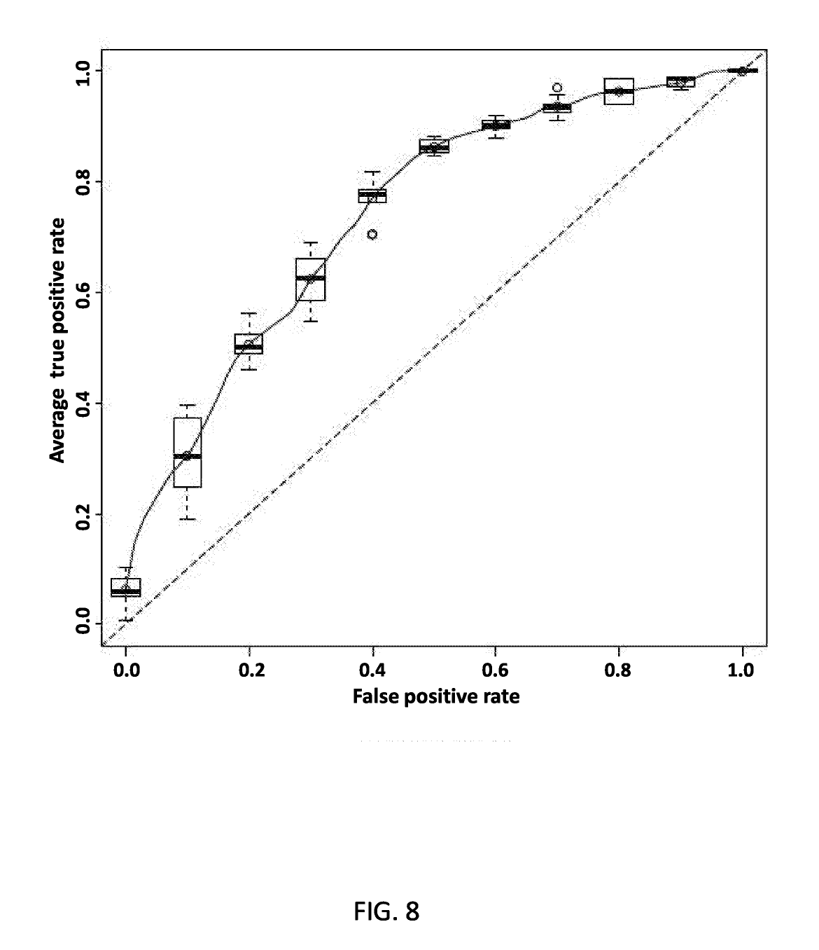

The resulting cross-validation test set AUC for Model 1 (above) was 0.77+−0.01 (FIG. 8).

The second model comprised three transitions from three proteins identified through an exhaustive search of all 3 feature classifiers. The three proteins for Model 2 and their transition features are shown in Table 6 below.

TABLE 6
Model 2 for advanced colorectal adenoma

| Protein | Transition feature |
|---|---|
| CATD | QPGITFIAAK_y8 |
| CATS | GPVSVGVDAR_y6 |
| FUCO | DGLIVPIFQER_y6 |

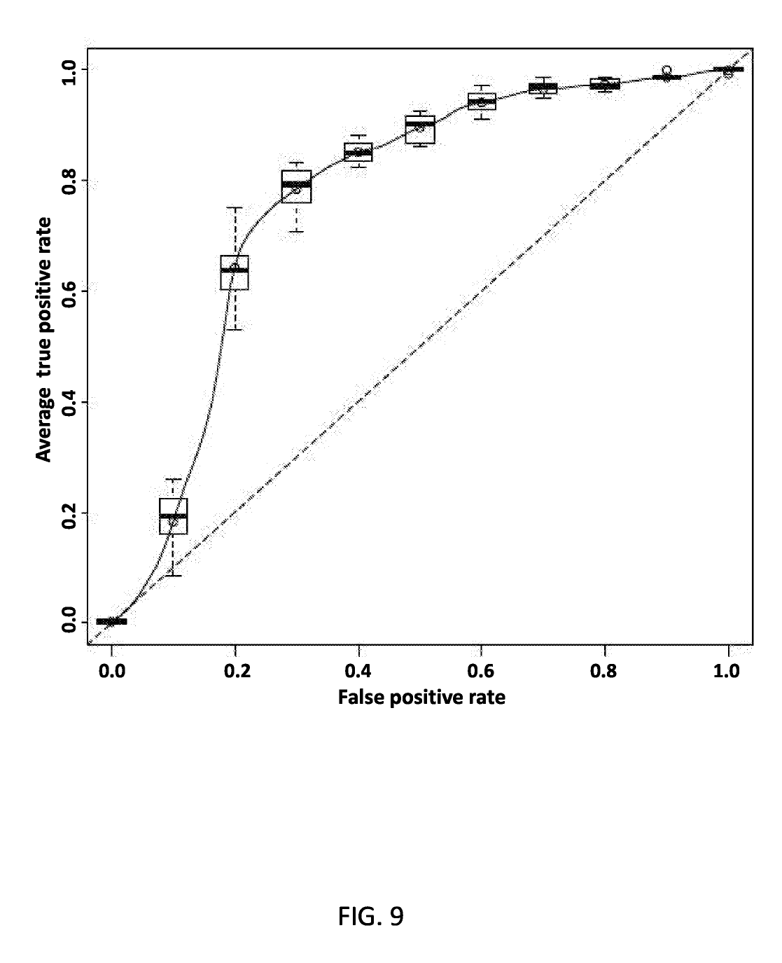

The resulting cross-validation test set AUC for Model 2 (above) gave an AUC of 0.74+−0.02 (FIG. 9).

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

### Example 4

A patient at risk of colorectal cancer is tested using a panel as disclosed herein. A blood sample is taken from the patient and protein accumulation levels are measured for a panel comprising CATD, CEA, CO9 and SEPR, among other markers. The patient's panel results are compared to panel results of known status, and the patient is categorized with an 80% sensitivity and an 80% specificity as having colon cancer.

A colonoscopy is recommended and evidence of colorectal cancer is detected in the individual.

### Example 5

The patient of Example 4 is prescribed a treatment regimen comprising a surgical intervention. A blood sample is taken from the patient prior to surgical intervention and protein accumulation levels are measured for a panel comprising CATD, CEA, CO9 and SEPR, among other markers. The patient's panel results are compared to panel results of known status, and the patient is categorized with an 80% sensitivity and an 80% specificity as having colon cancer.

A blood sample is taken from the patient subsequent to surgical intervention and protein accumulation levels are measured for a panel comprising CATD, CEA, CO9 and SEPR, among other markers. The patient's panel results are compared to panel results of known status, and the patient is categorized with an 80% sensitivity and an 80% specificity as no longer having colon cancer.

### Example 6

The patient of Example 4 is prescribed a treatment regimen comprising a chemotherapeutic intervention comprising 5-FU administration. A blood sample is taken from the patient prior to chemotherapeutic intervention and protein accumulation levels are measured for a panel comprising CATD, CEA, CO9 and SEPR, among other markers. The patient's panel results are compared to panel results of known status, and the patient is categorized with an 80% sensitivity and an 80% specificity as having colon cancer.

A blood sample is taken from the patient at weekly intervals during chemotherapy treatment and protein accumulation levels are measured for a panel comprising CATD, CEA, CO9 and SEPR, among other markers. The patient's panel results are compared to panel results of known status. The patient's panel results over time indicate that the cancer has responded to the chemotherapy treatment and that the colorectal cancer is no longer detectable by completion of the treatment regimen.

### Example 7

The patient of Example 4 is prescribed a treatment regimen comprising a chemotherapeutic intervention comprising oral capecitabine administration. A blood sample is taken from the patient prior to chemotherapeutic intervention and protein accumulation levels are measured for a panel comprising CATD, CEA, CO9 and SEPR, among other markers. The patient's panel results are compared to panel results of known status, and the patient is categorized with an 80% sensitivity and an 80% specificity as having colon cancer.

A blood sample is taken from the patient at weekly intervals during chemotherapy treatment and protein accumulation levels are measured for a panel comprising CATD, CEA, CO9 and SEPR, among other markers. The patient's panel results are compared to panel results of known status. The patient's panel results over time indicate that the cancer has responded to the chemotherapy treatment and that the colorectal cancer is no longer detectable by completion of the treatment regimen.

### Example 8

The patient of Example 4 is prescribed a treatment regimen comprising a chemotherapeutic intervention comprising oral oxaliplatin administration. A blood sample is taken from the patient prior to chemotherapeutic intervention and protein accumulation levels are measured for a panel comprising CATD, CEA, CO9 and SEPR, among other markers. The patient's panel results are compared to panel results of known status, and the patient is categorized with an 80% sensitivity and an 80% specificity as having colon cancer.

A blood sample is taken from the patient at weekly intervals during chemotherapy treatment and protein accumulation levels are measured for a panel comprising CATD, CEA, CO9 and SEPR, among other markers. The patient's panel results are compared to panel results of known status. The patient's panel results over time indicate that the cancer has responded to the chemotherapy treatment and that the colorectal cancer is no longer detectable by completion of the treatment regimen.

### Example 9

The patient of Example 4 is prescribed a treatment regimen comprising a chemotherapeutic intervention comprising oral oxaliplatin administration in combination with bevacizumab. A blood sample is taken from the patient prior to chemotherapeutic intervention and protein accumulation levels are measured for a panel comprising CATD, CEA, CO9 and SEPR, among other markers. The patient's panel results are compared to panel results of known status, and the patient is categorized with an 80% sensitivity and an 80% specificity as having colon cancer.

A blood sample is taken from the patient at weekly intervals during chemotherapy treatment and protein accumulation levels are measured for a panel comprising CATD, CEA, CO9 and SEPR, among other markers. The patient's panel results are compared to panel results of known status. The patient's panel results over time indicate that the cancer has responded to the chemotherapy treatment and that the colorectal cancer is no longer detectable by completion of the treatment regimen.

### Example 10

A patient at risk of colorectal cancer is tested using a panel as disclosed herein. A blood sample is taken from the patient and protein accumulation levels are measured using reagents in an ELISA kit to detect members of a panel comprising CATD, CEA, CO9 and SEPR, among other markers. The patient's panel results are compared to panel results of known status, and the patient is categorized with an 80% sensitivity and an 80% specificity as having colon cancer.

A colonoscopy is recommended and evidence of colorectal cancer is detected in the individual.

### Example 11

A patient at risk of colorectal cancer is tested using a panel as disclosed herein. A blood sample is taken from the patient and protein accumulation levels are measured using mass spectrometry to detect members of a panel comprising CATD, CEA, CO9 and SEPR, among other markers. The patient's panel results are compared to panel results of known status, and the patient is categorized with an 80% sensitivity and an 80% specificity as having colon cancer.

A colonoscopy is recommended and evidence of colorectal cancer is detected in the individual.

### Example 12

1000 patients at risk of colorectal cancer is tested using a panel as disclosed herein. A blood sample is taken from the patient and protein accumulation levels are measured to detect members of a panel comprising CATD, CEA, CO9 and SEPR, among other markers. The patients' panel results are compared to panel results of known status, and the patients are categorized with an 80% sensitivity and an 80% specificity into a colon cancer category.

A colonoscopy is recommended for patients categorized as positive. Of the patients categorized as having colon cancer, 80% are independently confirmed to have colon cancer.

Of the patients categorized as not having colon cancer, 20% are later found to have colon cancer through an independent follow up test, confirmed via a colonoscopy.

What is claimed is:

1. A method of categorizing a colorectal cancer status of an individual, comprising the steps of:
obtaining a blood sample from the individual;
determining protein accumulation levels for a panel of proteins in the blood sample;
comparing protein accumulation levels of a panel of proteins in a sample obtained from said individual to levels corresponding to a known cancer status, and
categorizing said colorectal cancer status of said individual as said known colorectal cancer status if said individual's colorectal cancer status panel protein accumulation levels substantially match said colorectal cancer status panel protein accumulation levels corresponding to said known colorectal cancer status,
wherein said categorizing has a specificity of at least 70%, and wherein said categorizing has a specificity of at least 70%.

2. The method of claim 1, wherein said categorizing has a specificity of at least 75%.

3. The method of claim 1, wherein said categorizing has a specificity of at least 80%.

4. The method of claim 1, wherein said categorizing has a specificity of at least 85%.

5. The method of claim 1, wherein said categorizing has a specificity of at least 90%.

6. The method of claim 1, wherein said categorizing has a sensitivity of at least 75%.

7. The method of claim 1, wherein said categorizing has a sensitivity of at least 80%.

8. The method of claim 1, wherein said categorizing has a sensitivity of at least 85%.

9. The method of claim 1, wherein said categorizing has a sensitivity of at least 90%.

10. The method of claim 1, wherein said determining comprises subjecting said blood sample to an ELISA assay.

11. The method of claim 1, wherein said determining comprises subjecting said blood sample to a mass spectrometric assay.

12. The method of any one of claims 1-11, wherein the panel comprises A1AG1, A1AT, CATD, CEA, CO9, OSTP, and SEPR.

13. The method of any one of claims 1-11, wherein the panel comprises A1AG1, A1AT, APOA1, CATD, CEA, CLUS, CO3, CO9, FGB, FIBG, GELS, PRDX1, SBP1, AND SEPR.

14. The method of any one of claims 1-11, wherein the panel comprises A1AG1, A1AT, CATD, CEA, CO9, and SEPR.

15. The method of any one of claims 1-11, wherein the panel comprises A1AG1, A1AT, AACT, CATD, CEA, CO9, CRP, GELS, SAA1, and SEPR.

16. The method of any one of claims 1-11, wherein the panel comprises CATD, CEA, CO3, CO9, GELS, and SEPR.

17. The method of any one of claims 1-11, wherein the panel comprises at least two proteins selected from the group consisting of A1AG1, A1AT, AACT, AMY2B, ANXA1, APOA1, CAH1, CATD, CEAM3, CLUS, CO3, CO9, CRP, CSF1, CTNB1, DPP4, ECH1, FHL1, FIBB, FIBG, FRIL, GELS, HPT, OSTP, PRDX1, SAA1, SBP1, SEPR, SPB6, SPON2, SRC, SYG, TIMP, TIMP1, and TRFE.

18. The method of any one of claims 1-11, wherein the panel comprises at least three proteins selected from the group consisting of A1AG1, A1AT, AACT, AMY2B, ANXA1, APOA1, CAH1, CATD, CEAM3, CLUS, CO3, CO9, CRP, CSF1, CTNB1, DPP4, ECH1, FHL1, FIBB, FIBG, FRIL, GELS, HPT, OSTP, PRDX1, SAA1, SBP1, SEPR, SPB6, SPON2, SRC, SYG, TIMP, TIMP1, and TRFE.

19. The method of any one of claims 1-11, wherein the panel comprises at least four proteins selected from the group consisting of A1AG1, A1AT, AACT, AMY2B, ANXA1, APOA1, CAH1, CATD, CEAM3, CLUS, CO3, CO9, CRP, CSF1, CTNB1, DPP4, ECH1, FHL1, FIBB, FIBG, FRIL, GELS, HPT, OSTP, PRDX1, SAA1, SBP1, SEPR, SPB6, SPON2, SRC, SYG, TIMP, TIMP1, and TRFE.

20. The method of any one of claims 1-11, wherein the panel comprises at least five proteins selected from the group consisting of A1AG1, A1AT, AACT, AMY2B, ANXA1, APOA1, CAH1, CATD, CEAM3, CLUS, CO3, CO9, CRP, CSF1, CTNB1, DPP4, ECH1, FHL1, FIBB, FIBG, FRIL, GELS, HPT, OSTP, PRDX1, SAA1, SBP1, SEPR, SPB6, SPON2, SRC, SYG, TIMP, TIMP1, and TRFE.

21. The method of any one of claims 1-11, wherein the panel comprises at least six proteins selected from the group consisting of A1AG1, A1AT, AACT, AMY2B, ANXA1, APOA1, CAH1, CATD, CEAM3, CLUS, CO3, CO9, CRP, CSF1, CTNB1, DPP4, ECH1, FHL1, FIBB, FIBG, FRIL, GELS, HPT, OSTP, PRDX1, SAA1, SBP1, SEPR, SPB6, SPON2, SRC, SYG, TIMP, TIMP1, and TRFE.

22. The method of any one of claims 1-11, wherein the panel comprises at least seven proteins selected from the group consisting of A1AG1, A1AT, AACT, AMY2B, ANXA1, APOA1, CAH1, CATD, CEAM3, CLUS, CO3, CO9, CRP, CSF1, CTNB1, DPP4, ECH1, FHL1, FIBB, FIBG, FRIL, GELS, HPT, OSTP, PRDX1, SAA1, SBP1, SEPR, SPB6, SPON2, SRC, SYG, TIMP, TIMP1, and TRFE.

23. The method of any one of claims 1-11, wherein the panel comprises at least eight proteins selected from the group consisting of A1AG1, A1AT, AACT, AMY2B, ANXA1, APOA1, CAH1, CATD, CEAM3, CLUS, CO3, CO9, CRP, CSF1, CTNB1, DPP4, ECH1, FHL1, FIBB, FIBG, FRIL, GELS, HPT, OSTP, PRDX1, SAA1, SBP1, SEPR, SPB6, SPON2, SRC, SYG, TIMP, TIMP1, and TRFE.

24. The method of any one of claims 1-11, wherein the panel comprises at least nine proteins selected from the group consisting of A1AG1, A1AT, AACT, AMY2B, ANXA1, APOA1, CAH1, CATD, CEAM3, CLUS, CO3, CO9, CRP, CSF1, CTNB1, DPP4, ECH1, FHL1, FIBB, FIBG, FRIL, GELS, HPT, OSTP, PRDX1, SAA1, SBP1, SEPR, SPB6, SPON2, SRC, SYG, TIMP, TIMP1, and TRFE.

25. The method of any one of claims 1-11, wherein the panel comprises at least ten proteins selected from the group consisting of A1AG1, A1AT, AACT, AMY2B, ANXA1, APOA1, CAH1, CATD, CEAM3, CLUS, CO3, CO9, CRP, CSF1, CTNB1, DPP4, ECH1, FHL1, FIBB, FIBG, FRIL, GELS, HPT, OSTP, PRDX1, SAA1, SBP1, SEPR, SPB6, SPON2, SRC, SYG, TIMP, TIMP1, and TRFE.

26. The method of any one of claims 1-11, wherein the panel comprises at least eleven proteins selected from the group consisting of A1AG1, A1AT, AACT, AMY2B, ANXA1, APOA1, CAH1, CATD, CEAM3, CLUS, CO3, CO9, CRP, CSF1, CTNB1, DPP4, ECH1, FHL1, FIBB, FIBG, FRIL, GELS, HPT, OSTP, PRDX1, SAA1, SBP1, SEPR, SPB6, SPON2, SRC, SYG, TIMP, TIMP1, and TRFE.

27. The method of any one of claims 1-11, wherein the panel comprises at least twelve proteins selected from the group consisting of A1AG1, A1AT, AACT, AMY2B, ANXA1, APOA1, CAH1, CATD, CEAM3, CLUS, CO3, CO9, CRP, CSF1, CTNB1, DPP4, ECH1, FHL1, FIBB, FIBG, FRIL, GELS, HPT, OSTP, PRDX1, SAA1, SBP1, SEPR, SPB6, SPON2, SRC, SYG, TIMP, TIMP1, and TRFE.

28. The method of any one of claims 1-11, wherein the panel comprises at least thirteen proteins selected from the group consisting of A1AG1, A1AT, AACT, AMY2B, ANXA1, APOA1, CAH1, CATD, CEAM3, CLUS, CO3, CO9, CRP, CSF1, CTNB1, DPP4, ECH1, FHL1, FIBB, FIBG, FRIL, GELS, HPT, OSTP, PRDX1, SAA1, SBP1, SEPR, SPB6, SPON2, SRC, SYG, TIMP, TIMP1, and TRFE.

29. The method of any one of claims 1-11, wherein the panel comprises CO9 and GELS, and wherein the categorizing has an area under the receiver operating characteristic curve greater than 0.76.

30. The method of any one of claims 1-11, wherein the panel comprises A1AG1, A1AT, AACT, ANXA1, APOA1, CO9, CRP, CSF1, FHL1, FIBG, GELS, HPT, and SAM.

31. The method of any one of claims 1-11, wherein the panel comprises A1AG1, A1AT, AACT, ANXA1, APOA1, CO9, CRP, CSF1, FHL1, FIBG, GELS, HPT, and SAM, and wherein the categorizing has an area under the receiver operating characteristic curve greater than 0.83.

32. The method of any one of claims 1-11, wherein the panel comprises APOA1, CO3, and CO9.

33. The method of any one of claims 1-11, wherein the panel comprises APOA1, CO3, and CO9, and wherein the categorizing has an area under the receiver operating characteristic curve greater than 0.81.

34. The method of any one of claims 1-11, wherein the panel comprises AACT, CO3, CO9, CRP, and GELS.

35. The method of any one of claims 1-11, wherein the panel comprises AACT, CO3, CO9, CRP, and GELS, and wherein the categorizing has an area under the receiver operating characteristic curve greater than 0.81.

36. The method of any one of claims 1-11, wherein the panel comprises SPB6, GELS, A1AT, FIBG, and CO3.

37. The method of any one of claims 1-11, wherein the panel comprises SPB6, GELS, A1AT, FIBG, and CO3, and wherein the categorizing has an area under the receiver operating characteristic curve greater than 0.79.

38. The method of any one of claims 1-11, wherein the panel comprises A1AG1, A1AT, AMY2B, CLUS, CO9, ECH1, FRIL, GELS, OSTP, SBP1, SEPR, SPON2, and TIMP1.

39. The method of any one of claims 1-11, wherein the panel comprises A1AG1, A1AT, AMY2B, CLUS, CO9, ECH1, FRIL, GELS, OSTP, SBP1, SEPR, SPON2, and TIMP1, and wherein the categorizing has an area under the receiver operating characteristic curve greater than 0.91.

40. The method of any one of claims 1-11, wherein the panel comprises CO9 and GELS.

41. The method of any one of claims 1-11, wherein the panel comprises CO9 and GELS, and wherein the categorizing has an area under the receiver operating characteristic curve greater than 0.87.

42. The method of any one of claims 1-11, wherein the panel comprises GELS, PRDX1, CO9, and CATD.

43. The method of any one of claims 1-11, wherein the panel comprises GELS, PRDX1, CO9, and CATD, and wherein the categorizing has an area under the receiver operating characteristic curve greater than 0.84.

44. The method of any one of claims 1-11, wherein the panel comprises A1AT, APOA1, and FIBG.

45. The method of any one of claims 1-11, wherein the panel comprises A1AT, APOA1, and FIBG, and wherein the categorizing has an area under the receiver operating characteristic curve greater than 0.81.

46. The method of any one of claims 1-11, wherein the panel comprises A1AT and TRFE.

47. The method of any one of claims 1-11, wherein the panel comprises A1AT and TRFE, and wherein the categorizing has an area under the receiver operating characteristic curve greater than 0.89.

48. The method of any one of claims 1-11, wherein the panel comprises A1AT, APOA1, FIBB, and CEAM3.

49. The method of any one of claims 1-11, wherein the panel comprises A1AT, APOA1, FIBB, and CEAM3, and wherein the categorizing has an area under the receiver operating characteristic curve greater than 0.80.

50. The method of any one of claims 1-11, wherein the panel comprises CAH1, CRP, FIBG, and CTNB1.

51. The method of any one of claims 1-11, wherein the panel comprises CAH1, CRP, FIBG, and CTNB1, and wherein the categorizing has an area under the receiver operating characteristic curve greater than 0.78.

52. The method of any one of claims 1-11, wherein the panel comprises CRP, SEPR, SBP1, SRC, and DPP4.

53. The method of any one of claims 1-11, wherein the panel comprises CRP, SEPR, SBP1, SRC, and DPP4, and wherein the categorizing has an area under the receiver operating characteristic curve greater than 0.78.

54. The method of any one of claims 1-11, wherein the panel comprises CRP and TMP.

55. The method of any one of claims 1-11, wherein the panel comprises CRP and TMP, and wherein the categorizing has an area under the receiver operating characteristic curve greater than 0.76.

56. The method of any one of claims 1-11, wherein the panel comprises SYG, AACT, and CO9.

57. The method of any one of claims 1-11, wherein the panel comprises SYG, AACT, and CO9, and wherein the categorizing has an area under the receiver operating characteristic curve greater than 0.86.

58. The method of any one of claims 1-11, wherein the panel comprises CATD, CEA, CO9, and SEPR.

59. The method of any one of claims 1-58, further comprising providing a recommendation correlating to said categorization.

60. The method of any one of claims 1-59, wherein the categorizing is as a cancer-free individual.

61. The method of claim 59, wherein the recommendation comprises continued monitoring.

62. The method of any one of claims 1-59, wherein the categorization is as an individual having CRC.

63. The method of claim 62, wherein the recommendation comprises undergoing a colonoscopy.

64. The method of claim 62, wherein the recommendation comprises undergoing a sigmoidoscopy.

65. The method of claim 62, wherein the recommendation comprises undergoing an independent cancer assay.

66. The method of claim 62, wherein the recommendation comprises undergoing a stool cancer assay.

67. The method of claim 62, wherein the recommendation comprises undergoing chemotherapy.

68. The method of claim 62, wherein the recommendation comprises administration of a biologic anticancer agent.

69. The method of claim 62, wherein the recommendation comprises a surgical intervention.

70. A method for monitoring an individual's response to a colorectal cancer treatment regimen administered to said individual, comprising
comparing accumulation levels of a panel of proteins in a blood sample obtained from said individual at a first time period to accumulation levels of a panel of proteins in a blood sample obtained from said individual at a second time period.

71. The method of claim 70, wherein said monitoring comprises subjecting said blood sample to an ELISA assay.

72. The method of claim 70, wherein said monitoring comprises subjecting said blood sample to a mass spectrometric assay.

73. The method of claim 70, wherein the panel comprises A1AG1, A1AT, CATD, CEA, CO9, OSTP, and SEPR.

74. The method of claim 70, wherein the panel comprises A1AG1, A1AT, APOA1, CATD, CEA, CLUS, CO3, CO9, FGB, FIBG, GELS, PRDX1, SBP1, and SEPR.

75. The method of claim 70, wherein the panel comprises A1AG1, A1AT, CATD, CEA, CO9, and SEPR.

76. The method of claim 70, wherein the panel comprises A1AG1, A1AT, AACT, CATD, CEA, CO9, CRP, GELS, SAA1, and SEPR.

77. The method of claim 70, wherein the panel comprises CATD, CEA, CO3, CO9, GELS, and SEPR.

78. The method of claim 70, wherein the panel comprises CATD, CEA, CO9, and SEPR.

79. The method of claim 70, wherein the panel comprises at least two proteins selected from the group consisting of A1AG1, A1AT, AACT, AMY2B, ANXA1, APOA1, CAH1, CATD, CEAM3, CLUS, CO3, CO9, CRP, CSF1, CTNB1, DPP4, ECH1, FHL1, FIBB, FIBG, FRIL, GELS, HPT, OSTP, PRDX1, SAA1, SBP1, SEPR, SPB6, SPON2, SRC, SYG, TIMP, TIMP1, and TRFE.

80. The method of claim 70, wherein the panel comprises at least three proteins selected from the group consisting of A1AG1, A1AT, AACT, AMY2B, ANXA1, APOA1, CAH1, CATD, CEAM3, CLUS, CO3, CO9, CRP, CSF1, CTNB1, DPP4, ECH1, FHL1, FIBB, FIBG, FRIL, GELS, HPT, OSTP, PRDX1, SAA1, SBP1, SEPR, SPB6, SPON2, SRC, SYG, TIMP, TIMP1, and TRFE.

81. The method of claim 70, wherein the panel comprises at least four proteins selected from the group consisting of A1AG1, A1AT, AACT, AMY2B, ANXA1, APOA1, CAH1, CATD, CEAM3, CLUS, CO3, CO9, CRP, CSF1, CTNB1, DPP4, ECH1, FHL1, FIBB, FIBG, FRIL, GELS, HPT, OSTP, PRDX1, SAA1, SBP1, SEPR, SPB6, SPON2, SRC, SYG, TIMP, TIMP1, and TRFE.

82. The method of claim 70, wherein the panel comprises at least five proteins selected from the group consisting of A1AG1, A1AT, AACT, AMY2B, ANXA1, APOA1, CAH1, CATD, CEAM3, CLUS, CO3, CO9, CRP, CSF1, CTNB1, DPP4, ECH1, FHL1, FIBB, FIBG, FRIL, GELS, HPT, OSTP, PRDX1, SAA1, SBP1, SEPR, SPB6, SPON2, SRC, SYG, TIMP, TIMP1, and TRFE.

83. The method of claim 70, wherein the panel comprises at least six proteins selected from the group consisting of A1AG1, A1AT, AACT, AMY2B, ANXA1, APOA1, CAH1, CATD, CEAM3, CLUS, CO3, CO9, CRP, CSF1, CTNB1, DPP4, ECH1, FHL1, FIBB, FIBG, FRIL, GELS, HPT, OSTP, PRDX1, SAA1, SBP1, SEPR, SPB6, SPON2, SRC, SYG, TIMP, TIMP1, and TRFE.

84. The method of claim 70, wherein the panel comprises at least seven proteins selected from the group consisting of A1AG1, A1AT, AACT, AMY2B, ANXA1, APOA1, CAH1, CATD, CEAM3, CLUS, CO3, CO9, CRP, CSF1, CTNB1, DPP4, ECH1, FHL1, FIBB, FIBG, FRIL, GELS, HPT, OSTP, PRDX1, SAA1, SBP1, SEPR, SPB6, SPON2, SRC, SYG, TIMP, TIMP1, and TRFE.

85. The method of claim 70, wherein the panel comprises at least eight proteins selected from the group consisting of A1AG1, A1AT, AACT, AMY2B, ANXA1, APOA1, CAH1, CATD, CEAM3, CLUS, CO3, CO9, CRP, CSF1, CTNB1, DPP4, ECH1, FHL1, FIBB, FIBG, FRIL, GELS, HPT, OSTP, PRDX1, SAA1, SBP1, SEPR, SPB6, SPON2, SRC, SYG, TIMP, TIMP1, and TRFE.

86. The method of claim 70, wherein the panel comprises at least nine proteins selected from the group consisting of A1AG1, A1AT, AACT, AMY2B, ANXA1, APOA1, CAH1, CATD, CEAM3, CLUS, CO3, CO9, CRP, CSF1, CTNB1, DPP4, ECH1, FHL1, FIBB, FIBG, FRIL, GELS, HPT, OSTP, PRDX1, SAA1, SBP1, SEPR, SPB6, SPON2, SRC, SYG, TIMP, TIMP1, and TRFE.

87. The method of claim 70, wherein the panel comprises at least ten proteins selected from the group consisting of A1AG1, A1AT, AACT, AMY2B, ANXA1, APOA1, CAH1, CATD, CEAM3, CLUS, CO3, CO9, CRP, CSF1, CTNB1, DPP4, ECH1, FHL1, FIBB, FIBG, FRIL, GELS, HPT, OSTP, PRDX1, SAA1, SBP1, SEPR, SPB6, SPON2, SRC, SYG, TIMP, TIMP1, and TRFE.

88. The method of claim 70, wherein the panel comprises at least eleven proteins selected from the group consisting of A1AG1, A1AT, AACT, AMY2B, ANXA1, APOA1, CAH1, CATD, CEAM3, CLUS, CO3, CO9, CRP, CSF1, CTNB1, DPP4, ECH1, FHL1, FIBB, FIBG, FRIL, GELS, HPT, OSTP, PRDX1, SAA1, SBP1, SEPR, SPB6, SPON2, SRC, SYG, TIMP, TIMP1, and TRFE.

89. The method of claim 70, wherein the panel comprises at least twelve proteins selected from the group consisting of A1AG1, A1AT, AACT, AMY2B, ANXA1, APOA1, CAH1, CATD, CEAM3, CLUS, CO3, CO9, CRP, CSF1, CTNB1, DPP4, ECH1, FHL1, FIBB, FIBG, FRIL, GELS, HPT, OSTP, PRDX1, SAA1, SBP1, SEPR, SPB6, SPON2, SRC, SYG, TIMP, TIMP1, and TRFE.

90. The method of claim 70, wherein the panel comprises at least thirteen proteins selected from the group consisting of A1AG1, A1AT, AACT, AMY2B, ANXA1, APOA1, CAH1, CATD, CEAM3, CLUS, CO3, CO9, CRP, CSF1, CTNB1, DPP4, ECH1, FHL1, FIBB, FIBG, FRIL, GELS, HPT, OSTP, PRDX1, SAA1, SBP1, SEPR, SPB6, SPON2, SRC, SYG, TIMP, TIMP1, and TRFE.

91. The method of claim 70, wherein the panel comprises CO9 and GELS.

92. The method of claim 70, wherein the panel comprises CO9 and GELS, and wherein the categorizing has an area under the receiver operating characteristic curve greater than 0.76.

93. The method of claim 70, wherein the panel comprises A1AG1, A1AT, AACT, ANXA1, APOA1, CO9, CRP, CSF1, FHL1, FIBG, GELS, HPT, and SAM.

94. The method of claim 70, wherein the panel comprises A1AG1, A1AT, AACT, ANXA1, APOA1, CO9, CRP, CSF1, FHL1, FIBG, GELS, HPT, and SAM, and wherein the categorizing has an area under the receiver operating characteristic curve greater than 0.83.

95. The method of claim 70, wherein the panel comprises APOA1, CO3, and CO9.

96. The method of claim 70, wherein the panel comprises APOA1, CO3, and CO9, and wherein the categorizing has an area under the receiver operating characteristic curve greater than 0.81.

97. The method of claim 70, wherein the panel comprises AACT, CO3, CO9, CRP, and GELS.

98. The method of claim 70, wherein the panel comprises AACT, CO3, CO9, CRP, and GELS, and wherein the categorizing has an area under the receiver operating characteristic curve greater than 0.81.

99. The method of claim 70, wherein the panel comprises SPB6, GELS, A1AT, FIBG, and CO3.

100. The method of claim 70, wherein the panel comprises SPB6, GELS, A1AT, FIBG, and CO3, and wherein the categorizing has an area under the receiver operating characteristic curve greater than 0.79.

101. The method of claim 70, wherein the panel comprises A1AG1, A1AT, AMY2B, CLUS, CO9, ECH1, FRIL, GELS, OSTP, SBP1, SEPR, SPON2, and TIMP1.

102. The method of claim 70, wherein the panel comprises A1AG1, A1AT, AMY2B, CLUS, CO9, ECH1, FRIL, GELS, OSTP, SBP1, SEPR, SPON2, and TIMP1, and wherein the categorizing has an area under the receiver operating characteristic curve greater than 0.91.

103. The method of claim 70, wherein the panel comprises CO9 and GELS, and wherein the categorizing has an area under the receiver operating characteristic curve greater than 0.87.

104. The method of claim 70, wherein the panel comprises GELS, PRDX1, CO9, and CATD.

105. The method of claim 70, wherein the panel comprises GELS, PRDX1, CO9, and CATD, and wherein the categorizing has an area under the receiver operating characteristic curve greater than 0.84.

106. The method of claim 70, wherein the panel comprises A1AT, APOA1, and FIBG.

107. The method of claim 70, wherein the panel comprises A1AT, APOA1, and FIBG, and wherein the categorizing has an area under the receiver operating characteristic curve greater than 0.81.

108. The method of claim 70, wherein the panel comprises A1AT and TRFE.

109. The method of claim 70, wherein the panel comprises A1AT and TRFE, and wherein the categorizing has an area under the receiver operating characteristic curve greater than 0.89.

110. The method of claim 70, wherein the panel comprises A1AT, APOA1, FIBB, and CEAM3.

111. The method of claim 70, wherein the panel comprises A1AT, APOA1, FIBB, and CEAM3, and wherein the categorizing has an area under the receiver operating characteristic curve greater than 0.80.

112. The method of claim 70, wherein the panel comprises CAH1, CRP, FIBG, and CTNB1.

113. The method of claim 70, wherein the panel comprises CAH1, CRP, FIBG, and CTNB1, and wherein the categorizing has an area under the receiver operating characteristic curve greater than 0.78.

114. The method of claim 70, wherein the panel comprises CRP, SEPR, SBP1, SRC, and DPP4.

115. The method of claim 70, wherein the panel comprises CRP, SEPR, SBP1, SRC, and DPP4, and wherein the categorizing has an area under the receiver operating characteristic curve greater than 0.78.

116. The method of claim 70, wherein the panel comprises CRP and TMP.

117. The method of claim 70, wherein the panel comprises CRP and TMP, and wherein the categorizing has an area under the receiver operating characteristic curve greater than 0.76.

118. The method of claim 70, wherein the panel comprises SYG, AACT, and CO9.

119. The method of claim 70, wherein the panel comprises SYG, AACT, and CO9, and wherein the categorizing has an area under the receiver operating characteristic curve greater than 0.86.

120. The method of claim 70, wherein the sample obtained at a first time period comprises serum.

121. The method of claim 70, wherein the sample obtained at a first time period comprises blood proteins.

122. The method of claim 70, wherein the sample obtained at a first time period comprises proteins obtained from a blood sample.

123. The method of any one of claims 70-122, further comprising comparing said accumulation levels of a panel of proteins in a blood sample obtained from said individual at said first time period to accumulation levels of the panel of proteins in a blood sample obtained from said individual at a third time period.

124. The method of any one of claims 70-123, wherein said first sample is obtained prior to initiation of said treatment regimen.

125. A kit comprising a panel of antibodies for use in an ELISA assay to assess colorectal cancer status.

126. The kit of claim 125, wherein the panel of antibodies comprises antibodies to A1AG1, A1AT, CATD, CEA, CO9, OSTP, and SEPR.

127. The kit of claim 125, wherein the panel of antibodies comprises antibodies to A1AG1, A1AT, APOA1, CATD, CEA, CLUS, CO3, CO9, FGB, FIBG, GELS, PRDX1, SBP1, and SEPR.

128. The kit of claim 125, wherein the panel of antibodies comprises antibodies to A1AG1, A1AT, CATD, CEA, CO9, and SEPR.

129. The kit of claim 125, wherein the panel of antibodies comprises antibodies to A1AG1, A1AT, AACT, CATD, CEA, CO9, CRP, GELS, SAA1, and SEPR.

130. The kit of claim 125, wherein the panel of antibodies comprises antibodies to CATD, CEA, CO3, CO9, GELS, and SEPR.

131. The kit of claim 125, wherein the panel of antibodies comprises antibodies to CATD, CEA, CO9, and SEPR.

132. The kit of claim 125, wherein the panel of antibodies comprises antibodies to at least two biomarkers selected from the group consisting of A1AG1, A1AT, AACT, AMY2B, ANXA1, APOA1, CAH1, CATD, CEAM3, CLUS, CO3, CO9, CRP, CSF1, CTNB1, DPP4, ECH1, FHL1, FIBB, FIBG, FRIL, GELS, HPT, OSTP, PRDX1, SAA1, SBP1, SEPR, SPB6, SPON2, SRC, SYG, TIMP, TIMP1, and TRFE.

133. The kit of claim 125, wherein the panel of antibodies comprises antibodies to at least three biomarkers selected from the group consisting of A1AG1, A1AT, AACT, AMY2B, ANXA1, APOA1, CAH1, CATD, CEAM3, CLUS, CO3, CO9, CRP, CSF1, CTNB1, DPP4, ECH1, FHL1, FIBB, FIBG, FRIL, GELS, HPT, OSTP, PRDX1, SAA1, SBP1, SEPR, SPB6, SPON2, SRC, SYG, TIMP, TIMP1, and TRFE.

134. The kit of claim 125, wherein the panel of antibodies comprises antibodies to at least four biomarkers selected from the group consisting of A1AG1, A1AT, AACT, AMY2B, ANXA1, APOA1, CAH1, CATD, CEAM3, CLUS, CO3, CO9, CRP, CSF1, CTNB1, DPP4, ECH1, FHL1, FIBB, FIBG, FRIL, GELS, HPT, OSTP, PRDX1, SAA1, SBP1, SEPR, SPB6, SPON2, SRC, SYG, TIMP, TIMP1, and TRFE.

135. The kit of claim 125, wherein the panel of antibodies comprises antibodies to at least five biomarkers selected from the group consisting of A1AG1, A1AT, AACT, AMY2B, ANXA1, APOA1, CAH1, CATD, CEAM3, CLUS, CO3, CO9, CRP, CSF1, CTNB1, DPP4, ECH1, FHL1, FIBB, FIBG, FRIL, GELS, HPT, OSTP, PRDX1, SAA1, SBP1, SEPR, SPB6, SPON2, SRC, SYG, TIMP, TIMP1, and TRFE.

136. The kit of claim 125, wherein the panel of antibodies comprises antibodies to at least six biomarkers selected from the group consisting of A1AG1, A1AT, AACT, AMY2B, ANXA1, APOA1, CAH1, CATD, CEAM3, CLUS, CO3, CO9, CRP, CSF1, CTNB1, DPP4, ECH1, FHL1, FIBB, FIBG, FRIL, GELS, HPT, OSTP, PRDX1, SAA1, SBP1, SEPR, SPB6, SPON2, SRC, SYG, TIMP, TIMP1, and TRFE.

137. The kit of claim 125, wherein the panel of antibodies comprises antibodies to at least seven biomarkers selected from the group consisting of A1AG1, A1AT, AACT, AMY2B, ANXA1, APOA1, CAH1, CATD, CEAM3, CLUS, CO3, CO9, CRP, CSF1, CTNB1, DPP4, ECH1, FHL1, FIBB, FIBG, FRIL, GELS, HPT, OSTP, PRDX1, SAA1, SBP1, SEPR, SPB6, SPON2, SRC, SYG, TIMP, TIMP1, and TRFE.

138. The kit of claim 125, wherein the panel of antibodies comprises antibodies to at least eight biomarkers selected from the group consisting of A1AG1, A1AT, AACT, AMY2B, ANXA1, APOA1, CAH1, CATD, CEAM3, CLUS, CO3, CO9, CRP, CSF1, CTNB1, DPP4, ECH1, FHL1, FIBB, FIBG, FRIL, GELS, HPT, OSTP, PRDX1, SAA1, SBP1, SEPR, SPB6, SPON2, SRC, SYG, TIMP, TIMP1, and TRFE.

139. The kit of claim 125, wherein the panel of antibodies comprises antibodies to at least nine biomarkers selected from the group consisting of A1AG1, A1AT, AACT, AMY2B, ANXA1, APOA1, CAH1, CATD, CEAM3, CLUS, CO3, CO9, CRP, CSF1, CTNB1, DPP4, ECH1, FHL1, FIBB, FIBG, FRIL, GELS, HPT, OSTP, PRDX1, SAA1, SBP1, SEPR, SPB6, SPON2, SRC, SYG, TIMP, TIMP1, and TRFE.

140. The kit of claim 125, wherein the panel of antibodies comprises antibodies to at least ten biomarkers selected from the group consisting of A1AG1, A1AT, AACT, AMY2B, ANXA1, APOA1, CAH1, CATD, CEAM3, CLUS, CO3, CO9, CRP, CSF1, CTNB1, DPP4, ECH1, FHL1, FIBB, FIBG, FRIL, GELS, HPT, OSTP, PRDX1, SAA1, SBP1, SEPR, SPB6, SPON2, SRC, SYG, TIMP, TIMP1, and TRFE.

141. The kit of claim 125, wherein the panel of antibodies comprises antibodies to at least eleven biomarkers selected from the group consisting of A1AG1, A1AT, AACT, AMY2B, ANXA1, APOA1, CAH1, CATD, CEAM3, CLUS, CO3, CO9, CRP, CSF1, CTNB1, DPP4, ECH1, FHL1, FIBB, FIBG, FRIL, GELS, HPT, OSTP, PRDX1, SAA1, SBP1, SEPR, SPB6, SPON2, SRC, SYG, TIMP, TIMP1, and TRFE.

142. The kit of claim 125, wherein the panel of antibodies comprises antibodies to at least twelve biomarkers selected from the group consisting of A1AG1, A1AT, AACT, AMY2B, ANXA1, APOA1, CAH1, CATD, CEAM3, CLUS, CO3, CO9, CRP, CSF1, CTNB1, DPP4, ECH1, FHL1, FIBB, FIBG, FRIL, GELS, HPT, OSTP, PRDX1, SAA1, SBP1, SEPR, SPB6, SPON2, SRC, SYG, TIMP, TIMP1, and TRFE.

143. The kit of claim 125, wherein the panel of antibodies comprises antibodies to at least thirteen biomarkers selected from the group consisting of A1AG1, A1AT, AACT, AMY2B, ANXA1, APOA1, CAH1, CATD, CEAM3, CLUS, CO3, CO9, CRP, CSF1, CTNB1, DPP4, ECH1, FHL1, FIBB, FIBG, FRIL, GELS, HPT, OSTP, PRDX1, SAA1, SBP1, SEPR, SPB6, SPON2, SRC, SYG, TIMP, TIMP1, and TRFE.

144. The kit of claim 125, wherein the panel of antibodies comprises antibodies to A1AG1, A1AT, AACT, ANXA1, APOA1, CO9, CRP, CSF1, FHL1, FIBG, GELS, HPT, and SAM.

145. The kit of claim 125, wherein the panel of antibodies comprises antibodies to APOA1, CO3, and CO9.

146. The kit of claim 125, wherein the panel of antibodies comprises antibodies to AACT, CO3, CO9, CRP, and GELS.

147. The kit of claim 125, wherein the panel of antibodies comprises antibodies to SPB6, GELS, A1AT, FIBG, and CO3.

148. The kit of claim 125, wherein the panel of antibodies comprises antibodies to A1AG1, A1AT, AMY2B, CLUS, CO9, ECH1, FRIL, GELS, OSTP, SBP1, SEPR, SPON2, and TIMP1.

149. The kit of claim 125, wherein the panel of antibodies comprises antibodies to CO9 and GELS.

150. The kit of claim 125, wherein the panel of antibodies comprises antibodies to GELS, PRDX1, CO9, and CATD.

151. The kit of claim 125, wherein the panel of antibodies comprises antibodies to A1AT, APOA1, and FIBG.

152. The kit of claim 125, wherein the panel of antibodies comprises antibodies to A1AT and TRFE.

153. The kit of claim 125, wherein the panel of antibodies comprises antibodies to A1AT, APOA1, FIBB, and CEAM3.

154. The kit of claim 125, wherein the panel of antibodies comprises antibodies to CAH1, CRP, FIBG, and CTNB1.

155. The kit of claim 125, wherein the panel of antibodies comprises antibodies to CRP, SEPR, SBP1, SRC, and DPP4.

156. The kit of claim 125, wherein the panel of antibodies comprises antibodies to CRP and TMP.

157. The kit of claim 125, wherein the panel of antibodies comprises antibodies to SYG, AACT, and CO9.

158. The kit of claim 125, wherein the ELISA assay to assess colorectal cancer status has a sensitivity of at least 80%.

159. The kit of claim 125, wherein the ELISA assay to assess colorectal cancer status has a sensitivity of at least 85%.

160. The kit of claim 125, wherein the ELISA assay to assess colorectal cancer status has a sensitivity of at least 90%.

161. The kit of claim 125, wherein the ELISA assay to assess colorectal cancer status has a specificity of at least 80%.

162. The kit of claim 125, wherein the ELISA assay to assess colorectal cancer status has a specificity of at least 85%.

163. The kit of claim 125, wherein the ELISA assay to assess colorectal cancer status has a specificity of at least 90%.

164. The kit of any one of claims 125-163, wherein the kit comprises reagents for extracting blood from an individual, and reagents for extracting proteins from blood.

165. A method of treating at least one of a colorectal cancer and advanced colorectal adenoma in a subject, comprising:
(a) measuring a biomarker panel in a biological sample obtained from the subject, wherein the biomarker panel comprises at least two biomarkers selected from the group consisting of A1AG1, A1AT, AACT, AMY2B, ANXA1, APOA1, CAH1, CATD, CEAM3, CLUS, CTNB1, CO3, CO9, CRP, CSF1, DPP4, ECH1, FHL1, FIBB, FIBG, FRIL, GELS, HPT, OSTP, PRDX1, SAA1, SBP1, SEPR, SPB6, SPON2, SYG, TIMP1, and TRFE;
(b) detecting a presence or absence of colorectal cancer and/or advanced colorectal adenoma in the subject based upon the measuring; and
(c) treating the colorectal cancer in the subject based upon the detecting.

166. A method of diagnosing at least one of a colorectal cancer and advanced colorectal adenoma in a subject, comprising:
(a) measuring a biomarker panel in a biological sample obtained from the subject, wherein the biomarker panel comprises at least two biomarkers selected from the group consisting of A1AG1, A1AT, AACT, AMY2B, ANXA1, APOA1, CAH1, CATD, CEAM3, CLUS, CTNB1, CO3, CO9, CRP, CSF1, DPP4, ECH1, FHL1, FIBB, FIBG, FRIL, GELS, HPT, OSTP, PRDX1, SAA1, SBP1, SEPR, SPB6, SPON2, SYG, TIMP1, and TRFE;
(b) detecting a presence or absence of at least one of colorectal cancer and advanced colorectal adenoma in the subject based upon the measuring; and
(c) recommending to the subject at least one of a colonoscopy, sigmoidoscopy, and tissue biopsy in the subject based upon the detecting.

167. A method, comprising:
(a) obtaining data comprising a measurement of a biomarker panel in a biological sample obtained from a subject, wherein the biomarker panel comprises at least two biomarkers selected from the group consisting of A1AG1, A1AT, AACT, AMY2B, ANXA1, APOA1, CAH1, CATD, CEAM3, CLUS, CTNB1, CO3, CO9, CRP, CSF1, DPP4, ECH1, FHL1, FIBB, FIBG, FRIL, GELS, HPT, OSTP, PRDX1, SAA1, SBP1, SEPR, SPB6, SPON2, SYG, TIMP1, and TRFE;
(b) generating a subject-specific profile of the biomarker panel based upon the measurement data;
(c) comparing the subject-specific profile of the biomarker panel to a reference profile of the biomarker panel; and
(d) determining a likelihood of at least one of advanced colorectal adenoma and colorectal cancer based upon (c).

168. The method of claim 167, comprising detecting a presence or absence of advanced colorectal adenoma in the subject.

169. The method of claim 168, wherein the advanced colorectal adenoma comprises a dimension that is greater than or equal to 1 centimeter.

170. The method of claim 168, wherein the advanced colorectal adenoma is of villous character.

171. The method of any of claims 168-170, comprising detecting a presence or absence of the advanced colorectal adenoma with a sensitivity that is greater than 70%.

172. The method of any of claims 168-171, comprising detecting a presence or absence of the advanced colorectal adenoma with a sensitivity that is greater than 75%, 80%, 85%, 90%, or 95%.

173. The method of any of claims 168-172, further comprising removing the advanced colorectal adenoma from the subject, thereby preventing development of colorectal cancer in the subject.

174. The method of any of claims 168-173, wherein the biomarker panel comprises CATD and FUCO.

175. The method of claim 174, comprising detecting a presence of an advanced colorectal adenoma in the subject if a level of at least one of CATD and FUCO in the biological sample obtained from the subject differs from a negative control reference level of the least one of CATD and FUCO by at least 10%.

176. The method of claim 174, comprising detecting a presence of an advanced colorectal adenoma in the subject if a level of at least one of CATD and FUCO in the biological sample obtained from the subject differs from a positive control reference level of the least one of CATD and FUCO by less than 10%.

177. The method of claim 174, wherein the biomarker panel comprises three biomarkers.

178. The method of claim 177, wherein the three biomarkers are CATD, CATS, and FUCO.

179. The method of claim 178, comprising detecting a presence of an advanced colorectal adenoma in the subject if a level of at least one of CATD, CATS, and FUCO in the biological sample obtained from the subject differs from a negative control reference level of the least one of CATD, CATS, and FUCO by at least 10%.

180. The method of claim 178, comprising detecting a presence of an advanced colorectal adenoma in the subject if a level of at least one of CATD, CATS, and FUCO in the biological sample obtained from the subject differs from a positive control reference level of the least one of CATD, CATS, and FUCO by less than 10%.

181. The method of claim 177, wherein the three biomarkers are CATD, FUCO, and FIBB.

182. The method of claim 181, comprising detecting a presence of an advanced colorectal adenoma in the subject if a level of at least one of CATD, FUCO, and FIBB in the biological sample obtained from the subject differs from a negative control reference level of the least one of CATD, FUCO, and FIBB by at least 10%.

183. The method of claim 181, comprising detecting a presence of an advanced colorectal adenoma in the subject if a level of at least one of CATD, FUCO, and FIBB in the biological sample obtained from the subject differs from a positive control reference level of the least one of CATD, FUCO, and FIBB by less than 10%.

184. The method of claim 177, wherein the three biomarkers are CATD, FUCO, and SAHH.

185. The method of claim 184, comprising detecting a presence of an advanced colorectal adenoma in the subject if a level of at least one of CATD, FUCO, and SAHH in the biological sample obtained from the subject differs from a negative control reference level of the least one of CATD, FUCO, and SAHH by at least 10%.

186. The method of claim 184, comprising detecting a presence of an advanced colorectal adenoma in the subject if a level of at least one of CATD, FUCO, and SAHH in the biological sample obtained from the subject differs from a positive control reference level of the least one of CATD, FUCO, and SAHH by less than 10%.

187. The method of any one of claims 177-186, wherein the biomarker panel comprises no more than three biomarkers.

188. The method of claim 174, wherein the biomarker panel comprises four biomarkers.

189. The method of claim 188, wherein the four biomarkers are CATD, FIBB, FUCO, and SAHH.

190. The method of claim 189, comprising detecting a presence of an advanced colorectal adenoma in the subject if a level of at least one of CATD, FIBB, FUCO, and SAHH in the biological sample obtained from the subject differs from a negative control reference level of the least one of CATD, FIBB, FUCO, and SAHH by at least 10%.

191. The method of claim 189, comprising detecting a presence of an advanced colorectal adenoma in the subject if a level of at least one of CATD, FIBB, FUCO, and SAHH in the biological sample obtained from the subject differs from a positive control reference level of the least one of CATD, FIBB, FUCO, and SAHH by less than 10%.

192. The method of claim 167, comprising detecting a presence or absence of colorectal cancer in the subject.

193. The method of claim 192, wherein the biomarker panel comprises CO9 and GELS.

194. The method of claim 193, comprising detecting a presence of colorectal cancer in the subject if a level of at least one of CO9 and GELS in the biological sample obtained from the subject differs from a negative control reference level of the least one of CO9 and GELS by at least 10%.

195. The method of claim 193, comprising detecting a presence of colorectal cancer in the subject if a level of at least one of CO9 and GELS in the biological sample obtained from the subject differs from a positive control reference level of the least one of CO9 and GELS by less than 10%.

196. The method of claim 192, wherein the biomarker panel comprises at least three biomarkers.

197. The method of claim 196, wherein the at least three biomarkers are AACT, CO9, and SYG.

198. The method of claim 197, comprising detecting a presence of colorectal cancer in the subject if a level of at least one of AACT, CO9, and SYG in the biological sample obtained from the subject differs from a negative control reference level of the least one of AACT, CO9, and SYG by at least 10%.

199. The method of claim 197, comprising detecting a presence of colorectal cancer in the subject if a level of at least one of AACT, CO9, and SYG in the biological sample obtained from the subject differs from a positive control reference level of the least one of AACT, CO9, and SYG by less than 10%.

200. The method of claim 192, wherein the biomarker panel comprises CRP and TIMP1.

201. The method of claim 200, comprising detecting a presence of colorectal cancer in the subject if a level of at least one of CRP and TIMP1 in the biological sample obtained from the subject differs from a negative control reference level of the least one of CRP and TIMP1 by at least 10%.

202. The method of claim 200, comprising detecting a presence of colorectal cancer in the subject if a level of at least one of CRP and TIMP1 in the biological sample obtained from the subject differs from a positive control reference level of the least one of CRP and TIMP1 by less than 10%.

203. The method of claim 192, wherein the biomarker panel comprises at least four biomarkers.

204. The method of claim 203, wherein the at least four biomarkers comprise CO9, GELS, PRDX1, and CATD.

205. The method of claim 204, comprising detecting a presence of colorectal cancer in the subject if a level of at least one of CO9, GELS, PRDX1, and CATD in the biological sample obtained from the subject differs from a negative control reference level of the least one of CO9, GELS, PRDX1, and CATD by at least 10%.

206. The method of claim 204, comprising detecting a presence of colorectal cancer in the subject if a level of at least one of CO9, GELS, PRDX1, and CATD in the biological sample obtained from the subject differs from a positive control reference level of the least one of CO9, GELS, PRDX1, and CATD by less than 10%.

207. The method of claim 203, wherein the at least four biomarkers comprise A1AT, APOA1, FIBB, and CEAM3.

208. The method of claim 207, comprising detecting a presence of colorectal cancer in the subject if a level of at least one of A1AT, APOA1, FIBB, and CEAM3 in the biological sample obtained from the subject differs from a negative control reference level of the least one of A1AT, APOA1, FIBB, and CEAM3 by at least 10%.

209. The method of claim 207, comprising detecting a presence of colorectal cancer in the subject if a level of at least one of A1AT, APOA1, FIBB, and CEAM3 in the biological sample obtained from the subject differs from a positive control reference level of the least one of A1AT, APOA1, FIBB, and CEAM3 by less than 10%.

210. The method of claim 203, wherein the at least four biomarkers comprise CAH1, CRP, FIBG, and CTNB1.

211. The method of claim 210, comprising detecting a presence of colorectal cancer in the subject if a level of at least one of CAH1, CRP, FIBG, and CTNB1 in the biological sample obtained from the subject differs from a negative control reference level of the least one of CAH1, CRP, FIBG, and CTNB1 by at least 10%.

212. The method of claim 210, comprising detecting a presence of colorectal cancer in the subject if a level of at least one of CAH1, CRP, FIBG, and CTNB1 in the biological sample obtained from the subject differs from a positive control reference level of the least one of CAH1, CRP, FIBG, and CTNB1 by less than 10%.

213. The method of claim 203, wherein the at least four biomarkers comprise A1AG1, A1AT, CO9, and GELS.

214. The method of claim 213, comprising detecting a presence of colorectal cancer in the subject if a level of at least one of A1AG1, A1AT, CO9, and GELS in the biological sample obtained from the subject differs from a negative control reference level of the least one of A1AG1, A1AT, CO9, and GELS by at least 10%.

215. The method of claim 213, comprising detecting a presence of colorectal cancer in the subject if a level of at least one of A1AG1, A1AT, CO9, and GELS in the biological sample obtained from the subject differs from a positive control reference level of the least one of A1AG1, A1AT, CO9, and GELS by less than 10%.

216. The method of claim 192, wherein the biomarker panel comprises 13 biomarkers.

217. The method of claim 216, wherein the 13 biomarkers are A1AG1, A1AT, AACT, ANXA1, APOA1, CO9, CRP, CSF1, FHL1, FIBG, GELS, HPT, and SAM.

218. The method of claim 217, comprising detecting a presence of colorectal cancer in the subject if a level of at least one of A1AG1, A1AT, AACT, ANXA1, APOA1, CO9, CRP, CSF1, FHL1, FIBG, GELS, HPT, and SAM in the biological sample obtained from the subject differs from a negative control reference level of the least one of A1AG1, A1AT, AACT, ANXA1, APOA1, CO9, CRP, CSF1, FHL1, FIBG, GELS, HPT, and SAM by at least 10%.

219. The method of claim 217, comprising detecting a presence of colorectal cancer in the subject if a level of at least one of A1AG1, A1AT, AACT, ANXA1, APOA1, CO9, CRP, CSF1, FHL1, FIBG, GELS, HPT, and SAM in the biological sample obtained from the subject differs from a positive control reference level of the least one of A1AG1, A1AT, AACT, ANXA1, APOA1, CO9, CRP, CSF1, FHL1, FIBG, GELS, HPT, and SAM by less than 10%.

220. The method of claim 216, wherein the 13 biomarkers are A1AG1, A1AT, AMY2B, CLUS, CO9, ECH1, FRIL, GELS, OSTP, SBP1, SEPR, SPON2, and TIMP1.

221. The method of claim 220, comprising detecting a presence of colorectal cancer in the subject if a level of at least one of A1AG1, A1AT, AMY2B, CLUS, CO9, ECH1, FRIL, GELS, OSTP, SBP1, SEPR, SPON2, and TIMP1 in the biological sample obtained from the subject differs from a negative control reference level of the least one of A1AG1, A1AT, AMY2B, CLUS, CO9, ECH1, FRIL, GELS, OSTP, SBP1, SEPR, SPON2, and TIMP1 by at least 10%.

222. The method of claim 220, comprising detecting a presence of colorectal cancer in the subject if a level of at least one of A1AG1, A1AT, AMY2B, CLUS, CO9, ECH1, FRIL, GELS, OSTP, SBP1, SEPR, SPON2, and TIMP1 in the biological sample obtained from the subject differs from a positive control reference level of the least one of A1AG1, A1AT, AMY2B, CLUS, CO9, ECH1, FRIL, GELS, OSTP, SBP1, SEPR, SPON2, and TIMP1 by less than 10%.

223. The method of claim 192, wherein the biomarker panel comprises at least five biomarkers in the biological sample of the subject.

224. The method of claim 223, wherein the at least five biomarkers comprise AACT, CO3, CO9, CRP, and GELS.

225. The method of claim 224, comprising detecting a presence of colorectal cancer in the subject if a level of at least one of AACT, CO3, CO9, CRP, and GELS in the biological sample obtained from the subject differs from a negative control reference level of the least one of AACT, CO3, CO9, CRP, and GELS by at least 10%.

226. The method of claim 224, comprising detecting a presence of colorectal cancer in the subject if a level of at least one of AACT, CO3, CO9, CRP, and GELS in the biological sample obtained from the subject differs from a positive control reference level of the least one of AACT, CO3, CO9, CRP, and GELS by less than 10%.

227. The method of claim 223, wherein the at least five biomarkers comprise A1AT, CO3, FIBG, GELS, and SPB6.

228. The method of claim 227, comprising detecting a presence of colorectal cancer in the subject if a level of at least one of A1AT, CO3, FIBG, GELS, and SPB6 in the biological sample obtained from the subject differs from a negative control reference level of the least one of A1AT, CO3, FIBG, GELS, and SPB6 by at least 10%.

229. The method of claim 227, comprising detecting a presence of colorectal cancer in the subject if a level of at least one of A1AT, CO3, FIBG, GELS, and SPB6 in the biological sample obtained from the subject differs from a positive control reference level of the least one of A1AT, CO3, FIBG, GELS, and SPB6 by less than 10%.

230. The method of claim 223, wherein the at least five biomarkers comprise CRP, DPP4, SBP1, SEPR, and SRC.

231. The method of claim 230, comprising detecting a presence of colorectal cancer in the subject if a level of at least one of CRP, DPP4, SBP1, SEPR, and SRC in the biological sample obtained from the subject differs from a negative control reference level of the least one of CRP, DPP4, SBP1, SEPR, and SRC by at least 10%.

232. The method of claim 230, comprising detecting a presence of colorectal cancer in the subject if a level of at least one of CRP, DPP4, SBP1, SEPR, and SRC in the biological sample obtained from the subject differs from a positive control reference level of the least one of CRP, DPP4, SBP1, SEPR, and SRC by less than 10%.

233. The method of claim 230, wherein the subject is a male subject.

234. The method of claim 167, wherein the biomarker panel comprises no more than five biomarkers.

235. The method of claim 167, wherein the biomarker panel does not comprise CO3-desARg, ORM, CO3, CO9, GELS, CRP, SAA2, or CEA.

236. A method of treating colorectal cancer in a subject, comprising:
(a) determining a first ratio of a level of a first biomarker which is APOA1 to a level of a second biomarker in a biological sample obtained from the subject;
(b) detecting a presence or absence of colorectal cancer in the subject based upon the determining; and
(c) treating the colorectal cancer in the subject based upon the detecting.

237. A method of treating colorectal cancer in a subject, comprising:
(a) determining a first ratio of a level of a first biomarker which is APOA1 to a level of a second biomarker in a biological sample obtained from the subject;
(b) detecting a presence or absence of colorectal cancer in the subject based upon the determining; and
(c) recommending to the subject at least one of a colonoscopy, sigmoidoscopy, and tissue biopsy to confirm a diagnosis of colorectal cancer in the subject based upon the detecting.

238. The method of claim 236 or 237, wherein the second biomarker is selected from the group consisting of CO3, CO9, A1AT, and FIBG.

239. The method of claim 238, wherein the first ratio is a ratio of APOA1 to CO3.

240. The method of claim 239, comprising detecting a presence of colorectal cancer in the subject if the ratio of APOA1 to CO3 differs from a negative control reference ratio of APOA1 to CO3 by at least 10%.

241. The method of claim 239, comprising detecting a presence of colorectal cancer in the subject if the ratio of APOA1 to CO3 differs from a positive control reference ratio of APOA1 to CO3 by less than 10%.

242. The method of claim 238, wherein the first ratio is a ratio of APOA1 to CO9.

243. The method of claim 242, comprising detecting a presence of colorectal cancer in the subject if the ratio of APOA1 to CO9 differs from a negative control reference ratio of APOA1 to CO9 by at least 10%.

244. The method of claim 242, comprising detecting a presence of colorectal cancer in the subject if the ratio of APOA1 to CO9 differs from a positive control reference ratio of APOA1 to CO9 by less than 10%.

245. The method of claim 238, wherein the first ratio is a ratio of A1AT to APOA1.

246. The method of claim 245, comprising detecting a presence of colorectal cancer in the subject if the ratio of A1AT to APOA1 differs from a negative control reference ratio of A1AT to APOA1 by at least 10%.

247. The method of claim 245, comprising detecting a presence of colorectal cancer in the subject if the ratio of A1AT to APOA1 differs from a positive control reference ratio of A1AT to APOA1 by less than 10%.

248. The method of claim 238, wherein the first ratio is a ratio of APOA1 to FIBG.

249. The method of claim 248, comprising detecting a presence of colorectal cancer in the subject if the ratio of APOA1 to FIBG differs from a negative control reference ratio of APOA1 to FIBG by at least 10%.

250. The method of claim 248, comprising detecting a presence of colorectal cancer in the subject if the ratio of APOA1 to FIBG differs from a positive control reference ratio of APOA1 to FIBG by less than 10%.

251. The method of any of claims 236-250, wherein (b) further comprises determining a second ratio of a level of the first biomarker which is APOA1 to a level of a third biomarker in the biological sample of the subject.

252. The method of claim 251, wherein the third biomarker is selected from the group consisting of CO3, CO9, A1AT, and FIBG.

253. The method of claim 252, wherein the first ratio is a ratio of APOA1 to CO3 and the second ratio is a ratio of APOA1 to CO9.

254. The method of claim 252, wherein the first ratio is a ratio of A1AT to APOA1 and the second ratio is a ratio of APOA1 to FIBG.

255. The method of claim 253, comprising detecting a presence of colorectal cancer in the subject if at least one of: the first ratio differs from a negative control reference first ratio by at least 10%, the second ratio differs from a negative control reference second ratio by at least 10%, the first ratio differs from a positive control reference first ratio by less than 10%, and the second ratio differs from a positive control reference second ratio by less than 10%.

256. A method of treating colorectal cancer in a subject, comprising:
(a) determining a ratio of a level of a first biomarker which is A1AT to a level of a second biomarker which is TRFE in a biological sample obtained from the subject;
(b) detecting a presence or absence of colorectal cancer in the subject based upon the determining; and
(c) treating the colorectal cancer in the subject based upon the detecting.

257. A method of treating colorectal cancer in a subject, comprising:
(a) determining a ratio of a level of a first biomarker which is A1AT to a level of a second biomarker which is TRFE in a biological sample obtained from the subject;
(b) detecting a presence or absence of colorectal cancer in the subject based upon the determining; and
(c) recommending to the subject at least one of a colonoscopy, sigmoidoscopy, and tissue biopsy to confirm a diagnosis of colorectal cancer in the subject based upon the detecting.

258. The method of claim 256 or 257, wherein the subject is male.

259. The method of any of claims 256-258, comprising detecting a presence of colorectal cancer in the subject if the ratio differs from a negative control reference ratio by at least 10%.

260. The method of any of claims 256-258, comprising detecting a presence of colorectal cancer in the subject if the ratio differs from a positive control reference ratio by less than 10%.

261. The method of any one of claims 167-260, wherein the biological sample is selected from the group consisting of whole blood, serum, plasma, blood constituent, bone marrow, saliva, cheek swab, urine, stool, lymph fluid, CNS fluid, and lesion exudate.

262. The method of claim 261, wherein the biological sample is a blood sample.

263. The method of claim 262, wherein the blood sample is a whole blood sample.

264. The method of claim 262, wherein the blood sample is a plasma sample.

265. The method of claim 262, wherein the blood sample is a serum sample.

266. The method of any one of claims 167-265, wherein the subject is a human subject.

267. The method of claim 266, wherein the subject is asymptomatic for colorectal cancer.

268. The method of claim 266, wherein the subject is at least 30 years of age or older.

269. The method of claim 266, wherein the subject is at least 40 years of age or older.

270. The method of claim 266, wherein the subject is at least 50 years of age or older.

271. The method of claim 266, wherein the subject has one or more of: a symptom of colorectal cancer, a family history of colorectal cancer, and a risk factor for colorectal cancer.

272. The method of claim 167, wherein the subject has a previous history of at least one of a colorectal polyp, an adenoma, and CRC.

273. The method of claim 167, wherein the measuring comprises detecting or measuring a level of a fragment, antigen, or transition ion of the at least two biomarkers.

274. The method of claim 167, wherein the measuring comprises use of at least one of: an immunoassay, flow cytometry assay, biochip assay, mass spectrometry assay, and HPLC assay.

275. A computer system for detecting a presence or absence of at least one of an advanced colorectal adenoma and colorectal cancer in a subject, the computer system comprising:
(a) a memory unit for receiving data comprising measurement of a biomarker panel from a biological sample of the subject, wherein the biomarker panel comprises at least two biomarkers selected from the group consisting of A1AG1, A1AT, AACT, AMY2B, ANXA1, APOA1, CAH1, CATD, CEAM3, CLUS, CTNB1, CO3, CO9, CRP, CSF1, DPP4, ECH1, FHL1, FIBB, FIBG, FRIL, GELS, HPT, OSTP, PRDX1, SAA1, SBP1, SEPR, SPB6, SPON2, SYG, TIMP1, and TRFE;
(b) computer-executable instructions for analyzing the measurement data according to a method of any of the preceding claims; and
(c) computer-executable instructions for determining a presence or absence of at least one of advanced colorectal adenoma and colorectal cancer in the subject based upon the analyzing.

276. The computer system of claim 275, further comprising computer-executable instructions to generate a report of the presence or absence of the at least one of an advanced colorectal adenoma and colorectal cancer in the subject.

277. The computer system of claim 276, further comprising a user interface configured to communicate or display said report to a user.

278. A computer readable medium comprising:
(a) computer-executable instructions for analyzing data comprising measurement of a biomarker panel from a biological sample obtained from a subject, wherein the biomarker panel comprises at least two biomarkers selected from the group consisting of A1AG1, A1AT, AACT, AMY2B, ANXA1, APOA1, CAH1, CATD, CEAM3, CLUS, CTNB1, CO3, CO9, CRP, CSF1, DPP4, ECH1, FHL1, FIBB, FIBG, FRIL, GELS, HPT, OSTP, PRDX1, SAM, SBP1, SEPR, SPB6, SPON2, SYG, TIMP1, and TRFE; and
(b) computer-executable instructions for determining a presence or absence of at least one of advanced colorectal adenoma and colorectal cancer in the subject based upon the analyzing.

279. A kit, comprising:
(c) one or more compositions for use in measuring a biomarker panel in a biological sample obtained from a subject, wherein the biomarker panel comprises at least two biomarkers selected from the group consisting of A1AG1, A1AT, AACT, AMY2B, ANXA1, APOA1, CAH1, CATD, CEAM3, CLUS, CTNB1, CO3, CO9, CRP, CSF1, DPP4, ECH1, FHL1, FIBB, FIBG, FRIL, GELS, HPT, OSTP, PRDX1, SAA1, SBP1, SEPR, SPB6, SPON2, SYG, TIMP1, and TRFE; and
(d) instructions for performing a method of any of the preceding claims.

280. The kit of claim 279, further comprising a computer readable medium of claim 278.

281. A method of treating at least one of a colorectal cancer and advanced colorectal adenoma in a subject, comprising:
(a) measuring a biomarker panel in a biological sample obtained from the subject, wherein the biomarker panel comprises at least two biomarkers selected from the group consisting of A1AG1, A1AT, AACT, APOA1, CATD, CEAM3, CLUS, CO3, CO9, CRP, FIBB, FIBG, GELS, OSTP, PRDX1, SAA1, SBP1, and SEPR;
(b) detecting a presence or absence of colorectal cancer and/or advanced colorectal adenoma in the subject based upon the measuring; and
(c) treating the colorectal cancer in the subject based upon the detecting.

282. A method of diagnosing at least one of a colorectal cancer and advanced colorectal adenoma in a subject, comprising:
(a) measuring a biomarker panel in a biological sample obtained from the subject, wherein the biomarker panel comprises at least two biomarkers selected from the group consisting of A1AG1, A1AT, AACT, APOA1, CATD, CEAM3, CLUS, CO3, CO9, CRP, FIBB, FIBG, GELS, OSTP, PRDX1, SAA1, SBP1, and SEPR;
(b) detecting a presence or absence of at least one of colorectal cancer and advanced colorectal adenoma in the subject based upon the measuring; and
(c) recommending to the subject at least one of a colonoscopy, sigmoidoscopy, and tissue biopsy in the subject based upon the detecting.

283. A method, comprising:
(a) obtaining data comprising a measurement of a biomarker panel in a biological sample obtained from a subject, wherein the biomarker panel comprises at least two biomarkers selected from the group consisting of A1AG1, A1AT, AACT, APOA1, CATD, CEAM3, CLUS, CO3, CO9, CRP, FIBB, FIBG, GELS, OSTP, PRDX1, SAA1, SBP1, and SEPR;
(b) generating a subject-specific profile of the biomarker panel based upon the measurement data;
(c) comparing the subject-specific profile of the biomarker panel to a reference profile of the biomarker panel; and
(d) determining a likelihood of at least one of advanced colorectal adenoma and colorectal cancer based upon (c).

284. A method of treating at least one of a colorectal cancer and advanced colorectal adenoma in a subject, comprising:
(a) measuring a biomarker panel in a biological sample obtained from the subject, wherein the biomarker panel comprises at least two biomarkers selected from the group consisting of A1AG1, A1AT, AACT, APOA1, CATD, CEAM3, CLUS, CO3, CO9, CRP, FIBB, FIBG, GELS, OSTP, PRDX1, SAA1, SBP1, and SEPR;
(b) detecting a presence or absence of colorectal cancer and/or advanced colorectal adenoma in the subject based upon the measuring; and
(c) treating the colorectal cancer in the subject based upon the detecting.

285. A method of diagnosing at least one of a colorectal cancer and advanced colorectal adenoma in a subject, comprising:
(a) measuring a biomarker panel in a biological sample obtained from the subject, wherein the biomarker panel comprises at least two biomarkers selected from the group consisting of A1AG1, A1AT, AACT, APOA1, CATD, CEAM3, CLUS, CO3, CO9, CRP, FIBB, FIBG, GELS, OSTP, PRDX1, SAA1, SBP1, and SEPR;
(b) detecting a presence or absence of at least one of colorectal cancer and advanced colorectal adenoma in the subject based upon the measuring; and
(c) recommending to the subject at least one of a colonoscopy, sigmoidoscopy, and tissue biopsy in the subject based upon the detecting.

286. A method, comprising:
(a) obtaining data comprising a measurement of a biomarker panel in a biological sample obtained from a subject, wherein the biomarker panel comprises at least two biomarkers selected from the group consisting of A1AG1, A1AT, AACT, APOA1, CATD, CEAM3, CLUS, CO3, CO9, CRP, FIBB, FIBG, GELS, OSTP, PRDX1, SAA1, SBP1, and SEPR;
(b) generating a subject-specific profile of the biomarker panel based upon the measurement data;
(c) comparing the subject-specific profile of the biomarker panel to a reference profile of the biomarker panel; and
(d) determining a likelihood of at least one of advanced colorectal adenoma and colorectal cancer based upon (c).

287. A computer system for detecting a presence or absence of at least one of an advanced colorectal adenoma and colorectal cancer in a subject, the computer system comprising:
(a) a memory unit for receiving data comprising measurement of a biomarker panel from a biological sample of the subject, wherein the biomarker panel comprises at least two biomarkers selected from the group consisting of A1AG1, A1AT, AACT, APOA1, CATD, CEAM3, CLUS, CO3, CO9, CRP, FIBB, FIBG, GELS, OSTP, PRDX1, SAA1, SBP1, and SEPR;
(b) computer-executable instructions for analyzing the measurement data according to a method of any of the preceding claims; and
(c) computer-executable instructions for determining a presence or absence of at least one of advanced colorectal adenoma and colorectal cancer in the subject based upon the analyzing.

288. A computer system for detecting a presence or absence of at least one of an advanced colorectal adenoma and colorectal cancer in a subject, the computer system comprising:
(a) a memory unit for receiving data comprising measurement of a biomarker panel from a biological sample of the subject, wherein the biomarker panel comprises at least two biomarkers selected from the group consisting of A1AG1, A1AT, AACT, APOA1, CATD, CEAM3, CLUS, CO3, CO9, CRP, FIBB, FIBG, GELS, OSTP, PRDX1, SAA1, SBP1, and SEPR;
(b) computer-executable instructions for analyzing the measurement data according to a method of any of the preceding claims; and
(c) computer-executable instructions for determining a presence or absence of at least one of advanced colorectal adenoma and colorectal cancer in the subject based upon the analyzing.

289. A computer readable medium comprising:
(a) computer-executable instructions for analyzing data comprising measurement of a biomarker panel from a biological sample obtained from a subject, wherein the biomarker panel comprises at least two biomarkers selected from the group consisting of A1AG1, A1AT, AACT, APOA1, CATD, CEAM3, CLUS, CO3, CO9, CRP, FIBB, FIBG, GELS, OSTP, PRDX1, SAA1, SBP1, and SEPR; and
(b) computer-executable instructions for determining a presence or absence of at least one of advanced colorectal adenoma and colorectal cancer in the subject based upon the analyzing.

290. A kit, comprising:
(a) one or more compositions for use in measuring a biomarker panel in a biological sample obtained from a subject, wherein the biomarker panel comprises at least two biomarkers selected from the group consisting of A1AG1, A1AT, AACT, APOA1, CATD, CEAM3, CLUS, CO3, CO9, CRP, FIBB, FIBG, GELS, OSTP, PRDX1, SAA1, SBP1, and SEPR; and
(b) instructions for performing a method of any of the preceding claims.

291. A computer readable medium comprising:
(a) computer-executable instructions for analyzing data comprising measurement of a biomarker panel from a biological sample obtained from a subject, wherein the biomarker panel comprises at least two biomarkers selected from the group consisting of A1AG1, A1AT, AACT, APOA1, CATD, CEAM3, CLUS, CO3, CO9, CRP, FIBB, FIBG, GELS, OSTP, PRDX1, SAA1, SBP1, and SEPR; and
(b) computer-executable instructions for determining a presence or absence of at least one of advanced colorectal adenoma and colorectal cancer in the subject based upon the analyzing.

292. A kit, comprising:
(a) one or more compositions for use in measuring a biomarker panel in a biological sample obtained from a subject, wherein the biomarker panel comprises at least two biomarkers selected from the group consisting of A1AG1, A1AT, AACT, APOA1, CATD, CEAM3, CLUS, CO3, CO9, CRP, FIBB, FIBG, GELS, OSTP, PRDX1, SAA1, SBP1, and SEPR; and
(b) instructions for performing a method of any of the preceding claims.

293. A method of diagnosing at least one of a colorectal cancer and/or advanced colorectal adenoma in a subject, comprising:
obtaining a blood sample from the individual;
determining protein accumulation levels for a panel of proteins in the blood sample;
comparing protein accumulation levels of a panel of proteins in a sample obtained from said individual to levels corresponding to a known cancer status, and
categorizing said colorectal cancer status of said individual as said known colorectal cancer status if said individual's colorectal cancer status panel protein accumulation levels substantially match said colorectal cancer status panel protein accumulation levels corresponding to said known colorectal cancer status, wherein the biomarker panel comprises at least two biomarkers selected from the group consisting of A1AG1, A1AT, AACT, AMY2B, ANXA1, APOA1, CAH1, CATD, CEAM3, CLUS, CO3, CO9, CRP, CSF1, CTNB1, DPP4, ECH1, FHL1, FIBB, FIBG, FRIL, GELS, HPT, OSTP, PRDX1, SAA1, SBP1, SEPR, SPB6, SPON2, SRC, SYG, TIMP, TIMP1, and TRFE.

294. The method of claim 293, wherein the biomarker panel comprises at least three biomarkers selected from the group consisting of A1AG1, A1AT, AACT, AMY2B, ANXA1, APOA1, CAH1, CATD, CEAM3, CLUS, CO3, CO9, CRP, CSF1, CTNB1, DPP4, ECH1, FHL1, FIBB, FIBG, FRIL, GELS, HPT, OSTP, PRDX1, SAA1, SBP1, SEPR, SPB6, SPON2, SRC, SYG, TIMP, TIMP1, and TRFE.

295. The method of claim 293, wherein the biomarker panel comprises at least four biomarkers selected from the group consisting of A1AG1, A1AT, AACT, AMY2B, ANXA1, APOA1, CAH1, CATD, CEAM3, CLUS, CO3, CO9, CRP, CSF1, CTNB1, DPP4, ECH1, FHL1, FIBB, FIBG, FRIL, GELS, HPT, OSTP, PRDX1, SAA1, SBP1, SEPR, SPB6, SPON2, SRC, SYG, TIMP, TIMP1, and TRFE.

296. The method of claim 293, wherein the biomarker panel comprises at least five biomarkers selected from the group consisting of A1AG1, A1AT, AACT, AMY2B, ANXA1, APOA1, CAH1, CATD, CEAM3, CLUS, CO3, CO9, CRP, CSF1, CTNB1, DPP4, ECH1, FHL1, FIBB, FIBG, FRIL, GELS, HPT, OSTP, PRDX1, SAA1, SBP1, SEPR, SPB6, SPON2, SRC, SYG, TIMP, TIMP1, and TRFE.

297. The method of claim 293, wherein the biomarker panel comprises at least six biomarkers selected from the group consisting of A1AG1, A1AT, AACT, AMY2B, ANXA1, APOA1, CAH1, CATD, CEAM3, CLUS, CO3, CO9, CRP, CSF1, CTNB1, DPP4, ECH1, FHL1, FIBB, FIBG, FRIL, GELS, HPT, OSTP, PRDX1, SAA1, SBP1, SEPR, SPB6, SPON2, SRC, SYG, TIMP, TIMP1, and TRFE.

298. The method of claim 293, wherein the biomarker panel comprises at least seven biomarkers selected from the group consisting of A1AG1, A1AT, AACT, AMY2B, ANXA1, APOA1, CAH1, CATD, CEAM3, CLUS, CO3, CO9, CRP, CSF1, CTNB1, DPP4, ECH1, FHL1, FIBB, FIBG, FRIL, GELS, HPT, OSTP, PRDX1, SAA1, SBP1, SEPR, SPB6, SPON2, SRC, SYG, TIMP, TIMP1, and TRFE.

299. The method of claim 293, wherein the biomarker panel comprises at least eight biomarkers selected from the group consisting of A1AG1, A1AT, AACT, AMY2B, ANXA1, APOA1, CAH1, CATD, CEAM3, CLUS, CO3, CO9, CRP, CSF1, CTNB1, DPP4, ECH1, FHL1, FIBB, FIBG, FRIL, GELS, HPT, OSTP, PRDX1, SAA1, SBP1, SEPR, SPB6, SPON2, SRC, SYG, TIMP, TIMP1, and TRFE.

300. The method of claim 293, wherein the biomarker panel comprises at least nine biomarkers selected from the group consisting of A1AG1, A1AT, AACT, AMY2B, ANXA1, APOA1, CAH1, CATD, CEAM3, CLUS, CO3, CO9, CRP, CSF1, CTNB1, DPP4, ECH1, FHL1, FIBB, FIBG, FRIL, GELS, HPT, OSTP, PRDX1, SAA1, SBP1, SEPR, SPB6, SPON2, SRC, SYG, TIMP, TIMP1, and TRFE.

301. The method of claim 293, wherein the biomarker panel comprises at least ten biomarkers selected from the group consisting of A1AG1, A1AT, AACT, AMY2B, ANXA1, APOA1, CAH1, CATD, CEAM3, CLUS, CO3, CO9, CRP, CSF1, CTNB1, DPP4, ECH1, FHL1, FIBB, FIBG, FRIL, GELS, HPT, OSTP, PRDX1, SAA1, SBP1, SEPR, SPB6, SPON2, SRC, SYG, TIMP, TIMP1, and TRFE.

302. The method of claim 293, wherein the biomarker panel comprises at least eleven biomarkers selected from the group consisting of A1AG1, A1AT, AACT, AMY2B, ANXA1, APOA1, CAH1, CATD, CEAM3, CLUS, CO3, CO9, CRP, CSF1, CTNB1, DPP4, ECH1, FHL1, FIBB, FIBG, FRIL, GELS, HPT, OSTP, PRDX1, SAA1, SBP1, SEPR, SPB6, SPON2, SRC, SYG, TIMP, TIMP1, and TRFE.

303. The method of claim 293, wherein the biomarker panel comprises at least twelve biomarkers selected from the group consisting of A1AG1, A1AT, AACT, AMY2B, ANXA1, APOA1, CAH1, CATD, CEAM3, CLUS, CO3, CO9, CRP, CSF1, CTNB1, DPP4, ECH1, FHL1, FIBB, FIBG, FRIL, GELS, HPT, OSTP, PRDX1, SAA1, SBP1, SEPR, SPB6, SPON2, SRC, SYG, TIMP, TIMP1, and TRFE.

304. The method of claim 293, wherein the biomarker panel comprises at least thirteen biomarkers selected from the group consisting of A1AG1, A1AT, AACT, AMY2B, ANXA1, APOA1, CAH1, CATD, CEAM3, CLUS, CO3, CO9, CRP, CSF1, CTNB1, DPP4, ECH1, FHL1, FIBB, FIBG, FRIL, GELS, HPT, OSTP, PRDX1, SAA1, SBP1, SEPR, SPB6, SPON2, SRC, SYG, TIMP, TIMP1, and TRFE.

305. The method of claim 293, wherein the biomarker panel comprises CO9 and GELS, and wherein the categorizing has an area under the receiver operating characteristic curve greater than 0.76.

306. The method of claim 293, wherein the biomarker panel comprises A1AG1, A1AT, AACT, ANXA1, APOA1, CO9, CRP, CSF1, FHL1, FIBG, GELS, HPT, and SAM.

307. The method of claim 293, wherein the biomarker panel comprises A1AG1, A1AT, AACT, ANXA1, APOA1, CO9, CRP, CSF1, FHL1, FIBG, GELS, HPT, and SAM, and wherein the categorizing has an area under the receiver operating characteristic curve greater than 0.83.

308. The method of claim 293, wherein the biomarker panel comprises APOA1, CO3, and CO9.

309. The method of claim 293, wherein the biomarker panel comprises APOA1, CO3, and CO9, and wherein the categorizing has an area under the receiver operating characteristic curve greater than 0.81.

310. The method of claim 293, wherein the biomarker panel comprises AACT, CO3, CO9, CRP, and GELS.

311. The method of claim 293, wherein the biomarker panel comprises AACT, CO3, CO9, CRP, and GELS, and wherein the categorizing has an area under the receiver operating characteristic curve greater than 0.81.

312. The method of claim 293, wherein the biomarker panel comprises SPB6, GELS, A1AT, FIBG, and CO3.

313. The method of claim 293, wherein the biomarker panel comprises SPB6, GELS, A1AT, FIBG, and CO3, and wherein the categorizing has an area under the receiver operating characteristic curve greater than 0.79.

314. The method of claim 293, wherein the biomarker panel comprises A1AG1, A1AT, AMY2B, CLUS, CO9, ECH1, FRIL, GELS, OSTP, SBP1, SEPR, SPON2, and TIMP1.

315. The method of claim 293, wherein the biomarker panel comprises A1AG1, A1AT, AMY2B, CLUS, CO9, ECH1, FRIL, GELS, OSTP, SBP1, SEPR, SPON2, and TIMP1, and wherein the categorizing has an area under the receiver operating characteristic curve greater than 0.91.

316. The method of claim 293, wherein the biomarker panel comprises CO9 and GELS, and wherein the categorizing has an area under the receiver operating characteristic curve greater than 0.87.

317. The method of claim 293, wherein the biomarker panel comprises CO9 and GELS.

318. The method of claim 293, wherein the biomarker panel comprises GELS, PRDX1, CO9, and CATD.

319. The method of claim 293, wherein the biomarker panel comprises GELS, PRDX1, CO9, and CATD, and wherein the categorizing has an area under the receiver operating characteristic curve greater than 0.84.

320. The method of claim 293, wherein the biomarker panel comprises A1AT, APOA1, and FIBG.

321. The method of claim 293, wherein the biomarker panel comprises A1AT, APOA1, and FIBG, and wherein the categorizing has an area under the receiver operating characteristic curve greater than 0.81.

322. The method of claim 293, wherein the biomarker panel comprises A1AT and TRFE.

323. The method of claim 293, wherein the biomarker panel comprises A1AT and TRFE, and wherein the categorizing has an area under the receiver operating characteristic curve greater than 0.89.

324. The method of claim 293, wherein the biomarker panel comprises A1AT, APOA1, FIBB, and CEAM3.

325. The method of claim 293, wherein the biomarker panel comprises A1AT, APOA1, FIBB, and CEAM3, and wherein the categorizing has an area under the receiver operating characteristic curve greater than 0.80.

326. The method of claim 293, wherein the biomarker panel comprises CAH1, CRP, FIBG, and CTNB1.

327. The method of claim 293, wherein the biomarker panel comprises CAH1, CRP, FIBG, and CTNB1, and wherein the categorizing has an area under the receiver operating characteristic curve greater than 0.78.

328. The method of claim 293, wherein the biomarker panel comprises CRP, SEPR, SBP1, SRC, and DPP4.

329. The method of claim 293, wherein the biomarker panel comprises CRP, SEPR, SBP1, SRC, and DPP4, and wherein the categorizing has an area under the receiver operating characteristic curve greater than 0.78.

330. The method of claim 293, wherein the biomarker panel comprises CRP and TMP.

331. The method of claim 293, wherein the biomarker panel comprises CRP and TMP, and wherein the categorizing has an area under the receiver operating characteristic curve greater than 0.76.

332. The method of claim 293, wherein the biomarker panel comprises SYG, AACT, and CO9.

333. The method of claim 293, wherein the biomarker panel comprises SYG, AACT, and CO9, and wherein the categorizing has an area under the receiver operating characteristic curve greater than 0.86.

334. The method of any one of claims 180-221, wherein the categorizing has a sensitivity of at least 80%.

335. The method of any one of claims 180-221, wherein the categorizing has a sensitivity of at least 85%.

336. The method of any one of claims 180-221, wherein the categorizing has a sensitivity of at least 90%.

337. The method of any one of claims 180-221, wherein the categorizing has a specificity of at least 80%.

338. The method of any one of claims 180-221, wherein the categorizing has a specificity of at least 85%.

339. The method of any one of claims 180-221, wherein the categorizing has a specificity of at least 90%.
